SEQUENCE LISTING (185 sequences)

SEQ ID NO: 1 - DNA, 28 bp, synthetic construct
  source 1..28: mol_type = other DNA
cagggaaggagtgcgtggctgcctttgt

SEQ ID NO: 2 - DNA, 28 bp, synthetic construct
  source 1..28: mol_type = other DNA
tgtttccgtcggtgcgtgaggaagggac

SEQ ID NO: 3 - DNA, 28 bp, synthetic construct
  source 1..28: mol_type = other DNA
gtcccttcctcacgcaccgacggaaaca

SEQ ID NO: 4 - DNA, 28 bp, synthetic construct
  source 1..28: mol_type = other DNA
gtggttggtgagggtcatctcgctggag

SEQ ID NO: 5 - DNA, 28 bp, synthetic construct
  source 1..28: mol_type = other DNA
acaaaggcagccacgcactccttccctg

SEQ ID NO: 6 - DNA, 28 bp, synthetic construct
  source 1..28: mol_type = other DNA
gaggtcgctctactgggagtggttggtg

SEQ ID NO: 7 - DNA, 28 bp, synthetic construct
  source 1..28: mol_type = other DNA
ctccagcgagatgaccctcaccaaccac

SEQ ID NO: 8 - DNA, 28 bp, synthetic construct
  source 1..28: mol_type = other DNA
caccaaccactcccagtagagcgacctc

SEQ ID NO: 9 - DNA, 41 bp, synthetic construct
  source 1..41: mol_type = other DNA
acaaaggcagccacgcactccttccctgaaggccggaatct

SEQ ID NO: 10 - DNA, 41 bp, synthetic construct
  source 1..41: mol_type = other DNA
gctgccgccactagccatcttactgctgaggactcttcgct

SEQ ID NO: 11 - DNA, 40 bp, synthetic construct
  source 1..40: mol_type = other DNA
gattccggccttgtggttggtgagggtcatctcgctggag

SEQ ID NO: 12 - DNA, 40 bp, synthetic construct
  source 1..40: mol_type = other DNA
gcgaagagtcctggagtgccgccaatgtatgcgagggtga

SEQ ID NO: 13 - DNA, 71 bp, synthetic construct
  source 1..71: mol_type = other DNA
gcgcgcgttttttttgcttgcgtctcctgccagccatatccggtctacgtgatccttttttttcgcgcgct

SEQ ID NO: 14 - DNA, 83 bp, synthetic construct
  source 1..83: mol_type = other DNA
gcgcgcgttttttttttttttgcttgcgtctcctgccagccatatccggtctacgtgatccttttttttttttttcgcgcgct

SEQ ID NO: 15 - DNA, 62 bp, synthetic construct
  source 1..62: mol_type = other DNA
ggatcacgtagattttgcttgcgtctcctgccagccatatccggtttttctacgtgattcct

SEQ ID NO: 16 - DNA, 81 bp, synthetic construct
  source 1..81: mol_type = other DNA
gcgaagagtcctggagtgccgccaatgtatgcgagggtgagctgccgccactagccatcttactgctgaggactcttcgct

SEQ ID NO: 17 - DNA, 93 bp, synthetic construct
  source 1..93: mol_type = other DNA
gcgaagagtcctttttttggagtgccgccaatgtatgcgagggtgagctgccgccactagccatcttactgctgttttttaggactcttcgct

SEQ ID NO: 18 - DNA, 100 bp, synthetic construct
  source 1..100: mol_type = other DNA
gcgaagagtcctttttttggagtgccgccaatgtatgcgagggtgatttttttgctgccgccactagccatcttactgctgttttttaggactcttcgct

SEQ ID NO: 19 - DNA, 81 bp, synthetic construct
  source 1..81: mol_type = other DNA
gattccggccttgtggttggtgagggtcatctcgctggagacaaaggcagccacgcactccttccctgaaggccggaatct

SEQ ID NO: 20 - DNA, 93 bp, synthetic construct
  source 1..93: mol_type = other DNA
gattccggccttttttttgtggttggtgagggtcatctcgctggagacaaaggcagccacgcactccttccctgttttttaaggccggaatct

SEQ ID NO: 21 - DNA, 100 bp, synthetic construct
  source 1..100: mol_type = other DNA
gattccggccttttttttgtggttggtgagggtcatctcgctggagtttttttacaaaggcagccacgcactccttccctgttttttaaggccggaatct

SEQ ID NO: 22 - DNA, 61 bp, synthetic construct
  source 1..61: mol_type = other DNA
ggatcacgtagattttgcttgcgtctcctgccagccatatccggtttttctacgtgatcct

SEQ ID NO: 23 - DNA, 77 bp, synthetic construct
  source 1..77: mol_type = other DNA
ggatcacgtagattttttttttttgcttgcgtctcctgccagccatatccggtttttttttttttctacgtgatcct

SEQ ID NO: 24 - DNA, 101 bp, synthetic construct
  source 1..101: mol_type = other DNA
ggatcacgtagattttttttttttttttttttttttgcttgcgtctcctgccagccatatccggtttttttttttttttttttttttttctacgtgatcct

SEQ ID NO: 25 - DNA, 103 bp, synthetic construct
  source 1..103: mol_type = other DNA
ggatcacgtagattttttttttttttttttttttttttttttttttttttttgcttgcgtctcctgccagccatatccggtttttttttttctacgtgatcct

SEQ ID NO: 26 - DNA, 67 bp, synthetic construct
  source 1..67: mol_type = other DNA
ggatcacgtagattttagatctgcttgcgtctcctgccagccatatccggtttttctacgtgatcct

SEQ ID NO: 27 - DNA, 82 bp, synthetic construct
  source 1..82: mol_type = other DNA
ggatcacgtagattttttttttttagatctgcttgcgtctcctgccagccatatccggttttttttttttctacgtgatcct

SEQ ID NO: 28 - DNA, 41 bp, synthetic construct
  source 1..41: mol_type = other DNA
tgtttccgtcggtgcgtgaggaagggacttccggccttaga

SEQ ID NO: 29 - DNA, 41 bp, synthetic construct
  source 1..41: mol_type = other DNA
cgacggcggtgatcggtagaatgacgactcctgagaagcga

SEQ ID NO: 30 - DNA, 40 bp, synthetic construct
  source 1..40: mol_type = other DNA
ctaaggccggaacaccaaccactcccagtagagcgacctc

SEQ ID NO: 31 - DNA, 40 bp, synthetic construct
  source 1..40: mol_type = other DNA
cgcttctcaggacctcacggcggttacatacgctcccact

SEQ ID NO: 32 - DNA, 71 bp, synthetic construct
  source 1..71: mol_type = other DNA
cgcgcgcaaaaaaaacgaacgcagaggacggtcggtataggccagatgcactaggaaaaaaaagcgcgcga

SEQ ID NO: 33 - DNA, 83 bp, synthetic construct
  source 1..83: mol_type = other DNA
cgcgcgcaaaaaaaaaaaaaacgaacgcagaggacggtcggtataggccagatgcactaggaaaaaaaaaaaaaagcgcgcga

SEQ ID NO: 34 - DNA, 62 bp, synthetic construct
  source 1..62: mol_type = other DNA
cctagtgcatctaaaacgaacgcagaggacggtcggtataggccaaaaagatgcactaagga

SEQ ID NO: 35 - DNA, 81 bp, synthetic construct
  source 1..81: mol_type = other DNA
cgcttctcaggacctcacggcggttacatacgctcccactcgacggcggtgatcggtagaatgacgactcctgagaagcga

SEQ ID NO: 36 - DNA, 93 bp, synthetic construct
  source 1..93: mol_type = other DNA
cgcttctcaggaaaaaaacctcacggcggttacatacgctcccactcgacggcggtgatcggtagaatgacgacaaaaaatcctgagaagcga

SEQ ID NO: 37 - DNA, 100 bp, synthetic construct
  source 1..100: mol_type = other DNA
cgcttctcaggaaaaaaacctcacggcggttacatacgctcccactaaaaaaacgacggcggtgatcggtagaatgacgacaaaaaatcctgagaagcga

SEQ ID NO: 38 - DNA, 81 bp, synthetic construct
  source 1..81: mol_type = other DNA
ctaaggccggaacaccaaccactcccagtagagcgacctctgtttccgtcggtgcgtgaggaagggacttccggccttaga

SEQ ID NO: 39 - DNA, 93 bp, synthetic construct
  source 1..93: mol_type = other DNA
ctaaggccggaaaaaaaacaccaaccactcccagtagagcgacctctgtttccgtcggtgcgtgaggaagggacaaaaaattccggccttaga

SEQ ID NO: 40 - DNA, 100 bp, synthetic construct
  source 1..100: mol_type = other DNA
ctaaggccggaaaaaaaacaccaaccactcccagtagagcgacctcaaaaaaatgtttccgtcggtgcgtgaggaagggacaaaaaattccggccttaga

SEQ ID NO: 41 - DNA, 61 bp, synthetic construct
  source 1..61: mol_type = other DNA
cctagtgcatctaaaacgaacgcagaggacggtcggtataggccaaaaagatgcactagga

SEQ ID NO: 42 - DNA, 77 bp, synthetic construct
  source 1..77: mol_type = other DNA
cctagtgcatctaaaaaaaaaaaacgaacgcagaggacggtcggtataggccaaaaaaaaaaaaagatgcactagga

SEQ ID NO: 43 - DNA, 101 bp, synthetic construct
  source 1..101: mol_type = other DNA
cctagtgcatctaaaaaaaaaaaaaaaaaaaaaaaacgaacgcagaggacggtcggtataggccaaaaaaaaaaaaaaaaaaaaaaaaagatgcactagga

SEQ ID NO: 44 - DNA, 103 bp, synthetic construct
  source 1..103: mol_type = other DNA
cctagtgcatctaaaaaaaaaaaaaaaaaaaaaaaaaaaaaaaaaaaaaaaacgaacgcagaggacggtcggtataggccaaaaaaaaaaagatgcactagga

SEQ ID NO: 45 - DNA, 67 bp, synthetic construct
  source 1..67: mol_type = other DNA
cctagtgcatctaaaatctagacgaacgcagaggacggtcggtataggccaaaaagatgcactagga

SEQ ID NO: 46 - DNA, 82 bp, synthetic construct
  source 1..82: mol_type = other DNA
cctagtgcatctaaaaaaaaaaaatctagacgaacgcagaggacggtcggtataggccaaaaaaaaaaaagatgcactagga

SEQ ID NO: 47 - DNA, 41 bp, synthetic construct
  source 1..41: mol_type = other DNA
agattccggccttcagggaaggagtgcgtggctgcctttgt

SEQ ID NO: 48 - DNA, 41 bp, synthetic construct
  source 1..41: mol_type = other DNA
agcgaagagtcctcagcagtaagatggctagtggcggcagc

SEQ ID NO: 49 - DNA, 40 bp, synthetic construct
  source 1..40: mol_type = other DNA
tcaccctcgcatacattggcggcactccaggactcttcgc

SEQ ID NO: 50 - DNA, 40 bp, synthetic construct
  source 1..40: mol_type = other DNA
ctccagcgagatgaccctcaccaaccacaaggccggaatc

SEQ ID NO: 51 - DNA, 71 bp, synthetic construct
  source 1..71: mol_type = other DNA
agcgcgcgaaaaaaaaggatcacgtagaccggatatggctggcaggagacgcaagcaaaaaaaacgcgcgc

SEQ ID NO: 52 - DNA, 62 bp, synthetic construct
  source 1..62: mol_type = other DNA
aggaatcacgtagaaaaaccggatatggctggcaggagacgcaagcaaaatctacgtgatcc

SEQ ID NO: 53 - DNA, 83 bp, synthetic construct
  source 1..83: mol_type = other DNA
agcgcgcgaaaaaaaaaaaaaaggatcacgtagaccggatatggctggcaggagacgcaagcaaaaaaaaaaaaaacgcgcgc

SEQ ID NO: 54 - DNA, 81 bp, synthetic construct
  source 1..81: mol_type = other DNA
agcgaagagtcctcagcagtaagatggctagtggcggcagctcaccctcgcatacattggcggcactccaggactcttcgc

SEQ ID NO: 55 - DNA, 93 bp, synthetic construct
  source 1..93: mol_type = other DNA
agcgaagagtcctaaaaaacagcagtaagatggctagtggcggcagctcaccctcgcatacattggcggcactccaaaaaaaggactcttcgc

SEQ ID NO: 56 - DNA, 100 bp, synthetic construct
  source 1..100: mol_type = other DNA
agcgaagagtcctaaaaaacagcagtaagatggctagtggcggcagcaaaaaaatcaccctcgcatacattggcggcactccaaaaaaaggactcttcgc

SEQ ID NO: 57 - DNA, 81 bp, synthetic construct
  source 1..81: mol_type = other DNA
agattccggccttcagggaaggagtgcgtggctgcctttgtctccagcgagatgaccctcaccaaccacaaggccggaatc

SEQ ID NO: 58 - DNA, 93 bp, synthetic construct
  source 1..93: mol_type = other DNA
agattccggccttaaaaaacagggaaggagtgcgtggctgcctttgtctccagcgagatgaccctcaccaaccacaaaaaaaaggccggaatc

SEQ ID NO: 59 - DNA, 100 bp, synthetic construct
  source 1..100: mol_type = other DNA
agattccggccttaaaaaacagggaaggagtgcgtggctgcctttgtaaaaaaactccagcgagatgaccctcaccaaccacaaaaaaaaggccggaatc

SEQ ID NO: 60 - DNA, 61 bp, synthetic construct
  source 1..61: mol_type = other DNA
aggatcacgtagaaaaaccggatatggctggcaggagacgcaagcaaaatctacgtgatcc

SEQ ID NO: 61 - DNA, 77 bp, synthetic construct
  source 1..77: mol_type = other DNA
aggatcacgtagaaaaaaaaaaaaaccggatatggctggcaggagacgcaagcaaaaaaaaaaaatctacgtgatcc

SEQ ID NO: 62 - DNA, 101 bp, synthetic construct
  source 1..101: mol_type = other DNA
aggatcacgtagaaaaaaaaaaaaaaaaaaaaaaaaaccggatatggctggcaggagacgcaagcaaaaaaaaaaaaaaaaaaaaaaaatctacgtgatcc

SEQ ID NO: 63 - DNA, 82 bp, synthetic construct
  source 1..82: mol_type = other DNA
aggatcacgtagaaaaaaaaaaaaccggatatggctggcaggagacgcaagcagatctaaaaaaaaaaaatctacgtgatcc

SEQ ID NO: 64 - DNA, 67 bp, synthetic construct
  source 1..67: mol_type = other DNA
aggatcacgtagaaaaaccggatatggctggcaggagacgcaagcagatctaaaatctacgtgatcc

SEQ ID NO: 65 - DNA, 103 bp, synthetic construct
  source 1..103: mol_type = other DNA
aggatcacgtagaaaaaaaaaaaccggatatggctggcaggagacgcaagcaaaaaaaaaaaaaaaaaaaaaaaaaaaaaaaaaaaaaaaatctacgtgatcc

SEQ ID NO: 66 - DNA, 40 bp, synthetic construct
  source 1..40: mol_type = other DNA
agtgggagcgtatgtaaccgccgtgaggtcctgagaagcg

SEQ ID NO: 67 - DNA, 40 bp, synthetic construct
  source 1..40: mol_type = other DNA
gaggtcgctctactgggagtggttggtgttccggccttag

SEQ ID NO: 68 - DNA, 41 bp, synthetic construct
  source 1..41: mol_type = other DNA
tcgcttctcaggagtcgtcattctaccgatcaccgccgtcg

SEQ ID NO: 69 - DNA, 41 bp, synthetic construct
  source 1..41: mol_type = other DNA
tctaaggccggaagtcccttcctcacgcaccgacggaaaca

SEQ ID NO: 70 - DNA, 62 bp, synthetic construct
  source 1..62: mol_type = other DNA
tccttagtgcatctttttggcctataccgaccgtcctctgcgttcgttttagatgcactagg

SEQ ID NO: 71 - DNA, 83 bp, synthetic construct
  source 1..83: mol_type = other DNA
tcgcgcgcttttttttttttttcctagtgcatctggcctataccgaccgtcctctgcgttcgttttttttttttttgcgcgcg

SEQ ID NO: 72 - DNA, 71 bp, synthetic construct
  source 1..71: mol_type = other DNA
tcgcgcgcttttttttcctagtgcatctggcctataccgaccgtcctctgcgttcgttttttttgcgcgcg

SEQ ID NO: 73 - DNA, 81 bp, synthetic construct
  source 1..81: mol_type = other DNA
tcgcttctcaggagtcgtcattctaccgatcaccgccgtcgagtgggagcgtatgtaaccgccgtgaggtcctgagaagcg

SEQ ID NO: 74 - DNA, 100 bp, synthetic construct
  source 1..100: mol_type = other DNA
tcgcttctcaggattttttgtcgtcattctaccgatcaccgccgtcgtttttttagtgggagcgtatgtaaccgccgtgaggtttttttcctgagaagcg

SEQ ID NO: 75 - DNA, 93 bp, synthetic construct
  source 1..93: mol_type = other DNA
tcgcttctcaggattttttgtcgtcattctaccgatcaccgccgtcgagtgggagcgtatgtaaccgccgtgaggtttttttcctgagaagcg

SEQ ID NO: 76 - DNA, 100 bp, synthetic construct
  source 1..100: mol_type = other DNA
tctaaggccggaattttttgtcccttcctcacgcaccgacggaaacatttttttgaggtcgctctactgggagtggttggtgttttttttccggccttag

SEQ ID NO: 77 - DNA, 93 bp, synthetic construct
  source 1..93: mol_type = other DNA
tctaaggccggaattttttgtcccttcctcacgcaccgacggaaacagaggtcgctctactgggagtggttggtgttttttttccggccttag

SEQ ID NO: 78 - DNA, 81 bp, synthetic construct
  source 1..81: mol_type = other DNA
tctaaggccggaagtcccttcctcacgcaccgacggaaacagaggtcgctctactgggagtggttggtgttccggccttag

SEQ ID NO: 79 - DNA, 101 bp, synthetic construct
  source 1..101: mol_type = other DNA
tcctagtgcatctttttttttttttttttttttttttggcctataccgaccgtcctctgcgttcgttttttttttttttttttttttttagatgcactagg

SEQ ID NO: 80 - DNA, 77 bp, synthetic construct
  source 1..77: mol_type = other DNA
tcctagtgcatctttttttttttttggcctataccgaccgtcctctgcgttcgttttttttttttagatgcactagg

SEQ ID NO: 81 - DNA, 61 bp, synthetic construct
  source 1..61: mol_type = other DNA
tcctagtgcatctttttggcctataccgaccgtcctctgcgttcgttttagatgcactagg

SEQ ID NO: 82 - DNA, 82 bp, synthetic construct
  source 1..82: mol_type = other DNA
tcctagtgcatcttttttttttttggcctataccgaccgtcctctgcgttcgtctagattttttttttttagatgcactagg

SEQ ID NO: 83 - DNA, 67 bp, synthetic construct
  source 1..67: mol_type = other DNA
tcctagtgcatctttttggcctataccgaccgtcctctgcgttcgtctagattttagatgcactagg

SEQ ID NO: 84 - DNA, 103 bp, synthetic construct
  source 1..103: mol_type = other DNA
tcctagtgcatctttttttttttggcctataccgaccgtcctctgcgttcgttttttttttttttttttttttttttttttttttttttttagatgcactagg

SEQ ID NO: 85 - DNA, 25 bp, synthetic construct
  source 1..25: mol_type = other DNA
acaaaggcagccacgcactccttcc

SEQ ID NO: 86 - DNA, 16 bp, synthetic construct
  source 1..16: mol_type = other DNA
ctccagcgagatgacc

SEQ ID NO: 87 - DNA, 25 bp, synthetic construct
  source 1..25: mol_type = other DNA
ctccagcgagatgaccctcaccaac

SEQ ID NO: 88 - DNA, 16 bp, synthetic construct
  source 1..16: mol_type = other DNA
acaaaggcagccacgc

SEQ ID NO: 89 - DNA, 22 bp, synthetic construct
  source 1..22: mol_type = other DNA
ctccagcgagatgaccctcacc

SEQ ID NO: 90 - DNA, 19 bp, synthetic construct
  source 1..19: mol_type = other DNA
acaaaggcagccacgcact

SEQ ID NO: 91 - DNA, 19 bp, synthetic construct
  source 1..19: mol_type = other DNA
ctccagcgagatgaccctc

SEQ ID NO: 92 - DNA, 22 bp, synthetic construct
  source 1..22: mol_type = other DNA
acaaaggcagccacgcactcct

SEQ ID NO: 93 - DNA, 25 bp, synthetic construct
  source 1..25: mol_type = other DNA
ccttcctcacgcaccgacggaaaca

SEQ ID NO: 94 - DNA, 25 bp, synthetic construct
  source 1..25: mol_type = other DNA
caaccactcccagtagagcgacctc

SEQ ID NO: 95 - DNA, 22 bp, synthetic construct
  source 1..22: mol_type = other DNA
tcctcacgcaccgacggaaaca

SEQ ID NO: 96 - DNA, 22 bp, synthetic construct
  source 1..22: mol_type = other DNA
ccactcccagtagagcgacctc

SEQ ID NO: 97 - DNA, 19 bp, synthetic construct
  source 1..19: mol_type = other DNA
tcacgcaccgacggaaaca

SEQ ID NO: 98 - DNA, 19 bp, synthetic construct
  source 1..19: mol_type = other DNA
ctcccagtagagcgacctc

SEQ ID NO: 99 - DNA, 16 bp, synthetic construct
  source 1..16: mol_type = other DNA
cgcaccgacggaaaca

SEQ ID NO: 100 - DNA, 16 bp, synthetic construct
  source 1..16: mol_type = other DNA
ccagtagagcgacctc

SEQ ID NO: 101 - DNA, 25 bp, synthetic construct
  source 1..25: mol_type = other DNA
ggaaggagtgcgtggctgcctttgt

SEQ ID NO: 102 - DNA, 25 bp, synthetic construct
  source 1..25: mol_type = other DNA
gttggtgagggtcatctcgctggag

SEQ ID NO: 103 - DNA, 22 bp, synthetic construct
  source 1..22: mol_type = other DNA
aggagtgcgtggctgcctttgt

SEQ ID NO: 104 - DNA, 22 bp, synthetic construct
  source 1..22: mol_type = other DNA
ggtgagggtcatctcgctggag

SEQ ID NO: 105 - DNA, 19 bp, synthetic construct
  source 1..19: mol_type = other DNA
agtgcgtggctgcctttgt

SEQ ID NO: 106 - DNA, 19 bp, synthetic construct
  source 1..19: mol_type = other DNA
gagggtcatctcgctggag

SEQ ID NO: 107 - DNA, 16 bp, synthetic construct
  source 1..16: mol_type = other DNA
gcgtggctgcctttgt

SEQ ID NO: 108 - DNA, 16 bp, synthetic construct
  source 1..16: mol_type = other DNA
ggtcatctcgctggag

SEQ ID NO: 109 - DNA, 25 bp, synthetic construct
  source 1..25: mol_type = other DNA
tgtttccgtcggtgcgtgaggaagg

SEQ ID NO: 110 - DNA, 25 bp, synthetic construct
  source 1..25: mol_type = other DNA
gaggtcgctctactgggagtggttg

SEQ ID NO: 111 - DNA, 22 bp, synthetic construct
  source 1..22: mol_type = other DNA
tgtttccgtcggtgcgtgagga

SEQ ID NO: 112 - DNA, 22 bp, synthetic construct
  source 1..22: mol_type = other DNA
gaggtcgctctactgggagtgg

SEQ ID NO: 113 - DNA, 19 bp, synthetic construct
  source 1..19: mol_type = other DNA
tgtttccgtcggtgcgtga

SEQ ID NO: 114 - DNA, 19 bp, synthetic construct
  source 1..19: mol_type = other DNA
gaggtcgctctactgggag

SEQ ID NO: 115 - DNA, 16 bp, synthetic construct
  source 1..16: mol_type = other DNA
tgtttccgtcggtgcg

SEQ ID NO: 116 - DNA, 16 bp, synthetic construct
  source 1..16: mol_type = other DNA
gaggtcgctctactgg

SEQ ID NO: 117 - DNA, 25 bp, synthetic construct
  source 1..25: mol_type = other DNA
aaggcagccacgcactccttccctg

SEQ ID NO: 118 - DNA, 25 bp, synthetic construct
  source 1..25: mol_type = other DNA
cagcgagatgaccctcaccaaccac

SEQ ID NO: 119 - DNA, 22 bp, synthetic construct
  source 1..22: mol_type = other DNA
gcagccacgcactccttccctg

SEQ ID NO: 120 - DNA, 22 bp, synthetic construct
  source 1..22: mol_type = other DNA
cgagatgaccctcaccaaccac

SEQ ID NO: 121 - DNA, 19 bp, synthetic construct
  source 1..19: mol_type = other DNA
gccacgcactccttccctg

SEQ ID NO: 122 - DNA, 19 bp, synthetic construct
  source 1..19: mol_type = other DNA
gatgaccctcaccaaccac

SEQ ID NO: 123 - DNA, 16 bp, synthetic construct
  source 1..16: mol_type = other DNA
acgcactccttccctg

SEQ ID NO: 124 - DNA, 16 bp, synthetic construct
  source 1..16: mol_type = other DNA
gaccctcaccaaccac

SEQ ID NO: 125 - DNA, 25 bp, synthetic construct
  source 1..25: mol_type = other DNA
gtcccttcctcacgcaccgacggaa

SEQ ID NO: 126 - DNA, 25 bp, synthetic construct
  source 1..25: mol_type = other DNA
caccaaccactcccagtagagcgac

SEQ ID NO: 127 - DNA, 22 bp, synthetic construct
  source 1..22: mol_type = other DNA
gtcccttcctcacgcaccgacg

SEQ ID NO: 128 - DNA, 22 bp, synthetic construct
  source 1..22: mol_type = other DNA
caccaaccactcccagtagagc

SEQ ID NO: 129 - DNA, 19 bp, synthetic construct
  source 1..19: mol_type = other DNA
gtcccttcctcacgcaccg

SEQ ID NO: 130 - DNA, 19 bp, synthetic construct
  source 1..19: mol_type = other DNA
caccaaccactcccagtag

SEQ ID NO: 131 - DNA, 16 bp, synthetic construct
  source 1..16: mol_type = other DNA
gtcccttcctcacgca

SEQ ID NO: 132 - DNA, 16 bp, synthetic construct
  source 1..16: mol_type = other DNA
caccaaccactcccag

SEQ ID NO: 133 - DNA, 25 bp, synthetic construct
  source 1..25: mol_type = other DNA
cagggaaggagtgcgtggctgcctt

SEQ ID NO: 134 - DNA, 25 bp, synthetic construct
  source 1..25: mol_type = other DNA
gtggttggtgagggtcatctcgctg

SEQ ID NO: 135 - DNA, 22 bp, synthetic construct
  source 1..22: mol_type = other DNA
cagggaaggagtgcgtggctgc

SEQ ID NO: 136 - DNA, 22 bp, synthetic construct
  source 1..22: mol_type = other DNA
gtggttggtgagggtcatctcg

SEQ ID NO: 137 - DNA, 19 bp, synthetic construct
  source 1..19: mol_type = other DNA
cagggaaggagtgcgtggc

SEQ ID NO: 138 - DNA, 19 bp, synthetic construct
  source 1..19: mol_type = other DNA
gtggttggtgagggtcatc

SEQ ID NO: 139 - DNA, 16 bp, synthetic construct
  source 1..16: mol_type = other DNA
cagggaaggagtgcgt

SEQ ID NO: 140 - DNA, 16 bp, synthetic construct
  source 1..16: mol_type = other DNA
gtggttggtgagggtc

SEQ ID NO: 141 - DNA, 25 bp, synthetic construct
  source 1..25: mol_type = other DNA
ttccgtcggtgcgtgaggaagggac

SEQ ID NO: 142 - DNA, 25 bp, synthetic construct
  source 1..25: mol_type = other DNA
gtcgctctactgggagtggttggtg

SEQ ID NO: 143 - DNA, 22 bp, synthetic construct
  source 1..22: mol_type = other DNA
cgtcggtgcgtgaggaagggac

SEQ ID NO: 144 - DNA, 22 bp, synthetic construct
  source 1..22: mol_type = other DNA
gctctactgggagtggttggtg

SEQ ID NO: 145 - DNA, 19 bp, synthetic construct
  source 1..19: mol_type = other DNA
cggtgcgtgaggaagggac

SEQ ID NO: 146 - DNA, 19 bp, synthetic construct
  source 1..19: mol_type = other DNA
ctactgggagtggttggtg

SEQ ID NO: 147 - DNA, 16 bp, synthetic construct
  source 1..16: mol_type = other DNA
tgcgtgaggaagggac

SEQ ID NO: 148 - DNA, 16 bp, synthetic construct
  source 1..16: mol_type = other DNA
ctgggagtggttggtg

SEQ ID NO: 149 - DNA, 33 bp, synthetic construct
  source 1..33: mol_type = other DNA
agatcggaagagcacacgtctgaactccagtca

SEQ ID NO: 150 - DNA, 33 bp, synthetic construct
  source 1..33: mol_type = other DNA
agatcggaagagcgtcgtgtagggaaagagtgt

SEQ ID NO: 151 - DNA, 22 bp, synthetic construct
  source 1..22: mol_type = other DNA
gccttggcacccgagaattcca

SEQ ID NO: 152 - DNA, 33 bp, synthetic construct
  source 1..33: mol_type = other DNA
acactctttccctacacgacgctcttccgatct

SEQ ID NO: 153 - DNA, 32 bp, synthetic construct
  source 1..32: mol_type = other DNA
cactctttccctacacgacgctcttccgatct

SEQ ID NO: 154 - DNA, 37 bp, synthetic construct
  source 1..37: mol_type = other DNA
cggtctcggcattcctgctgaaccgctcttccgatct

SEQ ID NO: 155 - DNA, 31 bp, synthetic construct
  source 1..31: mol_type = other DNA
actctttccctacacgacgctcttccgatct

SEQ ID NO: 156 - DNA, 34 bp, synthetic construct
  source 1..34: mol_type = other DNA
gtgactggagttcagacgtgtgctcttccgatct

SEQ ID NO: 157 - DNA, 21 bp, synthetic construct
  source 1..21: mol_type = other DNA
caagcagaagacggcatacga

SEQ ID NO: 158 - DNA, 23 bp, synthetic construct
  source 1..23: mol_type = other DNA
cgactcactatagggagagcggc

SEQ ID NO: 159 - DNA, 24 bp, synthetic construct
  source 1..24: mol_type = other DNA
aagaacatcgattttccatggcag

SEQ ID NO: 160 - DNA, 33 bp, synthetic construct
  source 1..33: mol_type = other DNA
aacgccaaacctacggctttacttcctgtggct

SEQ ID NO: 161 - DNA, 33 bp, synthetic construct
  source 1..33: mol_type = other DNA
tcttgagtcattcgcagggcatgtgccagacct

SEQ ID NO: 162 - DNA, 33 bp, synthetic construct
  source 1..33: mol_type = other DNA
tcggcgttgtctgctatcgttcttggcactcct

SEQ ID NO: 163 - DNA, 33 bp, synthetic construct
  source 1..33: mol_type = other DNA
ggagcaataaccataaggccgttgacaagccct

SEQ ID NO: 164 - DNA, 33 bp, synthetic construct
  source 1..33: mol_type = other DNA
ggcgtattgccttggttctggcagcctcattgt

SEQ ID NO: 165 - DNA, 33 bp, synthetic construct
  source 1..33: mol_type = other DNA
cagcagagggaacgatttcaacttcctgtggct

SEQ ID NO: 166 - DNA, 33 bp, synthetic construct
  source 1..33: mol_type = other DNA
ctactgcaagggtgtctagaatgtgccagacct

SEQ ID NO: 167 - DNA, 33 bp, synthetic construct
  source 1..33: mol_type = other DNA
gaccgactcgtgaaacgtaatcttggcactcct

SEQ ID NO: 168 - DNA, 33 bp, synthetic construct
  source 1..33: mol_type = other DNA
acacattctttgcgcccagagttgacaagccct

SEQ ID NO: 169 - DNA, 33 bp, synthetic construct
  source 1..33: mol_type = other DNA
atttcattcgacacccggtcgcagcctcattgt

SEQ ID NO: 170 - DNA, 25 bp, synthetic construct
  source 1..25: mol_type = other DNA
acgaccttcttgtagtccttacggc

SEQ ID NO: 171 - DNA, 25 bp, synthetic construct
  source 1..25: mol_type = other DNA
acagtttaggtccactctccaccac

SEQ ID NO: 172 - DNA, 25 bp, synthetic construct
  source 1..25: mol_type = other DNA
tgatagctgaaactagcctcaccgc

SEQ ID NO: 173 - DNA, 25 bp, synthetic construct
  source 1..25: mol_type = other DNA
acccatatcgaggagtcaagttggc

SEQ ID NO: 174 - DNA, 25 bp, synthetic construct
  source 1..25: mol_type = other DNA
atgggctgcctatgccgtaatatcc

SEQ ID NO: 175 - DNA, 25 bp, synthetic construct
  source 1..25: mol_type = other DNA
agtaatgaacagcgcgtggtcacac

SEQ ID NO: 176 - DNA, 29 bp, synthetic construct
  source 1..29: mol_type = other DNA
acaaaggcagccacgcactccttccctgt

SEQ ID NO: 177 - DNA, 29 bp, synthetic construct
  source 1..29: mol_type = other DNA
ctccagcgagatgaccctcaccaaccact

SEQ ID NO: 178 - DNA, 31 bp, synthetic construct
  source 1..31: mol_type = other DNA
gatcggaagagcacacgtctgaactccagtc

SEQ ID NO: 179 - DNA, 29 bp, synthetic construct
  source 1..29: mol_type = other DNA
agtggttggtgagggtcatctcgctggag

SEQ ID NO: 180 - DNA, 63 bp, synthetic construct
  source 1..63: mol_type = other DNA
acaaaggcagccacgcactccttccctgtnnnnnnnnnnnnacactctttccctacacgacgc

SEQ ID NO: 181 - DNA, 62 bp, synthetic construct
  source 1..62: mol_type = other DNA
ctccagcgagatgaccctcaccaaccactnnnnnnnnnnnngtgactggagttcagacgtgt

SEQ ID NO: 182 - DNA, 51 bp, synthetic construct
  source 1..51: mol_type = other DNA
acaaaggcagccacgcactccttccctgtacactctttccctacacgacgc

SEQ ID NO: 183 - DNA, 50 bp, synthetic construct
  source 1..50: mol_type = other DNA
ctccagcgagatgaccctcaccaaccactgtgactggagttcagacgtgt

SEQ ID NO: 184 - DNA, 59 bp, synthetic construct
  source 1..59: mol_type = other DNA
acaaaggcagccacgcactccttccctgtnnnnnnnnacactctttccctacacgacgc

SEQ ID NO: 185 - DNA, 58 bp, synthetic construct
  source 1..58: mol_type = other DNA
ctccagcgagatgaccctcaccaaccactnnnnnnnngtgactggagttcagacgtgt

(19) United States
(12) Patent Application Publication
(10) Pub. No.: US 2024/0035078 A1
(43) Pub. Date: Feb. 1, 2024

(54) METHODS AND COMPOSITIONS FOR AMPLIFYING POLYNUCLEOTIDES

(71) Applicant: Singular Genomics Systems, Inc., San Diego, CA (US)

(72) Inventors: Daan Witters, San Diego, CA (US); Eli N. Glezer, Del Mar, CA (US)

(21) Appl. No.: 18/357,850

(22) Filed: Jul. 24, 2023

Related U.S. Application Data
(60) Provisional application No. 63/392,076, filed on Jul. 25, 2022.

(51) Int. Cl. C12Q 1/686

(52) U.S. Cl. CPC C12Q 1/686

(57) ABSTRACT

Disclosed herein, inter alia, are methods for increasing monoclonal nucleic acid amplification products on a solid support.

### CROSS-REFERENCES TO RELATED APPLICATIONS

This application claims the benefit of U.S. Provisional Application No. 63/392,076, filed Jul. 25, 2022, which is incorporated herein by reference in its entirety and for all purposes.

### SEQUENCE LISTING

The Sequence Listing written in file 051385-583001US_ST26.xml, created Jul. 12, 2023, 168,770 bytes, machine format IBM-PC, MS Windows operating system, is hereby incorporated by reference.

### BACKGROUND

Genetic analysis is taking on increasing importance in modern society as a diagnostic, prognostic, and as a forensic tool. Next generation sequencing (NGS) methods often rely on the amplification of genomic fragments hybridized to polynucleotide primers on a solid surface. Ideally these amplification sites have one initial polynucleotide fragment which is amplified to generate a plurality of identical fragments or complements thereof. However, instances of polyclonal sites, (i.e. sites containing more than one distinct polynucleotide or library molecule) are common and negatively impact sequencing results by increasing sequencing duplications or producing simultaneous and interfering signaling. Furthermore, a potential complication of commercial cluster amplification techniques is that they form a random pattern of clusters on the surface. Thus, there is a need in in the art to improve nucleic acid amplification techniques. Disclosed herein, inter alia, are solutions to these and other problems in the art.

### BRIEF SUMMARY

In an aspect is provided a method of amplifying a polynucleotide on a solid support including a plurality of immobilized primers, the method including hybridizing a second platform primer binding sequence of a first immobilized polynucleotide to a second immobilized primer; wherein the first immobilized polynucleotide includes a first platform primer sequence immobilized to a solid support, a template sequence, and the second platform primer binding sequence; hybridizing a third platform primer binding sequence of a second immobilized polynucleotide to a third immobilized primer including a cleavable site; wherein the second immobilized polynucleotide includes the first platform primer sequence, a template sequence, and the third platform primer binding sequence; extending the second immobilized primer with a polymerase to form a first amplification product and extending the third immobilized primer with a polymerase to form a second amplification product including the cleavable site; cleaving the cleavable site and removing the second amplification product; and amplifying the first amplification product and the first immobilized polynucleotide.

In another aspect is provided a method of forming a first immobilized polynucleotide and a second immobilized polynucleotide on a solid support, the method including: contacting a solid support with a first polynucleotide and a second polynucleotide, wherein the solid support includes a population of first platform primers, a population of second platform primers, and a population of third platform primers, wherein each third platform primer includes a cleavable site and wherein each of the first platform primers, the second platform primers and the third platform primers are immobilized to the solid support; hybridizing a first platform primer binding sequence of the first polynucleotide to one of the first platform primers, wherein the first polynucleotide includes the first platform primer binding sequence, a template sequence, and a second platform primer sequence; hybridizing a first platform primer binding sequence of the second polynucleotide to one of the first platform primers, wherein the second polynucleotide includes the first platform primer binding sequence, a template sequence, and a third platform primer sequence; extending the first platform primer with a polymerase to form the first immobilized polynucleotide including the first platform primer sequence, a complement of the template sequence, and a second platform primer binding sequence.

In an aspect is provided a solid support including a plurality of amplification sites, wherein each amplification site includes a population of first platform primers, a population of second platform primers, and a population of third platform primers, wherein each of the third platform primers include a cleavable site. In embodiments, each of the first and second platform primers do not include a cleavable site.

In an aspect is provided a kit including a solid support including a plurality of amplification sites, wherein each amplification site includes a population of first platform primers, a population of second platform primers, and a population of third platform primers, wherein each of the third platform primers include a cleavable site.

### BRIEF DESCRIPTION OF THE DRAWINGS

FIG. 1 shows examples of the adapter oligonucleotide sequences, referred to as P1, P2, and P3 adapters, respectively. The P1 adapter contains a first platform primer sequence (pp1), which is a sequence complementary to a first immobilized primer (e.g., an oligonucleotide attached to a solid support), an optional index sequence, and a region complementary to a first sequencing primer (SP1). The P2 adapter contains a second platform primer sequence (pp2), which is a sequence complementary to a second immobilized primer, an optional index sequence, and a region complementary to a second sequencing primer (SP2). The P3 adapter contains a third platform primer 3 (pp3), which is a sequence complementary to a third immobilized primer, an optional index sequence, and a region complementary to a third sequencing primer (SP3). The dashed lines are indicative of regions within the adapter and are included to aid the eye in the different arrangement of the sequences and are not indicative of the overall size/length (i.e., the index sequence may not be the same length as the sequencing primer despite the illustration showing the index sequence and sequencing primer as being the same size. In embodiments, the 5′ end of the adapter includes the platform primer sequence.

FIGS. 2A-2B shows an example of the library of DNA molecules prepared according to an embodiment of the methods described herein, wherein adapters are ligated to the sample polynucleotides. Following standard library prep protocols (e.g., fragmenting, repairing, A-tailing), a reaction mixture containing different adapters (e.g., P1, P2, and P3, and/or the complements thereof) are mixed together with nucleic acid molecules. FIG. 2A shows a DNA template with P1 and P2′ adapters ligated to the ends when hybridized together (top), and the subsequent amplification products (bottom). FIG. 2B shows a DNA template with P1 and P3′ adapters ligated to the ends when hybridized together (top) and the subsequent amplification products (bottom). It is understood that color, if observable in the Figure, is not an indication of a different sequence; for example, the SP1 sequence of one color may be similar or substantially identical to the SP1 sequence of a different color. As illustrated, two Y-shaped adapters are ligated to the sample polynucleotide, however it is understood that alternative shaped adapters are contemplated herein (e.g., hairpin adapters, blunt end adapters, bubble adapters, and the like). In embodiments, each end of the sample polynucleotide is ligated to adapters having the same shape (e.g., both ends include a Y-adapter). In embodiments, each end of the sample polynucleotide is ligated to adapters having different shapes (e.g., the first adapter is a Y adapter and the second adapter is a hairpin adapter).

FIG. 3. Illustrated in FIG. 3 is a pattered solid support containing a plurality of features. Each feature includes a plurality of immobilized oligonucleotides, referred to as platform primer oligonucleotides. Within each feature, as depicted in FIG. 3, the plurality of immobilized oligonucleotides include a first platform primer oligonucleotide (pp1) having complementarity to all or a portion of P1, a second platform primer oligonucleotide (pp2) having complementarity to all or a portion of P2, and a third platform primer oligonucleotide (pp3) having complementarity to all or a portion of P3. In embodiments, each feature includes a plurality of immobilized oligonucleotides. In embodiments, the plurality includes include a first population of platform primer oligonucleotides (pp1) having complementarity to all or a portion of P1, or the complement thereof; a second population of platform primer oligonucleotides (pp2) having complementarity to all or a portion of P2, or the complement thereof; and a third population of platform primer oligonucleotides (pp3) having complementarity to all or a portion of P3, or the complement thereof. The third platform primer oligonucleotides includes one or more cleavable sites, depicted as the plaque shape in FIG. 3.

FIGS. 4A-4F. Seeding and amplification of library molecules. The prepared library molecules are allowed to contact the solid support and 0, 1, 2, or more molecules may contact a single feature. For example, if one molecule seeds (i.e., hybridizes to the surface-immobilized oligonucleotide) a single feature and is amplified it is referred to as a monoclonal colony. Monoclonal colony formation for a P1′-template-P2 molecule is illustrated in FIGS. 4A-4C, where an initial molecule anneals to a first surface-immobilized oligonucleotide and is extended to form an immobilized extension product. The initial molecule is removed and the immobilized extension product hybridizes to a second surface-immobilized oligonucleotide, and with a polymerase is extended to form a second immobilized extension product (FIG. 4B). Under suitable amplification conditions, the process is repeated to form a plurality of immobilized extension product, as illustrated in FIG. 4E. A similar process occurs for P1′-template-P3 molecules to generate a monoclonal colony in a feature (FIG. 4C-4D), of which the final product is exemplified in FIG. 4F.

FIGS. 5A-5D. Reducing polyclonality in a feature. FIG. 5A illustrates seeding and extension of two molecules, a P1′-template-P2 molecule (left) and a P1′-template-P3 molecule (right). In embodiments, the third platform primer oligonucleotides (i.e., pp3) includes one or more cleavable sites, depicted as the plaque shape. An additional round of extension, whereby the immobilized extension products anneal and to another surface-immobilized oligonucleotide (FIG. 5B), and with a polymerase is extended to form additional immobilized extension products (FIG. 5C). The cleavable site on the platform primer oligonucleotides does not preclude hybridization or extension. The surface-immobilized oligonucleotides and extension products including a cleavable site are cleaved and additional rounds of amplification (FIG. 5D) are performed to enable the P1-template-P2′ containing amplification products to dominate the feature. Cleaving the cleavable site prevents extension of the cleaved primers by a polymerase, but hybridization is still permitted.

FIG. 6A-6B. Array with reduced polyclonality. FIG. 6A depicts a 4×6 patterned array following an initial seeding event (i.e., wherein a plurality of library molecules contact the solid support). The outcome of seeding at an equal ratio of molecules to available sites, referred to as 1:1 seeding, estimates about 37% of the available sites will be empty (empty circles), about 37% of the available sites are contacted by a single molecule (solid color circles), about 18% hybridize two molecules (represented as a circle containing two different colors with equal proportion), and about 8% contain three or more different molecules (represented as a circle containing two different colors with unequal proportion). FIG. 6B illustrates the reduction in polyclonality following the method described herein.

### DETAILED DESCRIPTION

The aspects and embodiments described herein relate to increasing the number of detectable clusters of polynucleotides on a solid support.

### I. Definitions

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the term “control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As used herein, the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association.

As used herein, the term “complementary” or “substantially complementary” refers to the hybridization, base pairing, or the formation of a duplex between nucleotides or nucleic acids. For example, complementarity exists between the two strands of a double-stranded DNA molecule or between an oligonucleotide primer and a primer binding site on a single-stranded nucleic acid when a nucleotide (e.g., RNA or DNA) or a sequence of nucleotides is capable of base pairing with a respective cognate nucleotide or cognate sequence of nucleotides. As described herein and commonly known in the art the complementary (matching) nucleotide of adenosine (A) is thymidine (T) and the complementary (matching) nucleotide of guanosine (G) is cytosine (C). Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. “Duplex” means at least two oligonucleotides and/or polynucleotides that are fully or partially complementary undergo Watson-Crick type base pairing among all or most of their nucleotides so that a stable complex is formed. In embodiments, a first template polynucleotide and a second template polynucleotide of an overlapping cluster are not substantially complementary (e.g., are at least 50%, 75%, 90%, or more non-complementary to each other).

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin or loop structure, with or without an overhang) or portions of separate polynucleotides. In embodiments, one or both sequences in a pair of complementary sequences form portions of longer polynucleotides, which may or may not include additional regions of complementarity.

As used herein, the term “contacting” is used in accordance with its plain ordinary meaning and refers to the process of allowing at least two distinct species (e.g., chemical compounds including biomolecules or cells) to become sufficiently proximal to react, interact or physically touch. However, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound, nucleic acid, a protein, or enzyme (e.g., a DNA polymerase).

As may be used herein, the terms “nucleic acid,” “nucleic acid molecule,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. In some embodiments, an oligonucleotide is a primer configured for extension by a polymerase when the primer is annealed completely or partially to a complementary nucleic acid template. A primer is often a single stranded nucleic acid. In certain embodiments, a primer, or portion thereof, is substantially complementary to a portion of an adapter. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In some embodiments, an oligonucleotide may be immobilized to a solid support.

Two or more associated species are “tethered”, “coated”, “attached”, or “immobilized” to one another or to a common solid or semisolid support (e.g. a receiving substrate). An association may refer to a relationship, or connection, between two entities. As used herein, an “immobilized polynucleotide” or an “immobilized primer” refers to a polynucleotide or a primer that is attached to a solid surface, such as a solid support. The immobilized polynucleotide and/or immobilized primer may be attached covalently (e.g. through a linker) or non-covalently to a solid support. In embodiments, immobilized polynucleotide and/or immobilized primer is covalently attached to a solid support.

As used herein, the terms “library”, “RNA library” or “DNA library” or “library of DNA molecules” are used in accordance with their plain ordinary meaning and refer to a collection or a population of similarly sized nucleic acid fragments with known adapter sequences (e.g., known adapters attached to the 5′ and 3′ ends of each of the fragments). In embodiments, the library includes a plurality of nucleic acid fragments including one or more adapter sequences. In embodiments, the library includes circular nucleic acid templates. Libraries are typically prepared from input RNA, DNA, or cDNA and are processed by fragmentation, size selection, end-repair, adapter ligation, amplification, and purification. Alternative amplification-free (i.e., PCR free) methods for preparing a library of molecules include shearing input polynucleotides, size selecting and ligating adapters. A library may correspond to a single sample or a single origin. Multiple libraries, each with their own unique adapter sequences, may be pooled and sequenced in the same sequencing run using the methods described herein.

As used herein, the terms “polynucleotide primer” and “primer” refers to any polynucleotide molecule that may hybridize to a polynucleotide template, be bound by a polymerase, and be extended in a template-directed process for nucleic acid synthesis. The primer may be a separate polynucleotide from the polynucleotide template, or both may be portions of the same polynucleotide (e.g., as in a hairpin structure having a 3′ end that is extended along another portion of the polynucleotide to extend a double-stranded portion of the hairpin). Primers (e.g., forward or reverse primers) may be attached to a solid support. A primer can be of any length depending on the particular technique it will be used for. For example, PCR primers are generally between 10 and 40 nucleotides in length. The length and complexity of the nucleic acid fixed onto the nucleic acid template may vary. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide. A “primer” is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

As used herein, a “platform primer” is a primer oligonucleotide immobilized or otherwise bound to a solid support (i.e. an immobilized oligonucleotide). Examples of platform primers include P7 and P5 primers, or S1 and S2 sequences, or the reverse complements thereof. A “platform primer binding sequence” refers to a sequence or portion of an oligonucleotide that is capable of binding to a platform primer (e.g., the platform primer binding sequence is complementary to the platform primer). In embodiments, a platform primer binding sequence may form part of an adapter. In embodiments, a platform primer binding sequence is complementary to a platform primer sequence. In embodiments, a platform primer binding sequence is complementary to a primer.

As used herein, the terms “solid support” and “substrate” and “solid surface” are used interchangeably and refers to discrete solid or semi-solid surfaces to which a plurality of nucleic acid (e.g., primers) may be attached. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A solid support may comprise a discrete particle that may be spherical (e.g., microspheres) or have a non-spherical or irregular shape, such as cubic, cuboid, pyramidal, cylindrical, conical, oblong, or disc-shaped, and the like. Solid supports may be in the form of discrete particles, which alone does not imply or require any particular shape. The term “particle” means a small body made of a rigid or semi-rigid material. The body can have a shape characterized, for example, as a sphere, oval, microsphere, or other recognized particle shape whether having regular or irregular dimensions. As used herein, the term “discrete particles” refers to physically distinct particles having discernible boundaries. The term “particle” does not indicate any particular shape. The shapes and sizes of a collection of particles may be different or about the same (e.g., within a desired range of dimensions, or having a desired average or minimum dimension). A particle may be substantially spherical (e.g., microspheres) or have a non-spherical or irregular shape, such as cubic, cuboid, pyramidal, cylindrical, conical, oblong, or disc-shaped, and the like. In embodiments, the particle has the shape of a sphere, cylinder, spherocylinder, or ellipsoid. Discrete particles collected in a container and contacting one another will define a bulk volume containing the particles, and will typically leave some internal fraction of that bulk volume unoccupied by the particles, even when packed closely together. In embodiments, cores and/or core-shell particles are approximately spherical. As used herein the term “spherical” refers to structures which appear substantially or generally of spherical shape to the human eye, and does not require a sphere to a mathematical standard. In other words, “spherical” cores or particles are generally spheroidal in the sense of resembling or approximating to a sphere. In embodiments, the diameter of a spherical core or particle is substantially uniform, e.g., about the same at any point, but may contain imperfections, such as deviations of up to 1, 2, 3, 4, 5 or up to 10%. Because cores or particles may deviate from a perfect sphere, the term “diameter” refers to the longest dimension of a given core or particle. Likewise, polymer shells are not necessarily of perfect uniform thickness all around a given core. Thus, the term “thickness” in relation to a polymer structure (e.g., a shell polymer of a core-shell particle) refers to the average thickness of the polymer layer.

A solid support may further comprise a polymer or hydrogel on the surface to which the primers are attached (e.g., the primers are covalently attached to the polymer, wherein the polymer is in direct contact with the solid support). Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor, silica or silica-based materials including silicon and modified silicon, carbon, metals, inorganic glasses, optical fiber bundles, photopatternable dry film resists, UV-cured adhesives and polymers. The solid supports for some embodiments have at least one surface located within a flow cell. The solid support, or regions thereof, can be substantially flat. The solid support can have surface features such as wells, pits, channels, ridges, raised regions, pegs, posts or the like. The term solid support is encompassing of a substrate (e.g., a flow cell) having a surface comprising a polymer coating covalently attached thereto.

In embodiments, the solid support is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In certain embodiments a substrate comprises a surface (e.g., a surface of a flow cell, a surface of a tube, a surface of a chip), for example a metal surface (e.g., steel, gold, silver, aluminum, silicon and copper). In some embodiments a substrate (e.g., a substrate surface) is coated and/or comprises functional groups and/or inert materials.

In certain embodiments a substrate comprises a bead, a chip, a capillary, a plate, a membrane, a wafer (e.g., silicon wafers), a comb, or a pin for example. In some embodiments a substrate comprises a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, glass, nylon, Wang resin, Merrifield resin, metal (e.g., iron, a metal alloy, sepharose, agarose, polyacrylamide, dextran, cellulose and the like or combinations thereof. In some embodiments a substrate comprises a magnetic material (e.g., iron, nickel, cobalt, platinum, aluminum, and the like). In certain embodiments a substrate comprises a magnetic bead (e.g., DYNABEADS®, hematite, AMPure XP). Magnets can be used to purify and/or capture nucleic acids bound to certain substrates (e.g., substrates comprising a metal or magnetic material).

The terms “particle” and “bead” are used interchangeably and mean a small body made of a rigid or semi-rigid material. The body can have a shape characterized, for example, as a sphere, oval, microsphere, or other recognized particle shape whether having regular or irregular dimensions. A “nanoparticle,” as used herein, is a particle wherein the longest diameter is less than or equal to 1000 nanometers. Nanoparticles may be composed of any appropriate material. For example, nanoparticle cores may include appropriate metals and metal oxides thereof (e.g., a metal nanoparticle core), carbon (e.g., an organic nanoparticle core) silicon and oxides thereof (e.g., a silicon nanoparticle core) or boron and oxides thereof (e.g., a boron nanoparticle core), or mixtures thereof. Nanoparticles may be composed of at least two distinct materials, one material (e.g., silica) forms the core and the other material forms the shell (e.g., copolymer) surrounding the core.

In embodiments, the solid support is a multi-well container. In embodiments, the solid support is a plate. The term “multi-well container” or “plate” as used herein, refers to a substrate comprising a surface, the surface including a plurality of reaction chambers separated from each other by interstitial regions on the surface. In embodiments, the microplate has dimensions as provided and described by American National Standards Institute (ANSI) and Society for Laboratory Automation And Screening (SLAS); for example the tolerances and dimensions set forth in ANSI SLAS 1-2004 (R2012); ANSI SLAS 2-2004 (R2012); ANSI SLAS 3-2004 (R2012); ANSI SLAS 4-2004 (R2012); and ANSI SLAS 6-2012, which are incorporated herein by reference.

In embodiments, the solid support is an unpatterned solid support. The term “unpatterned solid support” as used herein refers to a solid support with a uniform polymer surface including, for example, amplification primers randomly distributed throughout the polymer surface. This is in contrast to a patterned solid support, wherein amplification primers, for example, as localized to specific regions of the surface, such as to wells in an array. In embodiments, an unpatterned solid support does not include organized surface features such as wells, pits, channels, ridges, raised regions, pegs, posts or the like. In embodiments, the surface of an unpatterned solid support does not contain interstitial regions. In embodiments, an unpatterned solid support includes a polymer (e.g., a hydrophilic polymer). In certain embodiments, the unpatterned solid support includes a plurality of oligonucleotides (e.g., primer oligonucleotides) randomly distributed throughout the polymer (e.g., the plurality of primer oligonucleotides are covalently attached to the polymer in a random distribution, as illustrated in FIGS. 8D-8F). An unpatterned solid support may be, for example, a glass slide including a polymer coating (a hydrophilic polymer coating).

As used herein, the term “channel” refers to a passage in or on a substrate material that directs the flow of a fluid. A channel may run along the surface of a substrate, or may run through the substrate between openings in the substrate. A channel can have a cross section that is partially or fully surrounded by substrate material (e.g., a fluid impermeable substrate material). For example, a partially surrounded cross section can be a groove, trough, furrow or gutter that inhibits lateral flow of a fluid. The transverse cross section of an open channel can be, for example, U-shaped, V-shaped, curved, angular, polygonal, or hyperbolic. A channel can have a fully surrounded cross section such as a tunnel, tube, or pipe. A fully surrounded channel can have a rounded, circular, elliptical, square, rectangular, or polygonal cross section. A microfluidic flow channel is characterized by cross-sectional dimensions less than 1000 microns. Usually at least one, and preferably all, cross-sectional dimensions are greater than 500 microns.

As used herein, the term “polymer” refers to macromolecules having one or more structurally unique repeating units. The repeating units are referred to as “monomers,” which are polymerized for the polymer. Typically, a polymer is formed by monomers linked in a chain-like structure. A polymer formed entirely from a single type of monomer is referred to as a “homopolymer.” A polymer formed from two or more unique repeating structural units may be referred to as a “copolymer.” A polymer may be linear or branched, and may be random, block, polymer brush, hyperbranched polymer, bottlebrush polymer, dendritic polymer, or polymer micelles. The term “polymer” includes homopolymers, copolymers, tripolymers, tetra polymers and other polymeric molecules made from monomeric subunits. Copolymers include alternating copolymers, periodic copolymers, statistical copolymers, random copolymers, block copolymers, linear copolymers and branched copolymers. The term “polymerizable monomer” is used in accordance with its meaning in the art of polymer chemistry and refers to a compound that may covalently bind chemically to other monomer molecules (such as other polymerizable monomers that are the same or different) to form a polymer.

Polymers can be hydrophilic, hydrophobic, or amphiphilic, as known in the art. Thus, “hydrophilic polymers” are substantially miscible with water and include, but are not limited to, polyethylene glycol and the like. “Hydrophobic polymers” are substantially immiscible with water and include, but are not limited to, polyethylene, polypropylene, polybutadiene, polystyrene, polymers disclosed herein, and the like. “Amphiphilic polymers” have both hydrophilic and hydrophobic properties and are typically copolymers having hydrophilic segment(s) and hydrophobic segment(s). Polymers include homopolymers, random copolymers, and block copolymers, as known in the art. The term “homopolymer” refers, in the usual and customary sense, to a polymer having a single monomeric unit. The term “copolymer” refers to a polymer derived from two or more monomeric species. The term “random copolymer” refers to a polymer derived from two or more monomeric species with no preferred ordering of the monomeric species. The term “block copolymer” refers to polymers having two or homopolymer subunits linked by covalent bond. Thus, the term “hydrophobic homopolymer” refers to a homopolymer which is hydrophobic. The term “hydrophobic block copolymer” refers to two or more homopolymer subunits linked by covalent bonds and which is hydrophobic.

As used herein, the term “hydrogel” refers to a three-dimensional polymeric structure that is substantially insoluble in water, but which is capable of absorbing and retaining large quantities of water to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can be comprised of natural or synthetic polymers. In some embodiments, the hydrogel polymer includes 60-90% fluid, such as water, and 10-30% polymer. In certain embodiments, the water content of hydrogel is about 70-80%.

Hydrogels may be prepared by cross-linking hydrophilic biopolymers or synthetic polymers. Thus, in some embodiments, the hydrogel may include a crosslinker. As used herein, the term “crosslinker” refers to a molecule that can form a three-dimensional network when reacted with the appropriate base monomers. Examples of the hydrogel polymers, which may include one or more crosslinkers, include but are not limited to, hyaluronans, chitosans, agar, heparin, sulfate, cellulose, alginates (including alginate sulfate), collagen, dextrans (including dextran sulfate), pectin, carrageenan, polylysine, gelatins (including gelatin type A), agarose, (meth)acrylate-oligolactide-PEO-oligolactide-(meth)acrylate, PEO-PPO-PEO copolymers (Pluronics), poly(phosphazene), poly(methacrylates), poly(N-vinylpyrrolidone), PL(G)A-PEO-PL(G)A copolymers, poly(ethylene imine), polyethylene glycol (PEG)-thiol, PEG-acrylate, acrylamide, N,N′-bis(acryloyl)cystamine, PEG, polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), bisacrylamide, diacrylate, diallylamine, triallylamine, divinyl sulfone, diethyleneglycol diallyl ether, ethyleneglycol diacrylate, polymethyleneglycol diacrylate, polyethyleneglycol diacrylate, trimethylopropoane trimethacrylate, ethoxylated trimethylol triacrylate, or ethoxylated pentaerythritol tetracrylate, or combinations thereof. Thus, for example, a combination may include a polymer and a crosslinker, for example polyethylene glycol (PEG)-thiol/PEG-acrylate, acrylamide/N,N′-bis(acryloyl)cystamine (BACy), or PEG/polypropylene oxide (PPO).

The term “surface” is intended to mean an external part or external layer of a substrate. The surface can be in contact with another material such as a gas, liquid, gel, polymer, organic polymer, second surface of a similar or different material, metal, or coat. The surface, or regions thereof, can be substantially flat. The substrate and/or the surface can have surface features such as wells, pits, channels, ridges, raised regions, pegs, posts or the like.

As used herein, the terms “cluster” and “colony” are used interchangeably to refer to a site (e.g., a discrete site) on a solid support that includes a plurality of immobilized polynucleotides and a plurality of immobilized complementary polynucleotides. In embodiments, the polynucleotides consist of amplicons of a single species (e.g., “monoclonal”), thereby forming a homogenous cluster. However, in preferred embodiments, the polynucleotides at a given site are heterogeneous (e.g., “polyclonal”), such that individual molecules having different sequences are present at the site or feature. In some embodiments, a polyclonal cluster includes template polynucleotides including the same template sequence but containing different adapter sequences compared to other substantially identical template polynucleotides (e.g., the same target polynucleotide sequence from different samples, prepared with the different adapter sequences). The term “clustered array” refers to an array formed from such clusters or colonies. In this context the term “array” is not to be understood as requiring an ordered arrangement of clusters. The term “array” is used in accordance with its ordinary meaning in the art and refers to a population of different molecules that are attached to one or more solid-phase substrates such that different molecules can be differentiated from each other according to their relative location. An array can include different molecules that are each located at different addressable features on a solid-phase substrate. In some embodiments, an array of sites is provided, wherein each of a plurality of the sites includes a first nucleic acid template and a second nucleic acid template and wherein the first nucleic acid template has a sequence that is different from the sequence of the second nucleic acid template. There can be greater than two different templates (e.g., greater than three different templates, greater than four different templates, etc.) at each of a plurality of sites, in some embodiments. The molecules of the array can be nucleic acid primers, nucleic acid probes, nucleic acid templates, or nucleic acid enzymes such as polymerases or ligases. Arrays useful in embodiments can have densities that range from about 2 different features to many millions, billions, or higher. The density of an array can be from 2 to as many as a billion or more different features per square cm. For example, an array can have at least about 100 features/cm², at least about 1,000 features/cm², at least about 10,000 features/cm², at least about 100,000 features/cm², at least about 10,000,000 features/cm², at least about 100,000,000 features/cm², at least about 1,000,000,000 features/cm², at least about 2,000,000,000 features/cm² or higher. In embodiments, the arrays have features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher. In some embodiments, an amplification site is referred to as “monoclonal” or “substantially monoclonal” if it includes sufficiently few polyclonal contaminants to produce a detectable signal in any method of nucleic acid analysis that is influenced by the sequence of the template. For example, a “monoclonal” population of polynucleotides can include any population that produces a signal (e.g., a sequencing signal, a nucleotide incorporation signal) that can be detected using a particular sequencing system.

As used herein, the term “amplification site” refers to a location (e.g., a discrete site) on a solid support wherein amplification of a polynucleotide may occur or has occurred. An amplification site may be on a solid support that includes a plurality of immobilized polynucleotides, and a plurality of immobilized complementary polynucleotides. In embodiments, an amplification cluster can be generated at or on this amplification site wherein multiple template polynucleotides are immobilized within one spot of an array and subsequently amplified. An amplification site can contain only a single immobilized polynucleotide or it can contain a population of several immobilized polynucleotides. In some embodiments, an amplification site can include multiple different immobilized polynucleotide species, each species being present in one or more copies. Amplification sites of an array are typically discrete. The discrete sites can be contiguous, or they can have spaces (e.g., interstitial spaces) between each other. In embodiments, the same template polynucleotide sequence may be present in the same location (e.g., same x-y coordinates and/or physical location, such as the same well). In embodiments, the same template polynucleotide sequence may be present in different locations (e.g., different x-y coordinates and/or physical location) within the same amplification site (e.g., a plurality of amplification products that have the same template polynucleotide sequence are within the same amplification site). In embodiments, multiple template polynucleotides seed one spot (i.e., a feature) of a patterned array or unpatterned solid support. In embodiments, a fraction of the surface area within the feature is occupied by copies of one template, and another fraction of the patterned spot can be occupied by copies of another template. In embodiments, the term “monoclonal” and its variants is used to describe a population of polynucleotides where a substantial portion of the members of the population (e.g., at least about 50%, typically at least 75%, 80%, 85%, 90%, 95%, or 99%) share at least 80% identity of the nucleotide sequence. Typically, at least about 90% of the population, typically at least about 95%, more typically at least about 99%, 99.5% or 99.9%) are generated via amplification or template-dependent replication of a polynucleotide sequence, which is present amongst a substantial portion of members of the monoclonal polynucleotide population. All members of a monoclonal population need not be completely identical or complementary to each other. For example, different portions of a polynucleotide template can become amplified or replicated to produce the members of the resulting monoclonal population; similarly, one or more amplification errors and/or incomplete extensions may occur during amplification of the original template, thereby generating a monoclonal population whose individual members can exhibit sequence variability amongst themselves. In embodiments, “substantially monoclonal” when used in reference to one or more polynucleotide populations, refers to one or more polynucleotide populations of polynucleotides that are at least 80% identical to the original single template used as a basis for clonal amplification to produce the substantially monoclonal population.

Detection can be carried out at ensemble or single molecule levels on an array. Ensemble level detection is detection that occurs in a way that several copies of a single template sequence (e.g. multiple amplicons of a template) are detected at each individual site and individual copies at the site are not distinguished from each other. Thus, ensemble detection provides an average signal from many copies of a particular template sequence at the site. For example, the site can contain at least 10, 100, 1000 or more copies of a particular template sequence. Of course, a site can contain multiple different template sequences each of which is present as an ensemble. Alternatively, detection at a single molecule level includes detection that occurs in a way that individual template sequences are individually resolved on the array, each at a different site. Thus, single molecule detection provides a signal from an individual molecule that is distinguished from one or more signals that may arise from a population of molecules within which the individual molecule is present. Of course, even in a single molecule array, a site can contain several different template sequences (e.g., two or more template sequence regions located along a single nucleic acid molecule).

An array of sites (e.g., an array of features) can appear as a grid of spots or patches. The sites can be located in a repeating pattern or in an irregular non-repeating pattern. Particularly useful patterns are hexagonal patterns, rectilinear patterns, grid patterns, patterns having reflective symmetry, patterns having rotational symmetry, or the like. Asymmetric patterns can also be useful; in embodiments, the array of features are present in an asymmetric pattern.

The size of the sites and/or spacing between the sites in an array can vary to achieve high density, medium density, or lower density. High density arrays are characterized as having sites with a pitch that is less than about 15 m. Medium density arrays have sites with a pitch that is about 15 to 30 μm, while low density arrays have a pitch that is greater than 30 μm. An array useful in some embodiments can have sites with a pitch that is less than 100 μm, 50 μm, 10 μm, 5 μm, 1 μm, or 0.5 μm. An embodiment of the methods set forth herein can be used to image an array at a resolution sufficient to distinguish sites at the above densities or density ranges. However, the detecting step will typically use a detector having a spatial resolution that is too low to resolve points at a distance equivalent to the spacing between a first template (or first primer extension product hybridized thereto) and a second template (or second primer extension product hybridized thereto) of an overlapping cluster at an individual site. In particular embodiments, sites of an array can each have an area that is larger than about 100 nm², 250 nm², 500 nm², 1 μm², 2.5 μm², 5 μm², 10 μm², 100 μm², or 500 μm². Alternatively or additionally, sites of an array can each have an area that is smaller than about 1 mm², 500 μm², 100 μm², 25 μm², 10 μm², 5 μm², 1 μm², 500 nm², or 100 nm². Indeed, a site can have a size that is in a range between an upper and lower limit selected from those exemplified above.

Generally, an array will have sites with different nucleic acid sequence content. In embodiments, each of a plurality of sites of the array contains different ratios of a population of template polynucleotides, wherein each population of template polynucleotides contains different sequencing primer binding sites. Accordingly, each of the sites in an array can contain a nucleic acid sequence that is unique compared to the nucleic acid sequences at the other sites in the array. However, in some cases an array can have redundancy such that two or more sites have the same nucleic acid content.

As used herein, the term “each,” when used in reference to a collection of items, is intended to identify an individual item in the collection but does not necessarily refer to every item in the collection. Exceptions can occur if explicit disclosure or context clearly dictates otherwise.

Nucleic acids, including e.g., nucleic acids with a phosphorothioate backbone, can include one or more reactive moieties. As used herein, the term “reactive moiety” includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

As used herein, the term “template polynucleotide” or “template sequence” refers to any polynucleotide molecule that may be bound by a polymerase and utilized as a template for nucleic acid synthesis. A template polynucleotide may refer to the sequence of polynucleotides or a complement thereof. A template polynucleotide may be a target polynucleotide. In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. In general, the term “target sequence” refers to a nucleic acid sequence on a single strand of nucleic acid. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target polynucleotide in a reaction with the corresponding primer polynucleotide(s). In the context of selective sequencing, “target polynucleotide(s)” refers to the subset of polynucleotide(s) to be sequenced from within a starting population of polynucleotides.

In embodiments, a target polynucleotide is a cell-free polynucleotide. In general, the terms “cell-free,” “circulating,” and “extracellular” as applied to polynucleotides (e.g. “cell-free DNA” (cfDNA) and “cell-free RNA” (cfRNA)) are used interchangeably to refer to polynucleotides present in a sample from a subject or portion thereof that can be isolated or otherwise manipulated without applying a lysis step to the sample as originally collected (e.g., as in extraction from cells or viruses). Cell-free polynucleotides are thus unencapsulated or “free” from the cells or viruses from which they originate, even before a sample of the subject is collected. Cell-free polynucleotides may be produced as a byproduct of cell death (e.g. apoptosis or necrosis) or cell shedding, releasing polynucleotides into surrounding body fluids or into circulation. Accordingly, cell-free polynucleotides may be isolated from a non-cellular fraction of blood (e.g. serum or plasma), from other bodily fluids (e.g. urine), or from non-cellular fractions of other types of samples.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the terms “analogue” and “analog”, in reference to a chemical compound, refers to compound having a structure similar to that of another one, but differing from it in respect of one or more different atoms, functional groups, or substructures that are replaced with one or more other atoms, functional groups, or substructures. In the context of a nucleotide, a nucleotide analog refers to a compound that, like the nucleotide of which it is an analog, can be incorporated into a nucleic acid molecule (e.g., an extension product) by a suitable polymerase, for example, a DNA polymerase in the context of a nucleotide analogue. The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, e.g., see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, a “native” nucleotide is used in accordance with its plain and ordinary meaning and refers to a naturally occurring nucleotide that does not include an exogenous label (e.g., a fluorescent dye, or other label) or chemical modification such as may characterize a nucleotide analog (e.g., a reversible terminating moiety). Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate). A “canonical” nucleotide is an unmodified nucleotide.

As used herein, the term “modified nucleotide” refers to nucleotide modified in some manner. Typically, a nucleotide contains a single 5-carbon sugar moiety, a single nitrogenous base moiety and 1 to three phosphate moieties. In embodiments, a nucleotide can include a blocking moiety (alternatively referred to herein as a reversible terminator moiety) and/or a label moiety. A blocking moiety (e.g., a reversible terminator) on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. In embodiments, the blocking moiety is attached to the 3′ oxygen of the nucleotide and is independently —NH₂, —CN, —CH₃, C₂-C₆ allyl (e.g., —CH₂—CH═CH₂), methoxyalkyl (e.g., —CH₂—O—CH₃), or —CH₂N₃. In embodiments, the blocking moiety is attached to the 3′ oxygen of the nucleotide and is independently

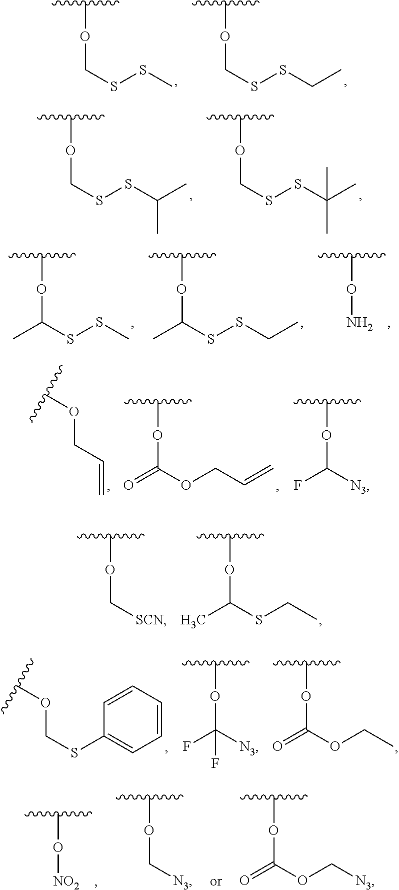

wherein the 3′ oxygen of the nucleotide is explicitly shown in the formulae above. A label moiety of a nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both. Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the OH group at the 3′-position of deoxyribose. Nucleotide analogues and DNA polymerase-based DNA sequencing are also described in U.S. Pat. No. 6,664,079, which is incorporated herein by reference in its entirety for all purposes. Non-limiting examples of detectable labels include labels comprising fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, the label is a fluorophore.

In embodiments, the nucleotides of the present disclosure use a cleavable linker to attach the label to the nucleotide. The use of a cleavable linker ensures that the label can, if required, be removed after detection, avoiding any interfering signal with any labelled nucleotide incorporated subsequently. The use of the term “cleavable linker” is not meant to imply that the whole linker is required to be removed from the nucleotide base. The cleavage site can be located at a position on the linker that ensures that part of the linker remains attached to the nucleotide base after cleavage. The linker can be attached at any position on the nucleotide base provided that Watson-Crick base pairing can still be carried out. In the context of purine bases, it is preferred if the linker is attached via the 7-position of the purine or the preferred deazapurine analogue, via an 8-modified purine, via an N-6 modified adenosine or an N-2 modified guanine. For pyrimidines, attachment is preferably via the 5-position on cytidine, thymidine or uracil and the N-4 position on cytosine.

The term “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), or hydrazine (N₂H₄)). A chemically cleavable linker is non-enzymatically cleavable. In embodiments, the cleavable linker is cleaved by contacting the cleavable linker with a cleaving agent. In embodiments, the cleaving agent is a phosphine containing reagent (e.g., TCEP or THPP), sodium dithionite (Na₂S₂O₄), weak acid, hydrazine (N₂H₄), Pd(0), or light-irradiation (e.g., ultraviolet radiation). In embodiments, cleaving includes removing. For clarity, the terms “cleavable linker” and “cleavable site” are different terms with different meanings as used herein. For example, a cleavable linker may include a covalent linker that includes one or more cleavable sites.

A “cleavable site” or “scissile linkage” in the context of a polynucleotide including a cleavable site (or scissile linkage) is a site on the polynucleotide which allows controlled cleavage of the polynucleotide strand (e.g., the linker, the primer, or the polynucleotide) by chemical, enzymatic, or photochemical means known in the art and described herein. A scissile site or cleavable site may refer to the linkage of a nucleotide between two other nucleotides in a nucleotide strand (i.e., an internucleosidic linkage). In embodiments, the scissile linkage or cleavable site can be located at any position within the one or more nucleic acid molecules, including at or near a terminal end (e.g., the 3′ end of an oligonucleotide) or in an interior portion of the one or more nucleic acid molecules. In embodiments, conditions suitable for separating a scissile linkage include a modulating the pH and/or the temperature. In embodiments, a scissile site can include at least one acid-labile linkage. For example, an acid-labile linkage may include a phosphoramidate linkage. In embodiments, a phosphoramidate linkage can be hydrolysable under acidic conditions, including mild acidic conditions such as trifluoroacetic acid and a suitable temperature (e.g., 30° C.), or other conditions known in the art, for example Matthias Mag, et al Tetrahedron Letters, Volume 33, Issue 48, 1992, 7319-7322. In embodiments, the scissile site can include at least one photolabile internucleosidic linkage (e.g., o-nitrobenzyl linkages, as described in Walker et al, J. Am. Chem. Soc. 1988, 110, 21, 7170-7177), such as o-nitrobenzyloxymethyl or p-nitrobenzyloxymethyl group(s). In embodiments, the scissile site includes at least one uracil nucleobase. In embodiments, a uracil nucleobase can be cleaved with a uracil DNA glycosylase (UDG) or Formamidopyrimidine DNA Glycosylase Fpg. In embodiments, the scissile linkage site includes a sequence-specific nicking site having a nucleotide sequence that is recognized and nicked by a nicking endonuclease enzyme or a uracil DNA glycosylase. In embodiments, a cleavable site can include a diol linker, disulfide linker, photocleavable linker, abasic site, deoxyuracil triphosphate (dUTP), deoxy-8-Oxo-guanine triphosphate (d-8-oxoG), methylated nucleotide, ribonucleotide, or a sequence containing a modified or unmodified nucleotide that is specifically recognized by a cleaving agent.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site blast.ncbi.nlm.nih.gov/Blast.cgi or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

As used herein, the term “removable” group, e.g., a label or a blocking group or protecting group, is used in accordance with its plain and ordinary meaning and refers to a chemical group that can be removed from a nucleotide analogue such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a nucleotide or nucleotide analogue. In general, the conditions under which a removable group is removed are compatible with a process employing the removable group (e.g., an amplification process or sequencing process).

As used herein, the terms “reversible blocking groups” and “reversible terminators” are used in accordance with their plain and ordinary meanings and refer to a blocking moiety located, for example, at the 3′ position of the nucleotide and may be a chemically cleavable moiety such as an allyl group, an azidomethyl group or a methoxymethyl group, or may be an enzymatically cleavable group such as a phosphate ester. Non-limiting examples of nucleotide blocking moieties are described in applications WO 2004/018497, U.S. Pat. Nos. 7,057,026, 7,541,444, WO 96/07669, U.S. Pat. Nos. 5,763,594, 5,808,045, 5,872,244 and 6,232,465, the contents of which are incorporated herein by reference in their entirety. The nucleotides may be labelled or unlabeled. They may be modified with reversible terminators useful in methods provided herein and may be 3′-O-blocked reversible or 3′-unblocked reversible terminators. In nucleotides with 3′-O-blocked reversible terminators, the blocking group —OR [reversible terminating (capping) group] is linked to the oxygen atom of the 3′-OH of the pentose, while the label is linked to the base, which acts as a reporter and can be cleaved. The 3′-O-blocked reversible terminators are known, and may be, for instance, a 3′-ONH₂ reversible terminator, a 3′-O-allyl reversible terminator, or a 3′-O-azidomethyl reversible terminator. In embodiments, the reversible terminator moiety is attached to the 3′-oxygen of the nucleotide, having the formula:

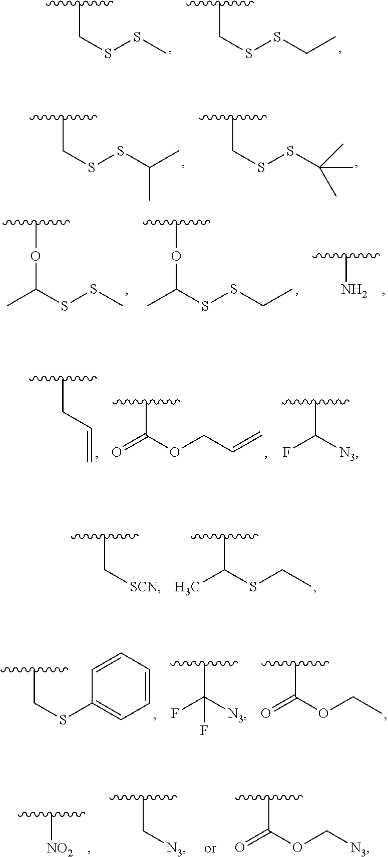

wherein the 3′ oxygen of the nucleotide is not shown in the formulae above. The term “allyl” as described herein refers to an unsubstituted methylene attached to a vinyl group (i.e., —CH═CH₂), having the formula

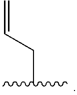 [SMILES: *CC=C]

In embodiments, the reversible terminator moiety is

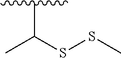 [SMILES: *C(C)SSC]

as described in U.S. Pat. No. 10,738,072, which is incorporated herein by reference for all purposes. For example, a nucleotide including a reversible terminator moiety may be represented by the formula:

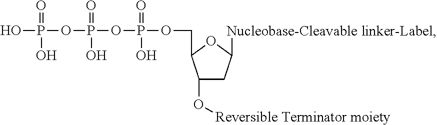

where the nucleobase is adenine or adenine analogue, thymine or thymine analogue, guanine or guanine analogue, or cytosine or cytosine analogue.

In some embodiments, a nucleic acid (e.g., an immobilized oligonucleotide) comprises a molecular identifier or a molecular barcode. As used herein, the term “barcode” or “index” or “unique molecular identifier (UMI)” refers to a known nucleic acid sequence that allows some feature with which the barcode is associated to be identified. Typically, a barcode is unique to a particular feature in a pool of barcodes that differ from one another in sequence, and each of which is associated with a different feature. In embodiments, a barcode is unique in a pool of barcodes that differ from one another in sequence, or is uniquely associated with a particular sample polynucleotide in a pool of sample polynucleotides. In embodiments, every barcode in a pool of adapters is unique, such that sequencing reads comprising the barcode can be identified as originating from a single sample polynucleotide molecule on the basis of the barcode alone. In other embodiments, individual barcode sequences may be used more than once, but adapters comprising the duplicate barcodes are associated with different sequences and/or in different combinations of barcoded adaptors, such that sequence reads may still be uniquely distinguished as originating from a single sample polynucleotide molecule on the basis of a barcode and adjacent sequence information (e.g., sample polynucleotide sequence, and/or one or more adjacent barcodes). In embodiments, barcodes are about or at least about 5, 6, 7, 8, 9, 10, 15, 20, 25, 30, 40, 50, 75 or more nucleotides in length. In embodiments, barcodes are shorter than 20, 15, 10, 9, 8, 7, 6, or 5 nucleotides in length. In embodiments, barcodes are about 10 to about 50 nucleotides in length, such as about 15 to about 40 or about 20 to about 30 nucleotides in length. In a pool of different barcodes, barcodes may have the same or different lengths. In general, barcodes are of sufficient length and comprise sequences that are sufficiently different to allow the identification of associated features (e.g., a binding moiety or analyte) based on barcodes with which they are associated. In embodiments, a barcode can be identified accurately after the mutation, insertion, or deletion of one or more nucleotides in the barcode sequence, such as the mutation, insertion, or deletion of 1, 2, 3, 4, 5, or more nucleotides. In embodiments, each barcode in a plurality of barcodes differs from every other barcode in the plurality by at least three nucleotide positions, such as at least 3, 4, 5, 6, 7, 8, 9, 10, or more nucleotide positions. In some embodiments, substantially degenerate barcodes may be known as random.

In some embodiments, a nucleic acid comprises a label. As used herein, the term “label” or “labels” are used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiment, a nucleotide comprises a label (such as a dye). In embodiments, the label is not associated with any particular nucleotide, but detection of the label identifies whether one or more nucleotides having a known identity were added during an extension step (such as in the case of pyrosequencing).

In embodiments, the detectable label is a fluorescent dye. In embodiments, the detectable label is a fluorescent dye capable of exchanging energy with another fluorescent dye (e.g., fluorescence resonance energy transfer (FRET) chromophores). Examples of detectable agents include imaging agents, including fluorescent and luminescent substances, including, but not limited to, a variety of organic or inorganic small molecules commonly referred to as “dyes,” “labels,” or “indicators.” Examples include fluorescein, rhodamine, acridine dyes, Alexa dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). In embodiments, the detectable moiety is a moiety of a derivative of one of the detectable moieties described immediately above, wherein the derivative differs from one of the detectable moieties immediately above by a modification resulting from the conjugation of the detectable moiety to a compound described herein. The term “cyanine” or “cyanine moiety” as described herein refers to a detectable moiety containing two nitrogen groups separated by a polymethine chain. In embodiments, the cyanine moiety has 3 methine structures (i.e., cyanine 3 or Cy3). In embodiments, the cyanine moiety has 5 methine structures (i.e., cyanine 5 or Cy5). In embodiments, the cyanine moiety has 7 methine structures (i.e., cyanine 7 or Cy7).

As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Exemplary types of polymerases that may be used in the compositions and methods of the present disclosure include the nucleic acid polymerases such as DNA polymerase, DNA- or RNA-dependent RNA polymerase, and reverse transcriptase. In some cases, the DNA polymerase is 9° N polymerase or a variant thereof, E. Coli DNA polymerase I, Bacteriophage T4 DNA polymerase, Sequenase, Taq DNA polymerase, DNA polymerase from Bacillus stearothermophilus, Bst 2.0 DNA polymerase, 9° N polymerase (exo-)A485L/Y409V, Phi29 DNA Polymerase (φ29 DNA Polymerase), T7 DNA polymerase, DNA polymerase II, DNA polymerase III holoenzyme, DNA polymerase IV, DNA polymerase V, VentR DNA polymerase, Therminator™ II DNA Polymerase, Therminator™ III DNA Polymerase, or Therminator™ IX DNA Polymerase. In embodiments, the polymerase is a protein polymerase. Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol υ DNA polymerase, or a thermophilic nucleic acid polymerase (e.g. Therminator γ, 9° N polymerase (exo-), Therminator II, Therminator III, or Therminator IX). In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044). In embodiments, the polymerase is an enzyme described in US 2021/0139884.

As used herein, the term “thermophilic nucleic acid polymerase” refers to a family of DNA polymerases (e.g., 9° N™) and mutants thereof derived from the DNA polymerase originally isolated from the hyperthermophilic archaea, Thermococcus sp. 9 degrees N-7, found in hydrothermal vents at that latitude (East Pacific Rise) (Southworth M W, et al. PNAS. 1996; 93(11):5281-5285). A thermophilic nucleic acid polymerase is a member of the family B DNA polymerases. Site-directed mutagenesis of the 3′-5′ exo motif I (Asp-Ile-Glu or DIE) to AIA, AIE, EIE, EID or DIA yielded polymerase with no detectable 3′ exonuclease activity. Mutation to Asp-Ile-Asp (DID) resulted in reduction of 3′-5′ exonuclease specific activity to <1% of wild type, while maintaining other properties of the polymerase including its high strand displacement activity. The sequence AIA (D141A, E143A) was chosen for reducing exonuclease. Subsequent mutagenesis of key amino acids results in an increased ability of the enzyme to incorporate dideoxynucleotides, ribonucleotides and acyclonucleotides (e.g., Therminator II enzyme from New England Biolabs with D141A/E143A/Y409V/A485L mutations); 3′-amino-dNTPs, 3′-azido-dNTPs and other 3′-modified nucleotides (e.g., NEB Therminator III DNA Polymerase with D141A/E143A/L408S/Y409A/P410V mutations, NEB Therminator IX DNA polymerase), or γ-phosphate labeled nucleotides (e.g., Therminator γ: D141A/E143A/W355A/L408W/R460A/Q461S/K464E/D480V/R484W/A485L). Typically, these enzymes do not have 5′-3′ exonuclease activity. Additional information about thermophilic nucleic acid polymerases may be found in (Southworth M W, et al. PNAS. 1996; 93(11):5281-5285; Bergen K, et al. ChemBioChem. 2013; 14(9):1058-1062; Kumar S, et al. Scientific Reports. 2012; 2:684; Fuller C W, et al. 2016; 113(19):5233-5238; Guo J, et al. Proceedings of the National Academy of Sciences of the United States of America. 2008; 105(27):9145-9150), which are incorporated herein in their entirety for all purposes.

As used herein, the term “exonuclease activity” is used in accordance with its ordinary meaning in the art, and refers to the removal of a nucleotide from a nucleic acid by an enzyme (e.g. DNA polymerase, a lambda exonuclease, Exo I, Exo III, T5, Exo V, Exo VII or the like). For example, during polymerization, nucleotides are added to the 3′ end of the primer strand. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. In embodiments, exonuclease activity may be referred to as “proofreading.” When referring to 3′-5′ exonuclease activity, it is understood that the DNA polymerase facilitates a hydrolyzing reaction that breaks phosphodiester bonds at the 3′ end of a polynucleotide chain to excise the nucleotide. In embodiments, 3′-5′ exonuclease activity refers to the successive removal of nucleotides in single-stranded DNA in a 3′→5′ direction, releasing deoxyribonucleoside 5′-monophosphates one after another. Methods for quantifying exonuclease activity are known in the art, see for example Southworth et al, PNAS Vol 93, 8281-8285 (1996).

As used herein, the term “incorporating” or “chemically incorporating,” when used in reference to a primer and cognate nucleotide, refers to the process of joining the cognate nucleotide to the primer or extension product thereof by formation of a phosphodiester bond. In embodiments, incorporating a nucleotide is catalyzed by an enzyme (e.g., a polymerase).

As used herein, the term “selective” or “selectivity” or the like of a compound refers to the compound's ability to discriminate between molecular targets. For example, a chemical reagent may selectively modify one nucleotide type in that it reacts with one nucleotide type (e.g., cytosines) and not other nucleotide types (e.g., adenine, thymine, or guanine). When used in the context of sequencing, such as in “selectively sequencing,” this term refers to sequencing one or more target polynucleotides from an original starting population of polynucleotides, and not sequencing non-target polynucleotides from the starting population. Typically, selectively sequencing one or more target polynucleotides involves differentially manipulating the target polynucleotides based on known sequence. For example, target polynucleotides may be hybridized to a probe oligonucleotide that may be labeled (such as with a member of a binding pair) or bound to a surface. In embodiments, hybridizing a target polynucleotide to a probe oligonucleotide includes the step of displacing one strand of a double-stranded nucleic acid. Probe-hybridized target polynucleotides may then be separated from non-hybridized polynucleotides, such as by removing probe-bound polynucleotides from the starting population or by washing away polynucleotides that are not bound to a probe. The result is a selected subset of the starting population of polynucleotides, which is then subjected to sequencing, thereby selectively sequencing the one or more target polynucleotides.

As used herein, the terms “specific”, “specifically”, “specificity”, or the like of a compound refers to the agent's ability to cause a particular action, such as binding, to a particular molecular target with minimal or no action to other proteins in the cell.

As used herein, the terms “bind” and “bound” are used in accordance with their plain and ordinary meanings and refer to an association between atoms or molecules. The association can be direct or indirect. For example, bound atoms or molecules may be directly bound to one another, e.g., by a covalent bond or non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). As a further example, two molecules may be bound indirectly to one another by way of direct binding to one or more intermediate molecules, thereby forming a complex.

As used herein, the term “rolling circle amplification (RCA)” refers to a nucleic acid amplification reaction that amplifies a circular nucleic acid template (e.g., single-stranded DNA circles) via a rolling circle mechanism. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers comprising tandem repeat units of the circular nucleic acid template sequence. The rolling circle amplification may be a linear RCA (LRCA), exhibiting linear amplification kinetics (e.g., RCA using a single specific primer), or may be an exponential RCA (ERCA) exhibiting exponential amplification kinetics. Rolling circle amplification may also be performed using multiple primers (multiply primed rolling circle amplification or MPRCA) leading to hyper-branched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in-vitro under isothermal conditions using a suitable nucleic acid polymerase such as Phi29 DNA polymerase. RCA may be performed by using any of the DNA polymerases that are known in the art (e.g., a Phi29 DNA polymerase, a Bst DNA polymerase, or SD polymerase).

As used herein, the term “recombinase polymerase amplification (RPA)” refers to a nucleic acid amplification reaction where recombinase proteins that interact with primers present in a sample mixture to create a recombinase primer complex that reads target DNA and binds accordingly. The recombinase primer complex separates the hydrogen bonds between the two strands of nucleotides of the DNA and replaces them with the complementary regions of the recombinase primer complex, allowing amplification without using fluctuating temperatures to displace adjacent strands.

As used herein, the term “helicase dependent amplification (HDA)” refers to a nucleic acid amplification reaction that does not require thermocycling as a DNA helicase generates single-stranded templates for primer hybridization and subsequent primer extension is done by a DNA polymerase.

As used herein, the term “template walking amplification” refers to an isothermal amplification process based on a template walking mechanism and utilizes low-melting temperature solid-surface homopolymer primers and solution phase primer. In template walking amplification, hybridization of a primer to a template strand is followed by primer extension to form a first extended strand, partial or incomplete denaturation of the extended strand from the template strand. Primer extension in subsequence amplification cycles then involve displacement of first extended strand from the template strand.

As used herein, the term “thermal bridge polymerase chain reaction amplification” refers to a nucleic acid amplification reaction that includes thermally cycling between high temperatures (e.g., 85° C.-95° C.) and low temperatures (e.g., 60° C.-70° C.). Thermal bridge polymerase chain reactions may also include a denaturant, typically at a much lower concentration than traditional chemical bridge polymerase chain reactions.

As used herein, the term “chemical bridge polymerase chain reaction amplification” refers to a nucleic acid amplification reaction that fluidically cycling a denaturant (e.g., formamide) and maintaining the temperature within a narrow temperature range (e.g., +/−5° C.).

As used herein, the term “chemical-thermal bridge polymerase chain reaction amplification” refers to a nucleic acid amplification reaction that combines thermal cycling and chemical denaturants to facilitate optimal strand denaturation and annealing. In embodiments, chemical denaturants are used at significantly lower concentrations than traditional chemical bridge polymerase chain reactions.

As used herein, the terms “sequencing”, “sequence determination”, “determining a nucleotide sequence”, and the like include determination of a partial or complete sequence information, including the identification, ordering, or locations of the nucleotides that comprise the polynucleotide being sequenced, and inclusive of the physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein includes contacting a template and an annealed primer with a suitable polymerase under conditions suitable for polymerase extension and/or sequencing. The sequencing methods are preferably carried out with the target polynucleotide arrayed on a solid substrate. Multiple target polynucleotides can be immobilized on the solid support through linker molecules, or can be attached to particles, e.g., microspheres, which can also be attached to a solid substrate. In embodiments, the solid substrate is in the form of a chip, a bead, a well, a capillary tube, a slide, a wafer, a filter, a fiber, a porous media, or a column. In embodiments, the solid substrate is gold, quartz, silica, plastic, glass, diamond, silver, metal, or polypropylene. In embodiments, the solid substrate is porous.

As used herein, the term “sequencing cycle” is used in accordance with its plain and ordinary meaning and refers to incorporating one or more nucleotides (e.g., nucleotide analogues) to the 3′ end of a polynucleotide with a polymerase, and detecting one or more labels that identify the one or more nucleotides incorporated. In embodiments, one nucleotide (e.g., a modified nucleotide) is incorporated per sequencing cycle. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes, and other substances can be removed between steps by washing. Cycles may include repeating these steps, and the sequence of each cluster is read over the multiple repetitions.

As used herein, the term “sequencing reaction mixture” is used in accordance with its plain and ordinary meaning and refers to an aqueous mixture that contains the reagents necessary to allow a nucleotide or nucleotide analogue to be added to a DNA strand by a DNA polymerase. In embodiments, the sequencing reaction mixture includes a buffer. In embodiments, the buffer includes an acetate buffer, 3-(N-morpholino)propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), and/or salts (e.g., ammonium sulfate, magnesium chloride, sodium chloride, or potassium chloride). As used herein, the term “invasion-reaction mixture” is used in accordance with its plain and ordinary meaning and refers to an aqueous mixture that contains the reagents sufficient to allow a nucleotide or nucleotide analogue to be added to a DNA strand by a DNA polymerase that extends the invasion primer.

As used herein, the term “extending”, “extension” or “elongation” is used in accordance with their plain and ordinary meanings and refer to synthesis by a polymerase of a new polynucleotide strand (e.g., an “extension strand”) complementary to a template strand by adding free nucleotides (e.g., dNTPs) from a reaction mixture that are complementary to the template in a 5′-to-3′ direction, including condensing a 5′-phosphate group of a dNTPs with a 3′-hydroxy group at the end of the nascent (elongating) DNA strand.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 10, 20, 30, 40, 50, 60, 70, 80, 90, 100, 150, 200, 250, or more nucleotide bases. In embodiments, a sequencing read includes reading a barcode and a template nucleotide sequence. In embodiments, a sequencing read includes reading a template nucleotide sequence. In embodiments, a sequencing read includes reading a barcode and not a template nucleotide sequence. In embodiments, a sequencing read is about 25 nucleotide bases. In embodiments, a sequencing read is about 35 nucleotide bases. In embodiments, a sequencing read is about 45 nucleotide bases. In embodiments, a sequencing read is about 55 nucleotide bases. In embodiments, a sequencing read is about 65 nucleotide bases. In embodiments, a sequencing read is about 75 nucleotide bases. In embodiments, a sequencing read is about 85 nucleotide bases. In embodiments, a sequencing read is a string of characters representing the sequence of nucleotides. In embodiments, the length of a sequencing read corresponds to the length of the target sequence. In embodiments, the length of a sequencing read corresponds to the number of sequencing cycles. A sequencing read may be subjected to initial processing (often termed “pre-processing”) prior to annotation. Pre-processing includes filtering out low-quality sequences, sequence trimming to remove continuous low-quality nucleotides, merging paired-end sequences, or identifying and filtering out PCR repeats using known techniques in the art. The sequenced reads may then be assembled and aligned using bioinformatic algorithms known in the art. A sequencing read may be aligned to a reference sequence. In embodiments, a sequencing read includes a computationally derived string corresponding to the detected complementary nucleotide (e.g., a labeled nucleotide). The sequence reads are optionally stored in an appropriate data structure for further evaluation. In embodiments, a first sequencing reaction can generate a first sequencing read. The first sequencing read can provide the sequence of a first region of the polynucleotide fragment. In some embodiments, the nucleic acid template is optionally subjected to one or more additional rounds of sequencing using additional sequencing primers, thereby generating additional sequencing reads.

The term “multiplexing” as used herein refers to an analytical method in which the presence and/or amount of multiple targets, e.g., multiple nucleic acid target sequences, can be assayed simultaneously by using the methods and devices as described herein, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

Complementary single stranded nucleic acids and/or substantially complementary single stranded nucleic acids can hybridize to each other under hybridization conditions, thereby forming a nucleic acid that is partially or fully double stranded. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often include nucleic acid sequences that are substantially complementary to each other.

As used herein, the term “hybridize” or “specifically hybridize” refers to a process where two complementary nucleic acid strands anneal to each other under appropriately stringent conditions. Hybridizations are typically and preferably conducted with oligonucleotides. The terms “annealing” and “hybridization” are used interchangeably to mean the formation of a stable duplex. The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). Those skilled in the art understand how to estimate and adjust the stringency of hybridization conditions such that sequences having at least a desired level of complementarity will stably hybridize, while those having lower complementarity will not. As used herein, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can be further altered by the addition or removal of components of the buffered solution.

As used herein, the term “stringent condition” refers to condition(s) under which a polynucleotide probe or primer will hybridize preferentially to its target sequence, and to a lesser extent to, or not at all to, other sequences. As used herein, “specifically hybridizes” refers to preferential hybridization under hybridization conditions where two nucleic acids, or portions thereof, that are substantially complementary, hybridize to each other and not to other nucleic acids that are not substantially complementary to either of the two nucleic acids. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which includes a double-stranded portion of nucleic acid.

A nucleic acid can be amplified by a suitable method. The term “amplification,” “amplified” or “amplifying” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof (which may be referred to herein as an “amplification product” or “amplification products”). In some embodiments an amplification reaction includes a suitable thermal stable polymerase. Thermal stable polymerases are known and are stable for prolonged periods of time, at temperature greater than 80° C. when compared to common polymerases found in most mammals. In certain embodiments the term “amplification,” “amplified” or “amplifying” refers to a method that includes a polymerase chain reaction (PCR). Conditions conducive to amplification (i.e., amplification conditions) are known and often include at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

As used herein, bridge-PCR (bPCR) amplification is a method for solid-phase amplification as exemplified by the disclosures of U.S. Pat. Nos. 5,641,658; 7,115,400; and U.S. Patent Publ. No. 2008/0009420, each of which is incorporated herein by reference in its entirety. Bridge-PCR involves repeated polymerase chain reaction cycles, cycling between denaturation, annealing, and extension conditions and enables controlled, spatially-localized, amplification, to generate amplification products (e.g., amplicons) immobilized on a solid support in order to form arrays included of colonies (or “clusters”) of immobilized nucleic acid molecule.

A nucleic acid can be amplified by a thermocycling method or by an isothermal amplification method. In some embodiments, a rolling circle amplification method is used. In some embodiments, amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments solid phase amplification includes a nucleic acid amplification reaction including only one species of oligonucleotide primer (e.g., an amplification primer) immobilized to a surface or substrate. In certain embodiments solid phase amplification includes a plurality of different immobilized oligonucleotide primer species. In some embodiments solid phase amplification may include a nucleic acid amplification reaction including one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge amplification, emulsion PCR, WildFire amplification (e.g., US patent publication US2013/0012399), the like or combinations thereof.

Provided herein are methods and compositions for analyzing a sample (e.g., sequencing nucleic acids within a sample). A sample (e.g., a sample including nucleic acid) can be obtained from a suitable subject. A sample can be isolated or obtained directly from a subject or part thereof. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. A fluid or tissue sample from which nucleic acid is extracted may be acellular (e.g., cell-free). Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid).

In some embodiments, a sample includes nucleic acid, or fragments thereof. A sample can include nucleic acids obtained from one or more subjects. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof. A sample may include synthetic nucleic acid.

A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a human subject. A subject can be a patient (e.g., a human patient). In some embodiments a subject is suspected of having a genetic variation or a disease or condition associated with a genetic variation.

The methods and kits of the present disclosure may be applied, mutatis mutandis, to the sequencing of RNA, or to determining the identity of a ribonucleotide.

The terms “bioconjugate group,” “bioconjugate reactive moiety,” and “bioconjugate reactive group” refer to a chemical moiety which participates in a reaction to form a bioconjugate linker (e.g., covalent linker). Non-limiting examples of bioconjugate groups include —NH₂, —COOH, —COOCH₃, —N-hydroxysuccinimide, —N₃, -dibenzylcyclooctyne (DBCO), alkyne, -maleimide,

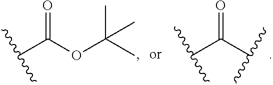

In embodiments, the bioconjugate reactive group may be protected (e.g., with a protecting group). In embodiments, the bioconjugate reactive moiety is

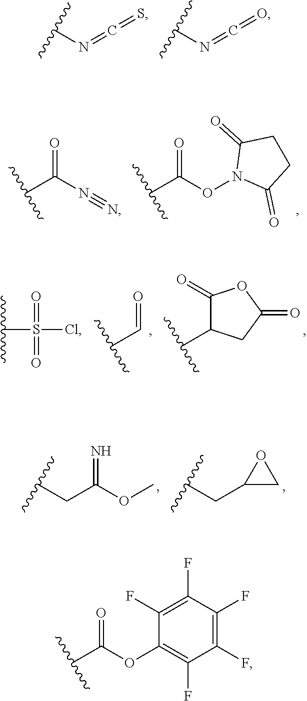

or —NH₂. Additional examples of bioconjugate reactive groups and the resulting bioconjugate reactive linkers may be found in the Bioconjugate Table below:

| Bioconjugate reactive | Bioconjugate reactive | |
|---|---|---|
| group 1 (e.g., | group 2 (e.g., | |
| electrophilic | nucleophilic | |
| bioconjugate | bioconjugate | Resulting Bioconjugate |
| reactive moiety) | reactive moiety) | reactive linker |
| activated esters | amines/anilines | carboxamides |
| acrylamides | thiols | thioethers |
| acyl azides | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carbodiimides | carboxylic acids | N-acylureas or |
| | | anhydrides |
| diazoalkanes | carboxylic acids | esters |
| epoxides | thiols | thioethers |
| haloacetamides | thiols | thioethers |
| haloplatinate | amino | platinum complex |
| haloplatinate | heterocycle | platinum complex |
| haloplatinate | thiol | platinum complex |
| halotriazines | amines/anilines | aminotriazines |
| halotriazines | alcohols/phenols | triazinyl ethers |
| halotriazines | thiols | triazinyl thioethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |

As used herein, the term “bioconjugate” or “bioconjugate linker” refers to the resulting association between atoms or molecules of bioconjugate reactive groups. The association can be direct or indirect. For example, a conjugate between a first bioconjugate reactive group (e.g.,
“\*MERGEFORMAT\*MERGEFORMAT —NH₂, —COOH, —N—
   hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine). In embodiments, the first bioconjugate reactive group (e.g., —COOH) is covalently attached to the second bioconjugate reactive group (e.g.,

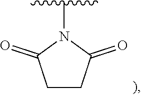 [SMILES: *N1C(=O)CCC1=O]

thereby forming a bioconjugate (e.g.,

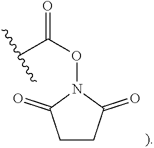 [SMILES: *C(=O)ON1C(=O)CCC1=O]

In embodiments, the first bioconjugate reactive group (e.g., —NH₂) is covalently attached to the second bioconjugate reactive group (e.g.,

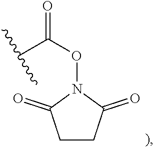 [SMILES: *C(=O)ON1C(=O)CCC1=O]

thereby forming a bioconjugate (e.g.,

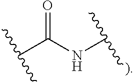 [SMILES: *NC(*)=O]

In embodiments, the first bioconjugate reactive group (e.g., a coupling reagent) is covalently attached to the second bioconjugate reactive group (e.g.,

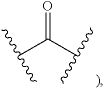 [SMILES: *C(*)=O]

thereby forming a bioconjugate (e.g.,

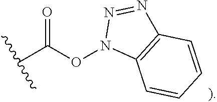 [SMILES: *C(=O)On1nnc2ccccc21]

In embodiments, the first bioconjugate reactive group (e.g., azide moiety) is covalently attached to the second bioconjugate reactive group (e.g., an alkyne moiety) to form a 5-membered heteroatom ring. In embodiments, the first bioconjugate reactive group (e.g., azide moiety) is covalently attached to the second bioconjugate reactive group (e.g., an DBCO moiety) to form a bioconjugate linker.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate includes a molecular entity derived from the reaction of an unsaturated bond, such as a maleimide, and a sulfhydryl group.

Useful bioconjugate reactive groups used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; (o) biotin conjugate can react with avidin or streptavidin to form a avidin-biotin complex or streptavidin-biotin complex.

The term “covalent linker” is used in accordance with its ordinary meaning and refers to a divalent moiety which connects at least two moieties to form a molecule.

The term “non-covalent linker” is used in accordance with its ordinary meaning and refers to a divalent moiety which includes at least two molecules that are not covalently linked to each other but are capable of interacting with each other via a non-covalent bond (e.g., electrostatic interactions (e.g., ionic bond, hydrogen bond, halogen bond) or van der Waals interactions (e.g., dipole-dipole, dipole-induced dipole, London dispersion). In embodiments, the non-covalent linker is the result of two molecules that are not covalently linked to each other that interact with each other via a non-covalent bond.

The term “adapter” as used herein refers to any oligonucleotide that can be ligated to a nucleic acid molecule, thereby generating nucleic acid products that can be sequenced on a sequencing platform (e.g., an Illumina or Singular Genomics™ sequencing platform). In embodiments, adapters include two reverse complementary oligonucleotides forming a double-stranded structure. In embodiments, an adapter includes two oligonucleotides that are complementary at one portion and mismatched at another portion, forming a Y-shaped or fork-shaped adapter that is double stranded at the complementary portion and has two overhangs at the mismatched portion. Since Y-shaped adapters have a complementary, double-stranded region, they can be considered a special form of double-stranded adapters. When this disclosure contrasts Y-shaped adapters and double stranded adapters, the term “double-stranded adapter” or “blunt-ended” is used to refer to an adapter having two strands that are fully complementary, substantially (e.g., more than 90% or 95%) complementary, or partially complementary. In embodiments, adapters include sequences that bind to sequencing primers. In embodiments, adapters include sequences that bind to immobilized oligonucleotides (e.g., P7 and P5 sequences, or S1 and S2 sequences) or reverse complements thereof. In embodiments, the adapter is substantially non-complementary to the 3′ end or the 5′ end of any target polynucleotide present in the sample. In embodiments, the adapter can include a sequence that is substantially identical, or substantially complementary, to at least a portion of a primer, for example a universal primer. In embodiments, the adapter can include an index sequence (also referred to as barcode or tag) to assist with downstream error correction, identification or sequencing. In embodiments, greater than four types of adapters are contemplated herein, for example 5, 6, 7, 8, 9, 10, 11, or 12 adapters.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed. The upper and lower limits of any such smaller range (within a more broadly recited range) may independently be included in the smaller ranges, or as particular values themselves, and are also encompassed, subject to any specifically excluded limit in the stated range. Where the stated range includes one or both of the limits, ranges excluding either or both of those included limits are also included.

“Synthetic” agents refer to non-naturally occurring agents, such as enzymes or nucleotides.

As used herein, the term “feature” refers a site (i.e., a physical location) on a solid support for one or more molecule(s). A feature can contain only a single molecule or it can contain a population of several molecules of the same species (i.e., a cluster). Features of an array are typically discrete. The discrete features can be contiguous, or they can have spaces between each other. An “optically resolvable feature” refers to a feature capable of being distinguished from other features. Optics and sensor resolution has a finite limit as to a resolvable area. The Rayleigh criterion for the diffraction limit to resolution states that two images are just resolvable when the center of the diffraction pattern of one object is directly over the first minimum of the diffraction pattern of the other object. The minimal distance between two resolvable objects, r, is proportional to the wavelength of light and inversely proportional to the numerical aperture (NA). That is, the minimal distance between two resolvable objects is provided as r=0.61 wavelength/NA. If detecting light in the UV-vis spectrum (about 100 nm to about 900 nm), the remaining mutable variable to increase the resolution is the NA of the objective lens. A lens with a large NA will be able to resolve finer details. For example, a lens with larger NA is capable of detecting more light and so it produces a brighter image. Thus, a large NA lens provides more information to form a clear image, and so its resolving power will be higher. Typical dry objectives have an NA of about 0.80 to about 0.95. Higher NAs may be obtained by increasing the imaging medium refractive index between the object and the objective front lens for example immersing the lens in water (refractive index=1.33), glycerin (refractive index=1.47), or immersion oil (refractive index=1.51). Most oil immersion objectives have a maximum numerical aperture of 1.4, with the typical objectives having an NA ranging from 1.0 to 1.35.

It will be understood that the steps of the methods set forth herein can be carried out in a manner to expose an entire site or a plurality of sites of an array with the treatment. For example, a step that involves extension of a primer can be carried out by delivering primer extension reagents to an array such that multiple nucleic acids (e.g., different nucleic acids in a mixture) at each of one or more sites of the array are contacted with the primer extension reagents. Similarly, a step of deblocking a blocked primer extension product can be carried out by exposing an array with a deblocking treatment such that multiple nucleic acids (e.g. different nucleic acids in a mixture) at each of one or more sites of the array are contacted with the treatment.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### II. Compositions & Kits

In an aspect is provided a solid support including a plurality of amplification sites (e.g., features or wells of a multiwell container), wherein each amplification site includes a population of first platform primers, a population of second platform primers, and a population of third platform primers, wherein each of the third platform primers include a cleavable site. In embodiments, each of the populations have a different platform primer binding sequence relative to each population. In embodiments, each of the different populations have a common platform primer binding sequence within each population. In embodiments, each of the platform primers include a sequencing primer binding sequence. In embodiments, the first population of platform primers include the same sequencing primer binding sequence as the third population of platform primers. In embodiments, the second population of platform primers include the same sequencing primer binding sequence as the third population of platform primers. In embodiments, each amplification site is a cluster on a surface of a substrate that includes multiple platform primers selected from a population of first platform primers, a population of second platform primers and a population of third platform primers, wherein each of the third platform primers include a cleavable site. In embodiments, each platform primer within an amplification site is immobilized onto the solid support. In embodiments, the population of first platform primers, population of second platform primers, and a population of third platform primers within an amplification site are all immobilized. In embodiments, each platform primer of the population of first platform primers is complementary to a first platform primer binding sequence of a first oligonucleotide. In embodiments, each platform primer of the population of second platform primers is complementary to a second platform primer binding sequence of a second oligonucleotide. In embodiments, each platform primer of the population of third platform primers is complementary to a third platform primer binding sequence of a third oligonucleotide. In embodiments, the population of first platform primers, the population of second platform primers, and the population of third platform primers are not substantially complementary.

In embodiments, each of platform primers (e.g., immobilized platform primers) is about 12 to about 50 nucleotides in length. In embodiments, each of the platform primers (e.g., immobilized platform primers) is about 5 to about 25 nucleotides in length. In embodiments, each of the platform primers (e.g., immobilized platform primers) is about 10 to about 40 nucleotides in length. In embodiments, each of the platform primers (e.g., immobilized platform primers) is about 5 to about 100 nucleotides in length. In embodiments, each of the platform primers (e.g., immobilized platform primers) is about 20 to 200 nucleotides in length. In embodiments, each of the platform primers (e.g., immobilized platform primers) about or at least about 5, 6, 7, 8, 9, 10, 12, 15, 18, 20, 25, 30, 35, 40, 50 or more nucleotides in length.

In embodiments, the platform primer includes a sequence selected from SEQ ID NO:5, SEQ ID NO:9, SEQ ID NO:85, SEQ ID NO:92, SEQ ID NO:90, SEQ ID NO:88, SEQ ID NO:117, SEQ ID NO:119, SEQ ID NO:121, or SEQ ID NO:123. In embodiments, the platform primer includes a sequence selected from SEQ ID NO:7, SEQ ID NO:30, SEQ ID NO:87, SEQ ID NO:89, SEQ ID NO:91, SEQ ID NO:86, SEQ ID NO:118, SEQ ID NO:120, SEQ ID NO:122, or SEQ ID NO:124. In embodiments, each oligonucleotide includes the sequence of SEQ ID NO:2, SEQ ID NO:28, SEQ ID NO:109, SEQ ID NO:111, SEQ ID NO:113, SEQ ID NO:115, SEQ ID NO:141, SEQ ID NO:143, SEQ ID NO: 145, SEQ ID NO:147, or a sequence greater than 90% homologous thereto. In embodiments, each oligonucleotide includes SEQ ID NO:2. In embodiments, each oligonucleotide includes SEQ ID NO:28. In embodiments, each oligonucleotide includes SEQ ID NO:109. In embodiments, each oligonucleotide includes SEQ ID NO:111. In embodiments, each oligonucleotide includes SEQ ID NO:113. In embodiments, each oligonucleotide includes SEQ ID NO:115. In embodiments, each oligonucleotide includes SEQ ID NO:141. In embodiments, each oligonucleotide includes SEQ ID NO:143. In embodiments, each oligonucleotide includes SEQ ID NO: 145. In embodiments, each oligonucleotide includes SEQ ID NO:147. Exemplary hybridization conditions may include hybridization in a buffer of 40% formamide, 1 M NaCl, 1% SDS at 37° C., and a wash in 1×SSC at 45° C. In embodiments, capable of hybridizing includes hybridization at 5×SSC and 40° C. In embodiments, hybridization occurs when the two oligonucleotides are 100% complementary. In embodiments, hybridization occurs when the two oligonucleotides are greater than 99% complementary. In embodiments, hybridization occurs when the two oligonucleotides are greater than 98% complementary. In embodiments, hybridization occurs when the two oligonucleotides are 99% complementary. In embodiments, hybridization occurs when the two oligonucleotides are 98% complementary. In embodiments, capable of hybridizing includes hybridization in a buffer including 20-200 mM KCl or NaCl, 0.5-12 mM Mg²⁺, about 1-3M betaine, and about 0-10% DMSO.

In embodiments, each oligonucleotide is capable of hybridizing (e.g., via specific hybridization) to SEQ ID NO:7, SEQ ID NO:30, SEQ ID NO:87, SEQ ID NO:89, SEQ ID NO:91, SEQ ID NO:86, SEQ ID NO:118, SEQ ID NO:120, SEQ ID NO:122, or SEQ ID NO:124. In embodiments, each oligonucleotide is capable of specifically hybridizing to SEQ ID NO:7, SEQ ID NO:30, SEQ ID NO:87, SEQ ID NO:89, SEQ ID NO:91, SEQ ID NO:86, SEQ ID NO:118, SEQ ID NO: 120, SEQ ID NO:122, or SEQ ID NO:124.

In embodiments, capable of hybridizing includes hybridization at 5×SSC and 40° C. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can be further altered by the addition or removal of components of the buffered solution. In embodiments, hybridization may occur in a hybridization solution which can include any combination of 50% formamide, 5×SSC (0.75 M NaCl, 0.075 M sodium citrate), 50 mM sodium phosphate (pH 6.8), 0.1% sodium pyrophosphate, 5×Denhardt's solution, 0.1% SDS, and/or 10% dextran sulfate. Exemplary hybridization conditions may include hybridization in a buffer of 40% formamide, 1 M NaCl, 1% SDS at 37° C., and a wash in 1×SSC at 45° C. In embodiments, capable of hybridizing includes hybridization at 5×SSC and 40° C. In embodiments, hybridization occurs when the two oligonucleotides are 100% complementary. In embodiments, hybridization occurs when the two oligonucleotides are greater than 99% complementary. In embodiments, hybridization occurs when the two oligonucleotides are greater than 98% complementary. In embodiments, hybridization occurs when the two oligonucleotides are 99% complementary. In embodiments, hybridization occurs when the two oligonucleotides are 98% complementary. In embodiments, capable of hybridizing includes hybridization in a buffer including 20-200 mM KCl or NaCl, 0.5-12 mM Mg²⁺, about 1-3M betaine, and about 0-10% DMSO.

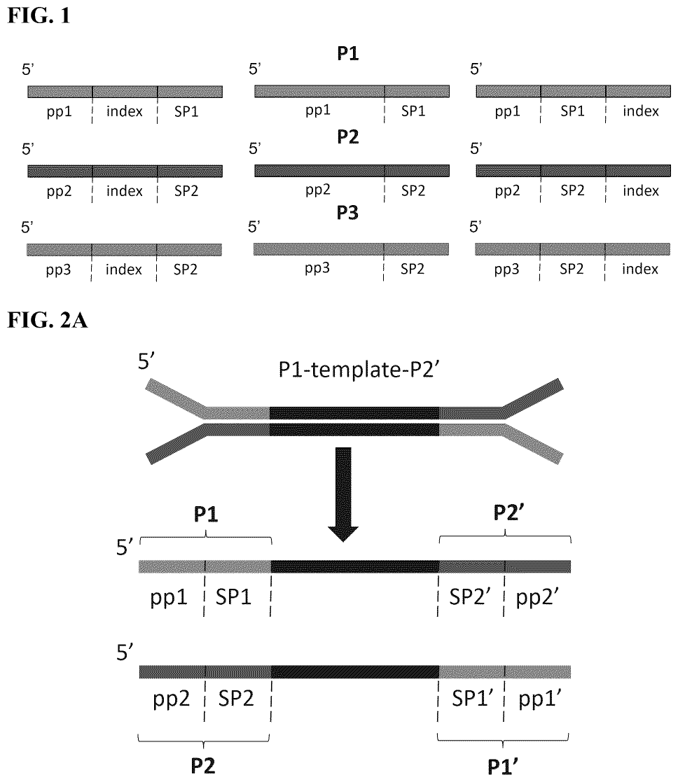

In reference to a “first end” and/or a “second end” of a nucleic acid molecule, it is understood that the “end” is in reference to the sequence of nucleotides at or near the terminus of the molecule. The first end and/or the second end may include nucleotides at the immediate 3′ and/or 5′, and thus the first end if on the 5′ portion of the nucleic acid molecule may include a terminal nucleotide, which includes a 5′ phosphate group attached to the fifth carbon in the sugar-ring of the deoxyribose sugar ring. Alternatively, if the first end (or second end) is on the 3′ portion of the nucleic acid molecule, the first end may include a terminal hydroxyl (—OH) chemical group attached to the third carbon in the sugar ring. As illustrated in FIG. 1, the first end may include all or a portion the pp1 sequence and/or all or a portion of the SP1 sequence. In embodiments, the first end includes a portion of the full pp1 sequence, or a complement thereof. Similarly, in embodiments, the second end includes a portion of the pp2 sequence, or a complement thereof. In embodiments, the first end is the 5′ end and the second end is the 3′ end. In embodiments, the first end includes a 5′ phosphate moiety. In embodiments, the second end includes a 3′-OH (i.e., a 3′-hydroxyl) moiety. In embodiments, the first end and/or the second end includes the sequence as provided herein, in addition to one or more spacer nucleotides.

In some embodiments, each of the platform primers is an oligonucleotide moiety is capable of hybridizing to a complementary sequence of polynucleotide containing a platform binding sequence, a template sequence, and a second platform primer sequence (i.e., an oligonucleotide). In embodiments, the oligonucleotide moiety includes DNA. In embodiments, the oligonucleotide moiety includes RNA. In embodiments, the oligonucleotide moiety is DNA. In embodiments, the oligonucleotide moiety is RNA. In embodiments, the oligonucleotide moiety includes a single-stranded DNA. In embodiments, the oligonucleotide moiety includes a single-stranded RNA. In embodiments, the oligonucleotide moiety is a single-stranded DNA. In embodiments, the oligonucleotide moiety is a single-stranded RNA. In embodiments, the oligonucleotide moiety is a nucleic acid sequence complementary to a target polynucleotide (e.g., complementary to a common adapter sequence of the target polynucleotide).

In embodiments, each of the platform primers is an oligonucleotide moiety that includes one or more phosphorothioate nucleotides. In embodiments, each of the platform primers include a plurality of phosphorothioate nucleotides. In embodiments, about or at least about 20%, 30%, 40%, 50%, 60%, 70%, 80%, 90%, or about 100% of the nucleotides in the platform primers are phosphorothioate nucleotides. In embodiments, most of the nucleotides in the platform primers are phosphorothioate nucleotides. In embodiments, all of the nucleotides in the immobilized platform primers are phosphorothioate nucleotides. In embodiments, none of the nucleotides in the immobilized platform primers are phosphorothioate nucleotides. In embodiments, the 5′ end of the immobilized platform primer includes one or more phosphorothioate nucleotides. In embodiments, the 5′ end of the immobilized platform primer includes between one and five phosphorothioate nucleotides.

In embodiments, each of the platform primers of the population of third platform primers includes a cleavable site. The cleavable site in the third platform primer is a site which allows controlled cleavage of the polynucleotide strand by chemical, enzymatic, or photochemical means. In embodiments, the cleavable site includes a diol linker, disulfide linker, photocleavable linker, abasic site, deoxyuracil triphosphate (dUTP), deoxy-8-Oxo-guanine triphosphate (d-8-oxoG), methylated nucleotide, ribonucleotide, or a sequence containing a modified or unmodified nucleotide that is specifically recognized by a cleaving agent. In embodiments, the cleavable site includes one or more deoxyuracil nucleobases (dUs). In embodiments, the cleavable site includes one or more ribonucleotides. In embodiments, the cleavable site includes 2 to 5 ribonucleotides. In embodiments, the cleavable site includes one ribonucleotide. In embodiments, the cleavable site includes more than one ribonucleotide. In embodiments, the cleavable site includes deoxyuracil triphosphate (dUTP) or deoxy-8-oxo-guanine triphosphate (d-8-oxoG). The cleavable site can be cleaved using methods described herein. In embodiments, the first and second platform primers do not include a cleavage site. In embodiments, the first or second platform primers include an orthogonal cleavage site with respect to the third platform primer.

In embodiments, each population of platform primers on the solid support is immobilized to the solid support. In embodiments, each population of platform primers on the solid support is immobilized to a polymer. In embodiments, the solid support includes a first, second and third plurality of platform primers (immobilized oligonucleotides), wherein the immobilized oligonucleotides of each plurality are different (e.g., S1, S2, S3) and the third plurality of immobilized oligonucleotides includes a cleavable site.

In embodiments, there are at least 3 different populations, but can also include more populations, for example 3 or 4 different libraries, of polynucleotides at a single feature (e.g., a discrete area) of a solid support, wherein the feature includes: a first complex including a first population of polynucleotides (i.e. a first platform primer) attached to the solid support, a second complex including a second population of polynucleotides (i.e. a second platform primer) attached to the solid support, and a third complex including a third population of polynucleotides (i.e. a third platform primer attached to the solid support) wherein each of the third platform primers include a cleavable site, and optionally a fourth complex including a fourth population of polynucleotides (i.e. a fourth platform primer sequence) attached to the solid support. In embodiments, the solid support includes a plurality of features. In embodiments, the feature is about 0.2 m to about 2 m in diameter. In embodiments, the feature is about 0.2-1.5 μm in diameter. In some embodiments, the diameter of the feature is less than 700 nm, less than 600 nm, less than 500 nm, less than 400 nm, less than 300 nm, less than 200 nm, or less than 100 nm. It is also understood that the size of the features on the array can be of various sizes and will ultimately depend on the systems and/or apparatus used to analyze later reactions. The wells of a plurality of wells can be spaced at the same distance or at different distances. The spacing of wells can be expressed, e.g., as the interspatial distance between two wells or as the “pitch,” which includes the interspatial distance between two wells and the diameter of one well.

In embodiments, the platform primers are each attached to the solid support (i.e., immobilized on the surface of a solid support). The platform primers (i.e. polynucleotides) can be fixed to surface by a variety of techniques, including covalent attachment and non-covalent attachment. In embodiments, the platform primers (e.g. polynucleotides) are confined to an area of a discrete region (referred to as a cluster). The discrete regions may have defined locations in a regular array, which may correspond to a rectilinear pattern, circular pattern, hexagonal pattern, or the like. A regular array of such regions is advantageous for detection and data analysis of signals collected from the arrays during an analysis. These discrete regions are separated by interstitial regions. As used herein, the term “interstitial region” refers to an area in a substrate or on a surface that separates other areas (e.g., clusters) of the substrate or surface. For example, an interstitial region can separate one concave feature of an array from another concave feature of the array. The two regions that are separated from each other can be discrete, lacking contact with each other. In another example, an interstitial region can separate a first portion of a feature from a second portion of a feature. In embodiments the interstitial region is continuous whereas the features are discrete, for example, as is the case for an array of wells in an otherwise continuous surface. The separation provided by an interstitial region can be partial or full separation. Interstitial regions will typically have a surface material that differs from the surface material of the features on the surface. For example, features of an array can have polynucleotides that exceeds the amount or concentration present at the interstitial regions. In some embodiments the polynucleotides and/or primers may not be present at the interstitial regions. In embodiments, at least two different primers are attached to the solid support (e.g., a forward and a reverse primer), which facilitates generating multiple amplification products from the first extension product or a complement thereof.

In embodiments, the platform primers are provided in a clustered array. In embodiments, the clustered array includes a plurality of platform primers localized to discrete sites on a solid support. In embodiments, the solid support is a bead. In embodiments, the solid support is substantially planar. In embodiments, the solid support is contained within a flow cell. Flow cells provide a convenient format for housing an array of clusters produced by the methods described herein, in particular when subjected to an SBS or other detection technique that involves repeated delivery of reagents in cycles. For example, to initiate a first SBS cycle, one or more labeled nucleotides and a DNA polymerase in a buffer, can be flowed into/through a flow cell that houses an array of clusters. The clusters of an array where primer extension causes a labeled nucleotide to be incorporated can then be detected. Optionally, the nucleotides can further include a reversible termination moiety that temporarily halts further primer extension once a nucleotide has been added to a primer. For example, a nucleotide analog having a reversible terminator moiety can be added to a primer such that subsequent extension cannot occur until a deblocking agent (e.g., a reducing agent) is delivered to remove the moiety. Thus, for embodiments that use reversible termination, a deblocking reagent (e.g., a reducing agent) can be delivered to the flow cell (before, during, or after detection occurs). Washes can be carried out between the various delivery steps as needed. The cycle can then be repeated N times to extend the primer by N nucleotides, thereby detecting a sequence of length N. Example SBS procedures, fluidic systems and detection platforms that can be readily adapted for use with an array produced by methods of the present disclosure are described, for example, in Bentley et al., Nature 456:53-59 (2008), US Patent Publication 2018/0274024, WO 2017/205336, US Patent Publication 2018/0258472, each of which are incorporated herein in their entirety for all purposes.

In embodiments, the solid support is selected from a flow cell, bead, chip, capillary, plate, membrane, wafer, comb, pin, nanoparticle, multi-well container, or unpatterned solid support. In embodiments, the solid support is contained within a flow cell. In embodiments, the solid support is a flow cell. In embodiments, the solid support is a bead. In embodiments, the solid support is a nanoparticle. In embodiments, the solid support is substantially planar. In embodiments, the solid support is a multiwell container. In embodiments, the solid support is an unpatterned solid support.

In embodiments, the solid support includes a plurality of wells (e.g., a billion or more wells). In embodiments, the wells (e.g., each well) is separated by about 0.1 μm to about 5.0 μm. In embodiments, the wells (e.g., each well) is separated by about 0.2 μm to about 2.0 μm. In embodiments, the wells (e.g., each well) is separated by about 0.5 μm to about 1.5 μm. In embodiments, the wells of the solid support are all the same size. In embodiments, one or more wells are different sizes (e.g., one population of wells are 1.0 μm in diameter, and a second population are 0.5 μm in diameter). In embodiments, the solid support is a glass slide about 75 mm by about 25 mm. In embodiments, the solid support includes a resist (e.g., a photoresist or nanoimprint resist including a crosslinked polymer matrix attached to the solid support).

In embodiments, the density of wells on the solid support may be tuned. For example, in embodiments, the multiwell container includes a density of at least about 100 wells per mm², about 1,000 wells per mm², about 0.1 million wells per mm², about 1 million wells per mm², about 2 million wells per mm², about 5 million wells per mm², about 10 million wells per mm², about 50 million wells per mm², or more. In embodiments, the multiwell container includes no more than about 50 million wells per mm², about 10 million wells per mm², about 5 million wells per mm², about 2 million wells per mm², about 1 million wells per mm², about 0.1 million wells per mm², about 1,000 wells per mm², about 100 wells per mm², or less. In embodiments, the solid support includes about 500, 1,000, 2,500, 5,000, or about 25,000 wells per mm². In embodiments, the solid support includes about 1×10⁶ to about 1×10¹² wells. In embodiments, the solid support includes about 1×10⁷ to about 1×10¹² wells. In embodiments, the solid support includes about 1×10⁸ to about 1×10¹² wells. In embodiments, the solid support includes about 1×10⁶ to about 1×10⁹ wells. In embodiments, the solid support includes about 1×10⁹ to about 1×10¹⁰ wells. In embodiments, the solid support includes about 1×10⁷ to about 1×10⁹ wells. In embodiments, the solid support includes about 1×10⁸ to about 1×10⁹ wells. In embodiments, the solid support includes about 1×10⁶ to about 1×10⁸ wells. In embodiments, the solid support includes about 1×10⁶, 1×10⁷, 1×10⁸, 1×10⁹, 1×10¹⁰, 1×10¹¹, 1×10¹², 5×10¹², or more wells. In embodiments, the solid support includes about 1.8×10⁹, 3.7×10⁹, 9.4×10⁹, 1.9×10¹⁰, or about 9.4×10¹⁰ wells. In embodiments, the solid support includes about 1×10⁶ or more wells. In embodiments, the solid support includes about 1×10⁷ or more wells. In embodiments, the solid support includes about 1×10⁸ or more wells. In embodiments, the solid support includes about 1×10⁹ or more wells. In embodiments, the solid support includes about 1×10¹⁰ or more wells. In embodiments, the solid support includes about 1×10¹¹ or more wells. In embodiments, the solid support includes about 1×10¹² or more wells. In embodiments, the solid support is a glass slide. In embodiments, the solid support is a about 75 mm by about 25 mm. In embodiments, the solid support includes one, two, three, or four channels.

In embodiments, the features and/or the wells have a mean or median separation from one another of about 0.5-5 μm. In embodiments, the mean or median separation is about 0.1-10 microns, 0.25-5 microns, 0.5-2 microns, 1 micron, or a number or a range between any two of these values. In embodiments, the mean or median separation is about or at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 μm, or a number or a range between any two of these values. In embodiments, the mean or median separation is about or at least about 0.1 μm. In embodiments, the mean or median separation is about or at least about 0.2 μm. In embodiments, the mean or median separation is about or at least about 0.3 μm. In embodiments, the mean or median separation is about or at least about 0.4 μm. In embodiments, the mean or median separation is about or at least about 0.5 μm. In embodiments, the mean or median separation is about or at least about 0.6 μm. In embodiments, the mean or median separation is about or at least about 0.7 μm. In embodiments, the mean or median separation is about or at least about 0.8 μm. In embodiments, the mean or median separation is about or at least about 0.9 μm. In embodiments, the mean or median separation is about or at least about 1.0 μm. In embodiments, the mean or median separation is about or at least about 1.1 μm. In embodiments, the mean or median separation is about or at least about 1.2 μm. In embodiments, the mean or median separation is about or at least about 1.3 μm. In embodiments, the mean or median separation is about or at least about 1.4 μm. In embodiments, the mean or median separation is about or at least about 1.5 μm. In embodiments, the mean or median separation is about or at least about 1.6 μm. In embodiments, the mean or median separation is about or at least about 1.7 μm. In embodiments, the mean or median separation is about or at least about 1.8 μm. In embodiments, the mean or median separation is about or at least about 1.9 μm. In embodiments, the mean or median separation is about or at least about 2.0 μm. In embodiments, the mean or median separation is about or at least about 2.1 μm. In embodiments, the mean or median separation is about or at least about 2.2 μm. In embodiments, the mean or median separation is about or at least about 2.3 μm. In embodiments, the mean or median separation is about or at least about 2.4 μm. In embodiments, the mean or median separation is about or at least about 2.5 μm. In embodiments, the mean or median separation is about or at least about 2.6 μm. In embodiments, the mean or median separation is about or at least about 2.7 μm. In embodiments, the mean or median separation is about or at least about 2.8 μm. In embodiments, the mean or median separation is about or at least about 2.9 μm. In embodiments, the mean or median separation is about or at least about 3.0 μm. In embodiments, the mean or median separation is about or at least about 3.1 μm. In embodiments, the mean or median separation is about or at least about 3.2 μm. In embodiments, the mean or median separation is about or at least about 3.3 μm. In embodiments, the mean or median separation is about or at least about 3.4 μm. In embodiments, the mean or median separation is about or at least about 3.5 μm. In embodiments, the mean or median separation is about or at least about 3.6 μm. In embodiments, the mean or median separation is about or at least about 3.7 μm. In embodiments, the mean or median separation is about or at least about 3.8 μm. In embodiments, the mean or median separation is about or at least about 3.9 μm. In embodiments, the mean or median separation is about or at least about 4.0 μm. In embodiments, the mean or median separation is about or at least about 4.1 μm. In embodiments, the mean or median separation is about or at least about 4.2 μm. In embodiments, the mean or median separation is about or at least about 4.3 μm. In embodiments, the mean or median separation is about or at least about 4.4 μm. In embodiments, the mean or median separation is about or at least about 4.5 μm. In embodiments, the mean or median separation is about or at least about 4.6 μm. In embodiments, the mean or median separation is about or at least about 4.7 μm. In embodiments, the mean or median separation is about or at least about 4.8 μm. In embodiments, the mean or median separation is about or at least about 4.9 μm. In embodiments, the mean or median separation is about or at least about 5.0 μm. The mean or median separation may be measured center-to-center (i.e., the center of one well to the center of a second well). In embodiments of the methods provided herein, the wells have a mean or median separation (measured center-to-center) from one another of about 0.5-5 μm. The mean or median separation may be measured edge-to-edge (i.e., the edge of well to the edge of a second well). In embodiments, the wells have a mean or median separation (measured edge-to-edge) from one another of about 0.2-1.5 μm. In embodiments, the wells have a mean or median separation (measured center-to-center) from one another of about 0.7-1.5 μm.

Neighboring features of an array can be discrete one from the other in that they do not overlap. Accordingly, the features can be adjacent to each other or separated by a gap (e.g., an interstitial space). In embodiments where features are spaced apart, neighboring sites can be separated, for example, by a distance of less than 10 μm, 5 μm, 1 μm, 0.9 μm, 0.8 μm, 0.7 μm, 0.6 μm, 0.5 μm, or less. The layout of features on an array can also be understood in terms of center-to-center distances between neighboring features. An array useful herein can have neighboring features with center-to-center spacing of less than about 10 μm, 5 μm, 1 μm, 0.9 μm, 0.8 μm, 0.7 μm, 0.6 μm, 0.5 μm, 0.4 μm, or less. In embodiments, the array has neighboring features with center-to-center spacing of less than about 10 m. In embodiments, the array has neighboring features with center-to-center spacing of less than about 5 m. In embodiments, the array has neighboring features with center-to-center spacing of less than about 1 μm. In embodiments, the array has neighboring features with center-to-center spacing of less than about 0.9 μm. In embodiments, the array has neighboring features with center-to-center spacing of less than about 0.8 μm. In embodiments, the array has neighboring features with center-to-center spacing of less than about 0.7 μm. In embodiments, the array has neighboring features with center-to-center spacing of less than about 0.6 μm. In embodiments, the array has neighboring features with center-to-center spacing of less than about 0.5 μm. In embodiments, the array has neighboring features with center-to-center spacing of less than about 0.4 μm. Furthermore, it will be understood that the distance values described above and elsewhere herein can represent an average distance between neighboring features of an array. As such, not all neighboring features need to fall in the specified range unless specifically indicated to the contrary, for example, by a specific statement that the distance constitutes a threshold distance between all neighboring features of an array.

In embodiments, the three populations of platform primers are present at a density of about 100 oligonucleotides per μm² to about 1,000,000 oligonucleotides per μm². In embodiments, the three populations of platform primers are present at a density of about 100 oligonucleotides per μm² to about 1,000 oligonucleotides per μm². In embodiments, the three populations of platform primers are present at a density of about 100 oligonucleotides per μm² to about 10,000 oligonucleotides per μm². In embodiments, the plurality of oligonucleotides is present at a density of about 100 oligonucleotides per μm² to about 100,000 oligonucleotides per μm². In embodiments, the three populations of platform primers a represent at a density of about 100 oligonucleotides per μm² to about 500,000 oligonucleotides per μm². In embodiments, the three populations of platform primers are present at a density of about 100, 1,000, 10,000, 50,000, 100,000, 250,000, 500,000, 750,000, or 1,000,000 oligonucleotides per μm².

The arrays and solid supports for some embodiments have at least one surface located within a flow cell. Flow cells provide a convenient format for housing an array of clusters produced by the methods described herein, in particular when subjected to an SBS or other detection technique that involves repeated delivery of reagents in cycles.

In embodiments, the solid support is a multiwell container or an unpatterned solid support (e.g., an unpatterned surface). In embodiments, the solid support is a glass slide including a polymer coating (e.g., a hydrophilic polymer coating). In embodiments, the polymer coating includes a plurality of immobilized oligonucleotides (e.g., the platform primers which are complementary to the platform primer binding sequence of the adapter). In embodiments, the solid support is an unpatterned solid support.

In embodiments, the surface of the solid support includes a glass surface including a polymer coating. In embodiments, the surface is glass or quartz, such as a microscope slide, having a surface that is uniformly silanized. This may be accomplished using conventional protocols, such as those described in Beattie et al (1995), Molecular Biotechnology, 4: 213. Such a surface is readily treated to permit end-attachment of oligonucleotides (e.g., forward and reverse primers) prior to amplification. In embodiments the surface further includes a polymer coating, which contains functional groups capable of immobilizing primers. In some embodiments, the surface includes a patterned surface suitable for immobilization of primers in an ordered pattern. A patterned surface refers to an arrangement of different regions in or on an exposed layer of a substrate. For example, one or more of the regions can be features (e.g., clusters) where one or more primers are present. The features can be separated by interstitial regions where capture primers are not present. In some embodiments, the pattern can be an x-y format of features that are in rows and columns. In some embodiments, the pattern can be a repeating arrangement of features and/or interstitial regions. In some embodiments, the pattern can be a random arrangement of features (e.g., clusters) and/or interstitial regions. In some embodiments, the primers are randomly distributed upon the surface. In some embodiments, the primers are distributed on a patterned surface.

In embodiments, the solid support includes a polymer, photoresist or hydrogel layer. In embodiments, the solid support includes a polymer layer. In embodiments, the polymer layer includes polymerized units of alkoxysilyl methacrylate, alkoxysilyl acrylate, alkoxysilyl methylacrylamide, alkoxysilyl methylacrylamide, or a copolymer thereof. In embodiments, the polymer layer includes polymerized units of alkoxysilyl methacrylate. In embodiments, the polymer layer includes polymerized units of alkoxysilyl acrylate. In embodiments, the polymer layer includes polymerized units of alkoxysilyl methylacrylamide. In embodiments, the polymer layer includes polymerized units of alkoxysilyl methylacrylamide. In embodiments, the polymer layer includes glycidyloxypropyl-trimethyloxysilane. In embodiments, the polymer layer includes methacryloxypropyl-trimethoxysilane. In embodiments, the polymer layer includes polymerized units of

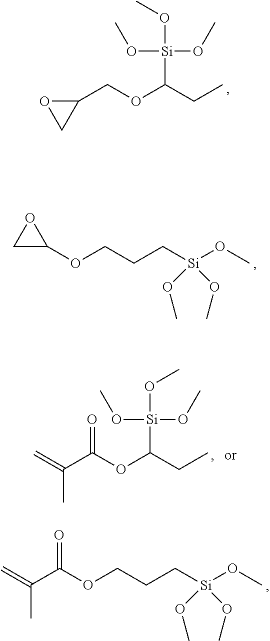

or a copolymer thereof.

In embodiments, the solid support includes a photoresist, alternatively referred to herein as a resist. A “resist” as used herein is used in accordance with its ordinary meaning in the art of lithography and refers to a polymer matrix (e.g., a polymer network). A photoresist is a light-sensitive polymer material used to form a patterned coating on a surface. The process begins by coating a substrate (e.g., a glass substrate) with a light-sensitive organic material. A mask with the desired pattern is used to block light so that only unmasked regions of the material will be exposed to light. In the case of a positive photoresist, the photo-sensitive material is degraded by light and a suitable solvent will dissolve away the regions that were exposed to light, leaving behind a coating where the mask was placed. In the case of a negative photoresist, the photosensitive material is strengthened (either polymerized or cross-linked) by light, and a suitable solvent will dissolve away only the regions that were not exposed to light, leaving behind a coating in areas where the mask was not placed. In embodiments, the solid support includes an epoxy-based photoresist (e.g., SU-8, SU-8 2000, SU-8 3000, SU-8 GLM2060). In embodiments, the solid support includes a negative photoresist. Negative refers to a photoresist whereby the parts exposed to UV become cross-linked (i.e., immobilized), while the remainder of the polymer remains soluble and can be washed away during development. In embodiments, the solid support includes an Off-stoichiometry thiol-enes (OSTE) polymer (e.g., an OSTE resist). In embodiments, the solid support includes an Hydrogen Silsesquioxane (HSQ) polymer (e.g., HSQ resist). In embodiments, the solid support includes a crosslinked polymer matrix on the surface of the wells and the interstitial regions. In embodiments, the photoresist is a silsesquioxane resist, an epoxy-based polymer resist, poly(vinylpyrrolidone-vinyl acrylic acid) copolymer resist, an Off-stoichiometry thiol-enes (OSTE) resist, amorphous fluoropolymer resist, a crystalline fluoropolymer resist, polysiloxane resist, or a organically modified ceramic polymer resist. In embodiments, the photoresist is a silsesquioxane resist. In embodiments, the photoresist is an epoxy-based polymer resist. In embodiments, the photoresist is a poly(vinylpyrrolidone-vinyl acrylic acid) copolymer resist. In embodiments, the photoresist is an Off-stoichiometry thiol-enes (OSTE) resist. In embodiments, the photoresist is an amorphous fluoropolymer resist. In embodiments, the photoresist is a crystalline fluoropolymer resist. In embodiments, the photoresist is a polysiloxane resist. In embodiments, the photoresist is an organically modified ceramic polymer resist. In embodiments, the photoresist includes polymerized alkoxysilyl methacrylate polymers and metal oxides (e.g., SiO₂, ZrO, MgO, Al₂O₃, TiO₂ or Ta₂O₅). In embodiments, the photoresist includes polymerized alkoxysilyl acrylate polymers and metal oxides (e.g., SiO₂, ZrO, MgO, Al₂O₃, TiO₂ or Ta₂O₅). In embodiments, the photoresist includes metal atoms, such as Si, Zr, Mg, Al, Ti or Ta atoms.

In embodiments, the solid support includes a nanoimprint resist. In embodiments, the solid support includes a photoresist and polymer layer, wherein the photoresist is between the solid support and the polymer layer. In embodiments the photoresist is on the interstitial areas and not the surface of the wells. Suitable photoresist compositions are known in the art, such as, for example the compositions and resins described in U.S. Pat. Nos. 6,897,012; 6,991,888; 4,882,245; 7,467,632; 4,970,276, each of which is incorporated herein by reference in their entirety. In embodiments, the solid support includes a photoresist and polymer layer, wherein the photoresist is covalently attached to the solid support and covalently attached to the polymer layer. In embodiments, the resist is an amorphous (non-crystalline) fluoropolymer (e.g., CYTOP® from Bellex), a crystalline fluoropolymer, or a fluoropolymer having both amorphous and crystalline domains. In embodiments, the resist is a suitable polysiloxane, such as polydimethylsiloxane (PDMS). In embodiments, the solid support includes a resist (e.g., a nanoimprint lithography (NIL) resist). Nanoimprint resists can include thermal curable materials (e.g., thermoplastic polymers), and/or UV-curable polymers. In embodiments, the solid support is generated by pressing a transparent mold possessing the pattern of interest (e.g., the pattern of wells) into photo-curable liquid film, followed by solidifying the liquid materials via a UV light irradiation. Typical UV-curable resists have low viscosity, low surface tension, and suitable adhesion to the glass substrate. For example, the solid support surface, but not the surface of the wells, is coated in an organically modified ceramic polymer (ORMOCER®, registered trademark of Fraunhofer-Gesellschaft zur Förderung der angewandten Forschung e. V. in Germany). Organically modified ceramics contain organic side chains attached to an inorganic siloxane backbone. Several ORMOCER® polymers are now provided under names such as “Ormocore”, “Ormoclad” and “Ormocomp” by Micro Resist Technology GmbH. In embodiments, the solid support includes a resist as described in Haas et al Volume 351, Issues 1-2, 30 Aug. 1999, Pages 198-203, US 2015/0079351A1, US 2008/0000373, or US 2010/0160478, each of which is incorporated herein by reference. In embodiments, the solid support surface, and the surface of the wells, is coated in an organically modified ceramic polymer (ORMOCER®, registered trademark of Fraunhofer-Gesellschaft zur Förderung der angewandten Forschung e. V. in Germany). In embodiments, the resist (e.g., the organically modified ceramic polymer) is not removed prior to particle deposition. In embodiments, the wells are within the resist polymer and not the solid support.

In embodiments, the wells are separated from each other by interstitial regions including a polymer layer as described herein (e.g., an amphiphilic copolymer). In embodiments, the solid support further includes a photoresist, wherein the photoresist does not contact the bottom of the well. In embodiments, the polymer layer is substantially free of overlapping amplification clusters. In embodiments, the solid support does not include a polymer (e.g., the solid support is a patterned glass slide). In embodiments, the wells do not include a polymer (e.g., an amphiphilic polymer as described herein). In embodiments, the solid support further includes a photoresist, wherein the photoresist is in contact the bottom of the well and the interstitial space. In embodiments, the wells include a polymer (e.g., an amphiphilic polymer and/or resist as described herein).

In embodiments, each of the platform primers (alternatively referred to herein as primer or polynucleotide primer) is covalently attached to the polymer. In embodiments, the 5′ end of the primer contains a functional group that is tethered to the polymer (i.e., the particle shell polymer or the polymer particle). Non-limiting examples of covalent attachment include amine-modified oligonucleotide moieties on the primer reacting with epoxy or isothiocyanate groups on the polymer, succinylated oligonucleotide moieties on the primer reacting with aminophenyl or aminopropyl functional groups on the polymer, dibenzocycloctyne-modified oligonucleotide moieties on the primer reacting with azide functional groups on the polymer (or vice versa), trans-cyclooctyne-modified oligonucleotide moieties on the primer reacting with tetrazine or methyl tetrazine groups on the polymer (or vice versa), disulfide modified oligonucleotide moieties on the primer reacting with mercapto-functional groups on the polymer, amine-functionalized oligonucleotide moieties on the primer reacting with carboxylic acid groups on the polymer via 1-ethyl-3-(3-dimethylaminopropyl)-carbodiimide hydrochloride (EDC) chemistry, thiol-modified oligonucleotide moieties on the primer attaching to a polymer via a disulfide bond or maleimide linkage, alkyne-modified oligonucleotide moieties on the primer attaching to a polymer via copper-catalyzed click reactions to azide functional groups on the polymer, and acrydite-modified oligonucleotide moieties on the primer polymerizing with free acrylic acid monomers on the polymer to form polyacrylamide or reacting with thiol groups on the polymer. In embodiments, the oligonucleotide moiety on the primer is attached to the polymer through electrostatic binding. For example, the negatively charged phosphate backbone of the primer may be bound electrostatically to positively charged monomers in the polymer. In embodiments, each of the platform primers (alternatively referred to herein as primer or polynucleotide primer) is covalently attached to the solid support via a linker. In embodiments, the linker includes 8 to 16 thymine nucleotides (e.g., consecutive thymine nucleotides, such as a poly-T linker). In embodiments, the linker is at the 5′ end of the immobilized oligonucleotides. In embodiments, the linker includes a cleavable site. In embodiments, the cleavable site includes one or more deoxyuracil nucleobases (dUs). In embodiments, the linker includes 1 to 5 uracil nucleotides.

In embodiments, each platform primer is attached to the polymer, each of which may be present in multiple copies. In embodiments, about or at most 50%, 40%, 30%, 25%, 20%, 15%, 10%, 5%, or less of the polymerized monomers are attached to a platform primer (i.e. a first platform primer, a second platform primer or third platform primer or a complement of any of these thereof). In embodiments, about 1-25%, about 2-20%, about 3-15%, about 4-14%, or about 5-12% of the polymerized monomers are attached to a copy of a platform primer, or a number or a range between any two of these values. In embodiments, about 5-10% of the polymerized monomers are attached to a copy of a platform primer.

In embodiments, each of the platform primers is immobilized on the substrate via a linker. The linker may also include spacer nucleotides. Including spacer nucleotides in the linker puts the polynucleotide in an environment having a greater resemblance to free solution. This can be beneficial, for example, in enzyme-mediated reactions such as sequencing-by-synthesis. It is believed that such reactions suffer less steric hindrance issues that can occur when the polynucleotide is directly attached to the solid support or is attached through a very short linker (e.g., a linker including about 1 to 3 carbon atoms). Spacer nucleotides form part of the polynucleotide but do not participate in any reaction carried out on or with the polynucleotide (e.g. a hybridization or amplification reaction). In embodiments, the spacer nucleotides include 1 to 20 nucleotides. In embodiments, the linker includes 10 spacer nucleotides. In embodiments, the linker includes 12 spacer nucleotides. In embodiments, the linker includes 15 spacer nucleotides. It is preferred to use polyT spacers, although other nucleotides and combinations thereof can be used. In embodiments, the linker includes 10, 11, 12, 13, 14, or 15 T spacer nucleotides. In embodiments, the linker includes 12 T spacer nucleotides. Spacer nucleotides are typically included at the 5′ ends of polynucleotides which are attached to a suitable support. Attachment can be achieved via a phosphorothioate present at the 5′ end of the polynucleotide, an azide moiety, a dibenzocyclooctyne (DBCO) moiety, or any other bioconjugate reactive moiety. The linker may be a carbon-containing chain such as those of formula —(CH₂)n- wherein “n” is from 1 to about 1000. However, a variety of other linkers may be used so long as the linkers are stable under conditions used in DNA sequencing. In embodiments, the linker includes polyethylene glycol (PEG) having a general formula of —(CH₂—CH₂—O)m-, wherein m is from about 1 to 500. In embodiments, m is 8 to 24. In embodiments, m is 10 to 12.

In an aspect is provided a kit, wherein the kit includes the solid support as described herein. In embodiments, the kit includes components necessary to perform the methods as described herein. Generally, the kit includes one or more containers providing a composition and one or more additional reagents (e.g., a buffer suitable for polynucleotide extension). The kit may also include a template nucleic acid (DNA and/or RNA), one or more primer polynucleotides, nucleoside triphosphates (including, e.g., deoxyribonucleotides, ribonucleotides, labeled nucleotides, and/or modified nucleotides), buffers, salts, and/or labels (e.g., fluorophores). In embodiments, the kit includes a solid support (e.g., a patterned substrate such as a flow cell) that includes a plurality of amplification sites, wherein each amplification site includes a population of first platform primers, a population of second platform primers, and a population of third platform primers, wherein each of the third platform primers include a cleavable site as described herein. In embodiments, the kit has each population of platform primers immobilized to the solid support (e.g., the population of first platform primers, the population of second platform primers, and population of third platform primers are each attached to the surface of the solid support). When the solid support includes an array of discrete sites of immobilized oligonucleotides, it may be referred to as an array. In embodiments, the substrate is in a container. The container may be a storage device or other readily usable vessel capable of storing and protecting the substrate.

In embodiments, the kit further includes a first oligonucleotide including a first platform primer binding sequence, a second oligonucleotide including a second platform primer binding sequence, and a third oligonucleotide including a third platform primer binding sequence. In embodiments, the first oligonucleotide includes, from 5′ to 3′, a first platform primer binding sequence, a first sequencing primer binding sequence and optionally an index sequence, wherein the first platform primer binding sequence is complementary to the first platform primer of the amplification site. In embodiments, the second oligonucleotide includes, from 5′ to 3′, a second platform primer binding sequence, a second sequencing primer binding sequence and optionally an index sequence, wherein the second platform primer binding sequence is complementary to the second platform primer of the amplification site. In embodiments, the third oligonucleotide includes, from 5′ to 3′, a third platform primer binding sequence, a second sequencing primer binding sequence and optionally an index sequence, wherein the third platform primer binding sequence is complementary to the third platform primer of the amplification site. In embodiments, the second oligonucleotide and third oligonucleotide include the same sequencing primer binding sequence. In embodiments, the first oligonucleotide and third oligonucleotide include the same sequencing primer binding sequence. In embodiments, the oligonucleotides described above do not include an index sequence.

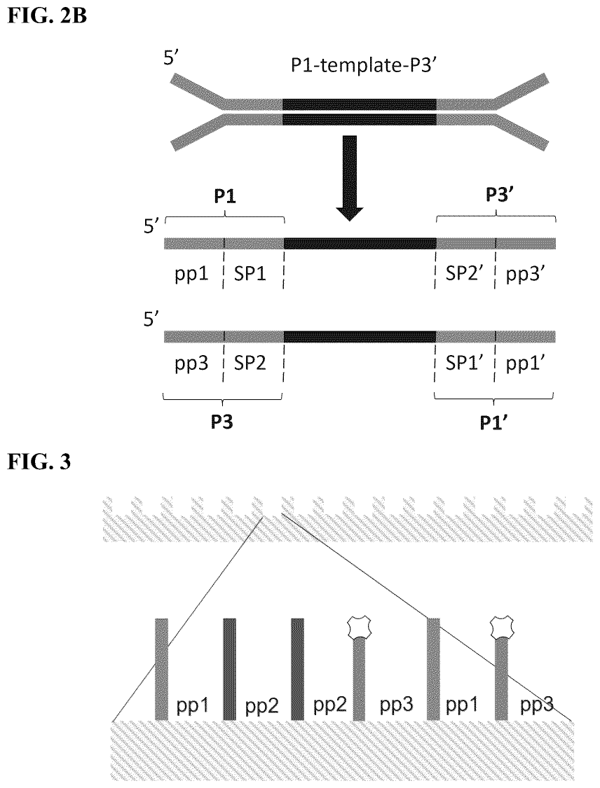

In embodiments, the first oligonucleotide, second oligonucleotide and third oligonucleotide sequences (referred to as P1, P2 and P3, respectively—see FIG. 1) are adapter oligonucleotide sequences that may be attached (e.g. ligated) to sample polynucleotides. For example, a first template polynucleotide includes a first template polynucleotide sequence and a first adapter sequence (P1) attached onto one end of the template polynucleotide sequence and a second adapter sequence on the other end (P2′) attached onto the other end of the template polynucleotide sequence as shown in FIG. 2A. The second oligonucleotide, P2′, includes a second platform primer binding sequence, second sequencing primer binding sequence and optionally an index sequence, and P2′ is complementary to P2. A second template polynucleotide includes a second template polynucleotide sequence, and further includes an adapter sequence (P2) ligated on one end of the template polynucleotide sequence and a different adapter sequence (P1′, wherein P1′ is complementary to P1) attached onto the other end of the polynucleotide sequence as shown in FIG. 2A. In embodiments, the first adapter oligonucleotide sequence (P1) and the second adapter oligonucleotide sequence (P2) include different sequencing primer binding regions (i.e., each has a polynucleotide sequence complementary to a different sequencing primer). In embodiments, the first adapter oligonucleotide sequence and the second adapter oligonucleotide sequence include an index sequence. In embodiments, the first template polynucleotide sequence and second template polynucleotide sequence are complementary to one another.

In embodiments, a third template polynucleotide includes a third template polynucleotide sequence including a first adapter sequence (P1) attached (e.g. ligated) onto one end of the template polynucleotide sequence and a third adapter sequence (P3′), attached onto the other end of the template polynucleotide sequence as shown in FIG. 2B. The third oligonucleotide sequence, P3′, includes a third platform primer binding sequence and second sequencing primer binding sequence, and P3′ is complementary to P3. In embodiments, a fourth template polynucleotide includes a fourth template polynucleotide sequence, and further includes an adapter sequence (P3) attached on one end of the template polynucleotide sequence and a different adapter sequence (P1′), wherein P1′ is complementary to P1 attached onto the other end as shown in FIG. 2B. In embodiments, the first adapter oligonucleotide sequence (P1) and the third adapter oligonucleotide sequence (P3) include different sequencing primer binding regions (i.e., a polynucleotide sequence complementary to a different sequencing primer). In embodiments, P3 has the same sequencing primer binding region as P2. In embodiments, the first adapter sequence and the third adapter sequence include an index sequence. In embodiments, the third template polynucleotide sequence and fourth template polynucleotide sequence are complementary to one another.

In embodiments, the first and second sequencing primer binding sequences are different from each other. In embodiments, the first and third sequencing primer binding sequences are different from each other. In embodiments, the second and third sequencing primer binding sequences are the same as each other. In embodiments, the first and third sequencing primer binding sequences are non-complementary. In embodiments, the first and second sequencing primer binding sequences are non-complementary. In embodiments, the first and second sequencing primer binding sequences each include a different sequence. In embodiments, the first and third sequencing primer binding sequences each include a different sequence. In embodiments, the second and third sequencing primer binding sequences each include the same sequence.

In embodiments, the first oligonucleotide, second oligonucleotide and third oligonucleotide sequences (e.g. P1, P2 and P3, respectively) further include an index sequence (i.e. barcode sequence). In embodiments, the first oligonucleotide, second oligonucleotide and third oligonucleotide sequences further include a barcode sequence that alone or in combination with a sequence of one or both of (a) the sample polynucleotide, or (b) one or more additional barcode sequences, uniquely distinguishing the template polynucleotide from other template polynucleotides in the plurality. In embodiments, each barcode sequence is selected from a set of barcode sequences represented by a random or partially random sequence. In other embodiments, each barcode sequence is selected from a set of barcode sequences represented by a random sequence. In other embodiments, each barcode sequence differs from every other barcode sequence by at least two nucleotide positions. In embodiments, each barcode sequence includes about 5 to about 20 nucleotides, or about 10 to about 20 nucleotides.

In embodiments, the first oligonucleotide, second oligonucleotide and third oligonucleotide sequences (e.g. P1, P2 and P3, respectively) are attached to the template polynucleotide as adapters. In embodiments, two oligonucleotide sequences (e.g., adapter sequences) attach to the template polynucleotide with one on each end of the template polynucleotide. In embodiments, the adapter sequences attached on either end of the template polynucleotide are different (e.g. one end has a P1, the other end has a P2′). In embodiments, an adapter is attached (e.g. ligated) to each end of the nucleic acid fragment (alternatively referred to as a library insert). Ligation of double-stranded DNA adapters may be accomplished by use of T4 DNA ligase. Depending on the adapter, some double-stranded adapters may not have 5′ phosphates and contain a 5′ overhang on one end to prevent ligation in the incorrect orientation. In embodiments, a first adapter is attached (e.g. ligated) to the end of the nucleic acid fragment and second adapter is attached to the end of the nucleic acid fragment. In embodiments, a first adapter is attached to a 5′ end of the nucleic acid fragment and a second adapter is attached to the 3′ end of the nucleic acid fragment. In embodiments, the first adapter sequence includes a first platform primer binding sequence and a first sequencing primer binding sequence and the second adapter sequence includes a second platform primer binding sequence and a second sequencing primer binding sequence. In embodiments, the first platform primer binding sequence is different from the second platform primer binding sequence. In embodiments, the first sequencing primer binding sequence is different from the second sequencing primer binding sequence.

In embodiments, one or more adapters is attached to a plurality of double stranded nucleic acids through ligation. In some embodiments, a first adapter is ligated to a first end of a double stranded nucleic acid, and a second adapter is ligated to a second end of a double stranded nucleic acid. In some embodiments, the first adapter and the second adapter are different. For example, in certain embodiments, the first adapter and the second adapter may include different nucleic acid sequences or different structures (e.g. P1/P2 or P1/P3 or P2/P3). In embodiments, the first adapter and/or second adapter is a Y-adapter. In embodiments, the first adapter and/or second adapter is a hairpin adapter. In some embodiments, the first adapter and/or second adapter is a hairpin adapter and a Y-adapter. In certain embodiments, the first adapter and the second adapter may include different platform primer binding sequences (e.g., a sequence complementary to a capture nucleic acid), different structures, and/or different sequencing primer binding sequences. In embodiments, some, all or substantially all of the nucleic acid sequence of a first adapter and a second adapter are substantially different.

In some embodiments, the template polynucleotide is a double stranded nucleic acid that includes two complementary nucleic acid strands. In certain embodiments, a double stranded nucleic acid includes a first strand and a second strand which are complementary or substantially complementary to each other. A first strand of a double stranded nucleic acid is sometimes referred to herein as a forward strand and a second strand of the double stranded nucleic acid is sometime referred to herein as a reverse strand. In some embodiments, a double stranded nucleic acid includes two opposing ends. Accordingly, a double stranded nucleic acid often includes a first end and a second end. An end of a double stranded nucleic acid may include a 5′-overhang, a 3′-overhang or a blunt end. In some embodiments, one or both ends of a double stranded nucleic acid are blunt ends. In certain embodiments, one or both ends of a double stranded nucleic acid are manipulated to include a 5′-overhang, a 3′-overhang or a blunt end using a suitable method. In some embodiments, one or both ends of a double stranded nucleic acid are manipulated during library preparation such that one or both ends of the double stranded nucleic acid are configured for ligation to an adapter using a suitable method. For example, one or both ends of a double stranded nucleic acid may be digested by a restriction enzyme, polished, end-repaired, filled in, phosphorylated (e.g., by adding a 5′-phosphate), dT-tailed, dA-tailed, the like or a combination thereof.

In embodiments, the double stranded nucleic acid, alternatively referred to as a library insert or template polynucleotide, is at least 50, 100, 150, 200, 250, or 300 nucleotides in length. In embodiments, the double stranded nucleic acid, alternatively referred to as a library insert, is at least 150, 200, 250, 300, 350, or 400 nucleotides in length. In embodiments, the double stranded nucleic acid, alternatively referred to as a library insert, is at least 450, 500, 650, 700, 750, or 800 nucleotides in length. In embodiments, the double stranded nucleic acid, alternatively referred to as a library insert, is at least 850, 900, 950, 1000, 1050, or 1100 nucleotides in length.

In embodiments, the double stranded nucleic acid, alternatively referred to as a library insert, is about 50, 100, 150, 200, 250, or 300 nucleotides in length. In embodiments, the double stranded nucleic acid, alternatively referred to as a library insert, is about 150, 200, 250, 300, 350, or 400 nucleotides in length. In embodiments, the double stranded nucleic acid, alternatively referred to as a library insert, is about 450, 500, 650, 700, 750, or 800 nucleotides in length. In embodiments, the double stranded nucleic acid, alternatively referred to as a library insert, is about 850, 900, 950, 1000, 1050, or 1100 nucleotides in length. In embodiments, the double stranded nucleic acid, alternatively referred to as a library insert, is about 500-1500 nucleotides in length. In embodiments, the double stranded nucleic acid, alternatively referred to as a library insert, is about 750-1500 nucleotides in length. In embodiments, the double stranded nucleic acid, alternatively referred to as a library insert, is about 1-2 kilobases (kb) in length. In embodiments, the double stranded nucleic acid, alternatively referred to as a library insert, is about 300, 400, 600, or 800 nucleotides in length. In embodiments, the double stranded nucleic acid, alternatively referred to as a library insert, is about 250 to 600 nucleotides in length.

In embodiments, ligating includes ligating both the 3′ end and the 5′ end of the duplex region of the first adapter to the double stranded nucleic acid. In embodiments, ligating includes ligating either the 3′ end or the 5′ end of the duplex region of the first adapter to the double stranded nucleic acid. In embodiments, ligating includes ligating the 5′ end of the duplex region of the first adapter to the double stranded nucleic acid and not the 3′ end of the duplex region. In embodiments, the method includes ligating a first adapter to a first end of the double stranded nucleic acid wherein both strands of the double stranded nucleic acid are ligated to the first adapter. In embodiments, the method includes ligating a first adapter to a first end of the double stranded nucleic acid wherein one strand of the double stranded nucleic acid is ligated to the first adapter.

In embodiments, a Y-adapter includes a first strand and a second strand where a portion of the first strand (e.g., 3′-portion) is complementary, or substantially complementary, to a portion (e.g., 5′-portion) of the second strand. In embodiments, a Y-adapter includes a first strand and a second strand where a 3′-portion of the first strand is hybridized to a 5′-portion of the second strand. In embodiments, the 3′-portion of the first strand that is substantially complementary to the 5′-portion of the second strand forms a duplex including double stranded nucleic acid. Accordingly, a Y-adapter often includes a first end including a duplex region including a double stranded nucleic acid, and a second end including a forked region including a 5′-arm and a 3′-arm. In some embodiments, a 5′-portion of the first stand (e.g., 5′-arm) and a 3′-portion of the second strand (3′-arm) are not complementary. In embodiments, the first and second strands of a Y-adapter are not covalently attached to each other. In embodiments, the Y-adapter includes (i) a first strand having a 5′-arm and a 3′-portion, and (ii) a second strand having a 3′-arm and a 5′-portion, wherein the 3′-portion of the first strand is substantially complementary to the 5′-portion of the second strand, and the 5′-arm of the first strand is not substantially complementary to the 3′-arm of the second strand. In some embodiments, the first adapter includes an index sequence, sample barcode sequence or a molecular identifier sequence. In some embodiments, the first adapter includes an index sequence that is a 6-10 nucleotide sequence.

In some embodiments, each strand of a Y-adapter, each of the non-complementary arms of a Y-adapter, or a duplex portion of a Y-adapter has a length independently selected from at least 5, at least 10, at least 15, at least 25, and at least 40 nucleotides. In some embodiments, each strand of a Y-adapter, each of the non-complementary arms of a Y-adapter, or a duplex portion of a Y-adapter has a length in a range independently selected from 15 to 500 nucleotides, 15-250 nucleotides, 15 to 200 nucleotides, 15 to 150 nucleotides, 20 to 100 nucleotides, 20 to 50 nucleotides and 10-50 nucleotides. In embodiments, one or both non-complementary arms of the Y-adapter is about or at least about 10, 15, 20, 25, 30, 35, 40, 45, 50, or more nucleotides in length. In embodiments, one or both non-complementary arms of the Y-adapter is about or at least about 20 nucleotides in length. In embodiments, one or both non-complementary arms of the Y-adapter is about or at least about 30 nucleotides in length. In embodiments, one or both non-complementary arms of the Y-adapter is about or at least about 40 nucleotides in length. In embodiments, the duplex portion of a Y-adapter is about or at least about 5, 10, 15, 20, 25, 30, or more nucleotides in length. In embodiments, the duplex portion of a Y-adapter is about 5-50, 5-25, or 10-15 nucleotides in length. In embodiments, the duplex portion of a Y-adapter is about or at least about 10 nucleotides in length. In embodiments, the duplex portion of a Y-adapter is about or at least about 15 nucleotides in length. In embodiments, the duplex portion of a Y-adapter is about or at least about 12 nucleotides in length. In embodiments, the duplex portion of a Y-adapter is about or at least about 20 nucleotides in length.

In some embodiments, a Y-adapter includes a first end including a duplex region including a double stranded nucleic acid, and a second end including a forked region, where the first end is configured for ligation to an end of a double stranded nucleic acid (e.g., a nucleic acid fragment, e.g., a library insert). In embodiments, a duplex end of a Y-adapter includes a 5′-overhang or a 3′-overhang that is complementary to a 3′-overhang or a 5′-overhang of an end of a double stranded nucleic acid. In some embodiments, a duplex end of a Y-adapter includes a blunt end that can be ligated to a blunt end of a double stranded nucleic acid. In certain embodiment, a duplex end of a Y-adapter includes a 5′-end that is phosphorylated.

In some embodiments, each of the non-complementary portions (i.e., arms) of a Y-adapter independently have a predicted, calculated, mean, average or absolute melting temperature (Tm) that is greater than 50° C., greater than 55° C., greater than 60° C., greater than 65° C., greater than 70° C. or greater than 75° C. In some embodiments, each of the non-complementary portions of a Y-adapter independently have a predicted, estimated, calculated, mean, average or absolute melting temperature (Tm) that is in a range of 50-100° C., 55-100° C., 60-100° C., 65-100° C., 70-100° C., 55-95° C., 65-95° C., 70-95° C., 55-90° C., 65-90° C., 70-90° C., or 60-85° C. In embodiments, the Tm is about or at least about 70° C. In embodiments, the Tm is about or at least about 75° C. In embodiments, the Tm is about or at least about 80° C. In embodiments, the Tm is a calculated Tm. Tm's are routinely calculated by those skilled in the art, such as by commercial providers of custom oligonucleotides. In embodiments, the Tm for a given sequence is determined based on that sequence as an independent oligo. In embodiments, Tm is calculated using web-based algorithms, such as Primer3 and Primer3Plus (www.bioinformatics.nl/cgi-bin/primer3plus/primer3plus.cgi) using default parameters. The Tm of a non-complementary portion of a Y-adapter can be changed (e.g., increased) to a desired Tm using a suitable method, for example by changing (e.g., increasing) GC content, changing (e.g., increasing) length and/or by the inclusion of modified nucleotides, nucleotide analogues and/or modified nucleotides bonds, non-limiting examples of which include locked nucleic acids (LNAs, e.g., bicyclic nucleic acids), bridged nucleic acids (BNAs, e.g., constrained nucleic acids), C5-modified pyrimidine bases (for example, 5-methyl-dC, propynyl pyrimidines, among others) and alternate backbone chemistries, for example peptide nucleic acids (PNAs), morpholinos, the like or combinations thereof. Accordingly, in some embodiments, each of the non-complementary portion of a Y-adapter independently includes one or more modified nucleotides, nucleotide analogues and/or modified nucleotides bonds.

In some embodiments, each of the non-complementary portions of a Y-adapter independently includes a GC content of greater than 40%, greater than 50%, greater than 55%, greater than 60% greater than 65% or greater than 70%. In certain embodiments, each of the non-complementary portions of a Y-adapter independently includes a GC content in a range of 40-100%, 50-100%, 60-100% or 70-100%. In embodiments, one or both non-complementary portions of a Y-adapter have a GC content of about or more than about 40%. In embodiments, one or both non-complementary portions of a Y-adapter have a GC content of about or more than about 50%. In embodiments, one or both non-complementary portions of a Y-adapter have a GC content of about or more than about 60%. Non-base modifiers can also be incorporated into a non-complementary portion of a Y-adapter to increase Tm, non-limiting examples of which include a minor grove binder (MGB), spermine, G-clamp, a Uaq anthraquinone cap, the like or combinations thereof.

In certain embodiments, a duplex region of a Y-adapter includes a predicted, estimated, calculated, mean, average or absolute Tm in a range of 30-70° C., 35-65° C., 35-60° C., 40-65° C., 40-60° C., 35-55° C., 40-55° C., 45-50° C. or 40-50° C. In embodiments, the Tm of a duplex region of the Y-adapter is about or more than about 30° C. In embodiments, the Tm of a duplex region of the Y-adapter is about or more than about 35° C. In embodiments, the Tm of a duplex region of the Y-adapter is about or more than about 40° C. In embodiments, the Tm of a duplex region of the Y-adapter is about or more than about 45° C. In embodiments, the Tm of a duplex region of the Y-adapter is about or more than about 50° C.

In embodiments, a hairpin adapter includes a single nucleic acid strand including a stem-loop structure. A hairpin adapter can be any suitable length. In some embodiments, a hairpin adapter is at least 40, at least 50, or at least 100 nucleotides in length. In some embodiments, a hairpin adapter has a length in a range of 45 to 500 nucleotides, 75-500 nucleotides, 45 to 250 nucleotides, 60 to 250 nucleotides or 45 to 150 nucleotides. In some embodiments, a hairpin adapter includes a nucleic acid having a 5′-end, a 5′-portion, a loop, a 3′-portion and a 3′-end (e.g., arranged in a 5′ to 3′ orientation). In some embodiments, the 5′ portion of a hairpin adapter is annealed and/or hybridized to the 3′ portion of the hairpin adapter, thereby forming a stem portion of the hairpin adapter. In some embodiments, the 5′ portion of a hairpin adapter is substantially complementary to the 3′ portion of the hairpin adapter. In certain embodiments, a hairpin adapter includes a stem portion (i.e., stem) and a loop, wherein the stem portion is substantially double stranded thereby forming a duplex. In some embodiments, the loop of a hairpin adapter includes a nucleic acid strand that is not complementary (e.g., not substantially complementary) to itself or to any other portion of the hairpin adapter. In some embodiments, the second adapter includes an index sequence.

In some embodiments, a duplex region or stem portion of a hairpin adapter includes an end that is configured for ligation to an end of double stranded nucleic acid (e.g., a nucleic acid fragment, e.g., a library insert). In embodiments, an end of a duplex region or stem portion of a hairpin adapter includes a 5′-overhang or a 3′-overhang that is complementary to a 3′-overhang or a 5′-overhang of one end of a double stranded nucleic acid. In some embodiments, an end of a duplex region or stem portion of a hairpin adapter includes a blunt end that can be ligated to a blunt end of a double stranded nucleic acid. In certain embodiment, an end of a duplex region or stem portion of a hairpin adapter includes a 5′-end that is phosphorylated. In some embodiments, a stem portion of a hairpin adapter is at least 15, at least 25, or at least 40 nucleotides in length. In some embodiments, a stem portion of a hairpin adapter has a length in a range of 15 to 500 nucleotides, 15-250 nucleotides, 15 to 200 nucleotides, 15 to 150 nucleotides, 20 to 100 nucleotides or 20 to 50 nucleotides.

In some embodiments, the loop of a hairpin adapter has a predicted, calculated, mean, average or absolute melting temperature (Tm) that is greater than 50° C., greater than 55° C., greater than 60° C., greater than 65° C., greater than 70° C. or greater than 75° C. In some embodiments, a loop of a hairpin adapter has a predicted, estimated, calculated, mean, average or absolute melting temperature (Tm) that is in a range of 50-100° C., 55-100° C., 60-100° C., 65-100° C., 70-100° C., 55-95° C., 65-95° C., 70-95° C., 55-90° C., 65-90° C., 70-90° C., or 60-85° C. In embodiments, the Tm of the loop is about 65° C. In embodiments, the Tm of the loop is about 75° C. In embodiments, the Tm of the loop is about 85° C. The Tm of a loop of a hairpin adapter can be changed (e.g., increased) to a desired Tm using a suitable method, for example by changing (e.g., increasing GC content), changing (e.g., increasing) length and/or by the inclusion of modified nucleotides, nucleotide analogues and/or modified nucleotides bonds, non-limiting examples of which include locked nucleic acids (LNAs, e.g., bicyclic nucleic acids), bridged nucleic acids (BNAs, e.g., constrained nucleic acids), C5-modified pyrimidine bases (for example, 5-methyl-dC, propynyl pyrimidines, among others) and alternate backbone chemistries, for example peptide nucleic acids (PNAs), morpholinos, the like or combinations thereof. Accordingly, in some embodiments, a loop of a hairpin adapter includes one or more modified nucleotides, nucleotide analogues and/or modified nucleotides bonds.

In some embodiments, the loop of a hairpin adapter independently includes a GC content of greater than 40%, greater than 50%, greater than 55%, greater than 60% greater than 65% or greater than 70%. In certain embodiments, a loop of a hairpin adapter independently includes a GC content in a range of 40-100%, 50-100%, 60-100% or 70-100%. In embodiments, the loop has a GC content of about or more than about 40%. In embodiments, the loop has a GC content of about or more than about 50%. In embodiments, the loop has a GC content of about or more than about 60%. Non-base modifiers can also be incorporated into a loop of a hairpin adapter to increase Tm, non-limiting examples of which include a minor grove binder (MGB), spermine, G-clamp, a Uaq anthraquinone cap, the like or combinations thereof. A loop of a hairpin adapter can be any suitable length. In some embodiments, a loop of a hairpin adapter is at least 15, at least 25, or at least 40 nucleotides in length. In some embodiments, a hairpin adapter has a length in a range of 15 to 500 nucleotides, 15-250 nucleotides, 20 to 200 nucleotides, 30 to 150 nucleotides or 50 to 100 nucleotides.

In certain embodiments, a duplex region or stem region of a hairpin adapter includes a predicted, estimated, calculated, mean, average or absolute Tm in a range of 30-70° C., 35-65° C., 35-60° C., 40-65° C., 40-60° C., 35-55° C., 40-55° C., 45-50° C. or 40-50° C. In embodiments, the Tm of the stem region is about or more than about 35° C. In embodiments, the Tm of the stem region is about or more than about 40° C. In embodiments, the Tm of the stem region is about or more than about 45° C. In embodiments, the Tm of the stem region is about or more than about 50° C.

In embodiments, a hairpin structure is formed by joining the ends of a Y-adapter after ligation to a double-stranded nucleic acid. For example, in embodiments disclosed herein relating to ligation to a hairpin adapter, ligation may instead be to a Y-adapter, followed by ligation of the unpaired ends of the adapter to each other. For example, the two unpaired arms may be hybridized to a splint oligonucleotide that brings the ends of the unpaired arms in proximity, which are then ligated with a ligase.

In embodiments, the Y-adaptor portion of a Y-adaptor-ligated double-stranded nucleic acid is formed from cleavage in the loop of a hairpin adapter (e.g., one or more adapters as described in U.S. Pat. No. 8,883,990, which is incorporated herein by reference for all purposes). For example, in embodiments disclosed herein relating to ligation to a Y-adapter, ligation may instead be to a hairpin adapter, followed by cleavage within the loop of the hairpin adapter to release two unpaired ends. In embodiments, a hairpin adapter includes one or more uracil nucleotide(s) in the loop, and cleavage in the loop may be accomplished by the combined activities of Uracil DNA glycosylase (UDG) and the DNA glycosylase-lyase Endonuclease VIII. UDG cleaves the glycosidic bond between the deoxyribose of the DNA sugar-phosphate backbone and the uracil base, and Endonuclease VIII cleaves the AP site, effectively cleaving the loop. In embodiments, the hairpin adapter includes a recognition sequence for a compatible restriction enzyme. In embodiments, the hairpin adapter includes one or more ribonucleotides and cleavage in the loop is accomplished by RNase H. In embodiments, the loop of the hairpin adapter includes a cleavable linkage that is positioned between two non-complementary regions of the loop. In embodiments, the non-complementary region that is 5′ of the cleavable linkage includes a primer binding site that is in the range of 8 to 100 nucleotides in length.

In embodiments, a ligation reaction between the Y adapter, the hairpin adapter, and the DNA fragments is then performed using a suitable ligase enzyme (e.g. T4 DNA ligase) which joins one hairpin adapter and one Y adapter to each DNA fragment, one at either end, to form adapter-target-adapter constructs that somewhat resemble a bobby pin hair fastener. Alternatively, a ligation reaction between a first hairpin adapter, and a different second hairpin adapter, and the DNA fragments is then performed using a suitable ligase enzyme (e.g. T4 DNA ligase) which joins the first hairpin adapter and the second hairpin adapter to each DNA fragment, one at either end, to form adapter-target-adapter constructs.

The products of this reaction can be purified from leftover unligated adapters by a number of means (e.g., NucleoMag NGS Clean-up and Size Select kit, Solid Phase Reversible Immobilization (SPRI) bead methods such as AMPureXP beads, PCRclean-dx kit, Axygen AxyPrep FragmentSelect-I Kit), including size-inclusion chromatography, preferably by electrophoresis through an agarose gel slab followed by excision of a portion of the agarose that contains the DNA greater in size that the size of the adapter. Once formed, the library of adapter-target-adapter templates prepared according to the methods described above can be used for solid-phase nucleic acid amplification.

In embodiments, following ligation, size-selecting and/or purification are performed. By performing a wash, unligated adapters and adapter dimers are removed, and the optimal size-range for subsequent PCR and sequencing is selected. Adapter dimers are the result of self-ligation of the adapters without an insert sequence. These dimers form clusters very efficiently and consume valuable space on the flow cell without generating any useful data. Thus, known cleanup methods may be used, such as magnetic bead-based clean up, or purification on agarose gels.

In some embodiments, the template polynucleotide includes genomic DNA, complementary DNA (cDNA), cell-free DNA (cfDNA), messenger RNA (mRNA), transfer RNA (tRNA), ribosomal RNA (rRNA), cell-free RNA (cfRNA), or noncoding RNA (ncRNA). In embodiments, the template polynucleotide is genomic DNA, complementary DNA (cDNA), cell-free DNA (cfDNA), messenger RNA (mRNA), transfer RNA (tRNA), ribosomal RNA (rRNA), cell-free RNA (cfRNA), or noncoding RNA (ncRNA). In embodiments, the template polynucleotide is genomic DNA. In embodiments, the template polynucleotide is complementary DNA (cDNA). In embodiments, the template polynucleotide is cell-free DNA (cfDNA). In embodiments, the template polynucleotide is messenger RNA (mRNA). In embodiments, the template polynucleotide is transfer RNA (tRNA). In embodiments, the template polynucleotide is ribosomal RNA (rRNA). In embodiments, the template polynucleotide is cell-free RNA (cfRNA). In embodiments, the template polynucleotide is noncoding RNA (ncRNA).

In embodiments, the template polynucleotide is about 20 to 100 nucleotides in length. In embodiments, the template polynucleotide is about 30 to 100 nucleotides in length. In embodiments, the template polynucleotide is about 40 to 100 nucleotides in length. In embodiments, the template polynucleotide is about 50 to 100 nucleotides in length. In embodiments, the template polynucleotide is about 60 to 100 nucleotides in length. In embodiments, the template polynucleotide is about 70 to 100 nucleotides in length. In embodiments, the template polynucleotide is about 80 to 100 nucleotides in length. In embodiments, the template polynucleotide is about 90 to 100 nucleotides in length. In embodiments, the template polynucleotide is about 20 to 200 nucleotides in length. In embodiments, the template polynucleotide is about 30 to 200 nucleotides in length. In embodiments, the template polynucleotide is about 40 to 200 nucleotides in length. In embodiments, the template polynucleotide is about 50 to 200 nucleotides in length. In embodiments, the template polynucleotide is about 60 to 200 nucleotides in length. In embodiments, the template polynucleotide is about 70 to 200 nucleotides in length. In embodiments, the template polynucleotide is about 80 to 200 nucleotides in length. In embodiments, the template polynucleotide is about 90 to 200 nucleotides in length. In embodiments, the template polynucleotide is about 100 to 200 nucleotides in length. In embodiments, the template polynucleotide is less than about 50 nucleotides in length. In embodiments, the template polynucleotide is less than about 75 nucleotides in length. In embodiments, the template polynucleotide is less than about 100 nucleotides in length. In embodiments, the template polynucleotide is less than about 125 nucleotides in length. In embodiments, the template polynucleotide is less than about 150 nucleotides in length. In embodiments, the template polynucleotide is less than about 175 nucleotides in length. In embodiments, the template polynucleotide is less than about 200 nucleotides in length.

In embodiments, the kit includes an array with particles (e.g., particles including immobilized oligonucleotides) optionally loaded into the wells. In embodiments, the array is filled with a buffered solution. Alternatively, in embodiments, the array is not filled with a buffered solution. In embodiments, the array is dry. In embodiments, the array with particles already loaded into the wells is filled with a buffered solution. In embodiments, the particles are in a container. In embodiments, the particles are in aqueous suspension or as a powder within the container. The container may be a storage device or other readily usable vessel capable of storing and protecting the particles.

In embodiments, the kit includes a sequencing polymerase, and one or more amplification polymerases. In embodiments, the sequencing polymerase is capable of incorporating modified nucleotides. In embodiments, the polymerase is a DNA polymerase. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol υ DNA polymerase, or a thermophilic nucleic acid polymerase (e.g., Therminator γ, 9° N polymerase (exo-), Therminator II, Therminator III, or Therminator IX). In embodiments, the DNA polymerase is a thermophilic nucleic acid polymerase. In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044, each of which are incorporated herein by reference for all purposes). In embodiments, the kit includes a strand-displacing polymerase. In embodiments, the kit includes a strand-displacing polymerase, such as a phi29 polymerase, phi29 mutant polymerase or a thermostable phi29 mutant polymerase.

In embodiments, the kit includes a buffered solution. Typically, the buffered solutions contemplated herein are made from a weak acid and its conjugate base or a weak base and its conjugate acid. For example, sodium acetate and acetic acid are buffer agents that can be used to form an acetate buffer. Other examples of buffer agents that can be used to make buffered solutions include, but are not limited to, Tris, bicine, tricine, HEPES, TES, MOPS, MOPSO and PIPES. Additionally, other buffer agents that can be used in enzyme reactions, hybridization reactions, and detection reactions are known in the art. In embodiments, the buffered solution can include Tris. With respect to the embodiments described herein, the pH of the buffered solution can be modulated to permit any of the described reactions. In some embodiments, the buffered solution can have a pH greater than pH 7.0, greater than pH 7.5, greater than pH 8.0, greater than pH 8.5, greater than pH 9.0, greater than pH 9.5, greater than pH 10, greater than pH 10.5, greater than pH 11.0, or greater than pH 11.5. In other embodiments, the buffered solution can have a pH ranging, for example, from about pH 6 to about pH 9, from about pH 8 to about pH 10, or from about pH 7 to about pH 9. In embodiments, the buffered solution can include one or more divalent cations. Examples of divalent cations can include, but are not limited to, Mg²⁺, Mn²⁺, Zn²⁺, and Ca²⁺. In embodiments, the buffered solution can contain one or more divalent cations at a concentration sufficient to permit hybridization of a nucleic acid. In embodiments, the buffered solution can contain one or more divalent cations at a concentration sufficient to permit hybridization of a nucleic acid. In embodiments, the buffered solution includes about 10 mM Tris, about 20 mM Tris, about 30 mM Tris, about 40 mM Tris, or about 50 mM Tris. In embodiments the buffered solution includes about 50 mM NaCl, about 75 mM NaCl, about 100 mM NaCl, about 125 mM NaCl, about 150 mM NaCl, about 200 mM NaCl, about 300 mM NaCl, about 400 mM NaCl, or about 500 mM NaCl. In embodiments, the buffered solution includes about 0.05 mM EDTA, about 0.1 mM EDTA, about 0.25 mM EDTA, about 0.5 mM EDTA, about 1.0 mM EDTA, about 1.5 mM EDTA or about 2.0 mM EDTA. In embodiments, the buffered solution includes about 0.01% Triton X-100, about 0.025% Triton X-100, about 0.05% Triton X-100, about 0.1% Triton X-100, or about 0.5% Triton X-100. In embodiments, the buffered solution includes 20 mM Tris pH 8.0, 100 mM NaCl, 0.1 mM EDTA, 0.025% Triton X-100. In embodiments, the buffered solution includes 20 mM Tris pH 8.0, 150 mM NaCl, 0.1 mM EDTA, 0.025% Triton X-100. In embodiments, the buffered solution includes 20 mM Tris pH 8.0, 300 mM NaCl, 0.1 mM EDTA, 0.025% Triton X-100. In embodiments, the buffered solution includes 20 mM Tris pH 8.0, 400 mM NaCl, 0.1 mM EDTA, 0.025% Triton X-100. In embodiments, the buffered solution includes 20 mM Tris pH 8.0, 500 mM NaCl, 0.1 mM EDTA, 0.025% Triton X-100.

In embodiments, the kit includes one or more sequencing reaction mixtures. In embodiments, the sequencing reaction mixture includes a buffer. In embodiments, the buffer includes an acetate buffer, 3-(N-morpholino)propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), and/or salts (e.g., ammonium sulfate, magnesium chloride, sodium chloride, or potassium chloride).

In embodiments, the kit includes one or more sequencing reaction mixtures. In embodiments, the kit includes one sequencing reaction mixture for each sequencing primer included in the kit (e.g., the kit includes 1, 2, 3, 4, 5, 6, 7, 8, 9, 10, 11, or 12 sequencing reaction mixtures). In embodiments, the kit includes a sequencing reaction mixture including a plurality of different sequencing primer species, wherein all but one of the sequencing primer species is terminated with one or more ddNTPs (e.g., ddCTP, ddATP, ddGTP, or ddTTP) at the 3′ end. In embodiments, a cleavable site is present next to the one or more ddNTPs on the 3′ end, wherein the cleavable site precedes the ddNTPs. In embodiments, the number of different sequencing primer species corresponds to the number of unique adapter sequences and sequencing primer regions present on the template polynucleotides on the surface. For example, if 4 unique sequencing primer binding sites are present on the template polynucleotides, then the sequencing reaction mixture would contain 1 sequencing primer with an extendable 3′ end (e.g., a 3′-OH), and 3 sequencing primers with a cleavable site and one or more ddNTPs at the 3′ end.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay, etc.) from one location to another. For example, kits include one or more enclosures (e.g., boxes) containing the relevant reaction reagents and/or supporting materials. As used herein, the term “fragmented kit” refers to a delivery system including two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the components). The term “kit” includes both fragmented and combined kits. In embodiments, the kit includes, without limitation, nucleic acid primers, probes, adapters, enzymes, and the like, and are each packaged in a container, such as, without limitation, a vial, tube or bottle, in a package suitable for commercial distribution, such as, without limitation, a box, a sealed pouch, a blister pack and a carton. The package typically contains a label or packaging insert indicating the uses of the packaged materials. As used herein, “packaging materials” includes any article used in the packaging for distribution of reagents in a kit, including without limitation containers, vials, tubes, bottles, pouches, blister packaging, labels, tags, instruction sheets and package inserts.

Adapters and/or primers may be supplied in the kits ready for use, as concentrates-requiring dilution before use, or in a lyophilized or dried form requiring reconstitution prior to use. If required, the kits may further include a supply of a suitable diluent for dilution or reconstitution of the primers and/or adapters. Optionally, the kits may further include supplies of reagents, buffers, enzymes, and dNTPs (e.g., dCTP, dATP, dGTP, or dTTP) for use in carrying out nucleic acid amplification and/or sequencing. Further components which may optionally be supplied in the kit include sequencing primers suitable for sequencing templates prepared using the methods described herein.

In an aspect is provided a plurality of template nucleic acids, wherein each template nucleic acid includes a first end, and a second end capable of hybridizing (e.g., via specific hybridization) to any one of the sequences of SEQ ID NO:1 to SEQ ID NO:148, wherein a portion of the plurality of template nucleic acids are different (e.g., different sequences) from each other. In embodiments, the template nucleic acid includes, from 5′ to 3′, a first adapter, a target sequence, and a second adapter. In embodiments more than 50%, or more than 60%, or more than 70%, or more than 80%, or more than 90%, of the plurality of template nucleic acids include different target sequences, wherein substantially all of the template nucleic acids share a common adapter sequence at each end. In embodiments, the first adapter includes a sequence described herein (e.g., a sequence provided in Table 1). In embodiments, the second adapter includes a sequence described herein (e.g., a sequence provided in Table 1), provided the second adapter and first adapter include different sequences.

In embodiments, the oligonucleotides described herein (e.g., the platform primers and/or adapters) include a sequence described in WO2023/034920. In embodiments, the oligonucleotides include a sequence provided in Table 1. For clarity, the sequences in Table 1 do not include any linking spacer nucleotides or cleavable sites.

TABLE 1
Platform primer and/or adapter sequences.
It is understood that white space, line breaks,
and text formatting are not indicative of separate
sequences or structural implications.

| Internal | | |
|---|---|---|
| Ref | | SEQ ID |
| Name | Sequence | Num. |
| RY1 | 5′-CAGGGAAGGAGTGCGTGGCTGCCTTTGT | SEQ ID |
| | | NO: 1 |
| RY2 | 5′-TGTTTCCGTCGGTGCGTGAGGAAGGGAC | SEQ ID |
| | | NO: 2 |
| RY3 | 5′-GTCCCTTCCTCACGCACCGACGGAAACA | SEQ ID |
| | | NO: 3 |
| RY4 | 5′-GTGGTTGGTGAGGGTCATCTCGCTGGAG | SEQ ID |
| | | NO: 4 |
| RY5 | 5′-ACAAAGGCAGCCACGCACTCCTTCCCTG | SEQ ID |
| | | NO: 5 |
| RY6 | 5′-GAGGTCGCTCTACTGGGAGTGGTTGGTG | SEQ ID |
| | | NO: 6 |
| RY7 | 5′-CTCCAGCGAGATGACCCTCACCAACCAC | SEQ ID |
| | | NO: 7 |
| RY8 | 5′-CACCAACCACTCCCAGTAGAGCGACCTC | SEQ ID |
| | | NO: 8 |
| RY9 | 5′-ACAAAGGCAGCCACGCACTCCTTCCCTGAAGGCCGGAATCT | SEQ ID |
| | | NO: 9 |
| RY10 | 5′-GCTGCCGCCACTAGCCATCTTACTGCTGAGGACTCTTCGCT | SEQ ID |
| | | NO: 10 |
| RY11 | 5′-GATTCCGGCCTTGTGGTTGGTGAGGGTCATCTCGCTGGAG | SEQ ID |
| | | NO: 11 |
| RY12 | 5′-GCGAAGAGTCCTGGAGTGCCGCCAATGTATGCGAGGGTGA | SEQ ID |
| | | NO: 12 |
| RY13 | 5′-GCGCGCG TTT TTT TT | SEQ ID |
| | GCTTGCGTCTCCTGCCAGCCATATCCGGTCTACGTGATCC TTT | NO: 13 |
| | TTT TT CGCGCGCT | |
| RY14 | 5′-GCGCGCGTTT TTT TTT TTT TT | SEQ ID |
| | GCTTGCGTCTCCTGCCAGCCATATCCGGTCTACGTGATCC TTT | NO: 14 |
| | TTT TTT TTT TT CGCGCGCT | |
| RY15 | 5′-GGATCACGTAGATTTTGCTTGCGTCTCCTGCCAGCCATATCC | SEQ ID |
| | GGTTTTTCTACGTGATTCCT | NO: 15 |
| RY16 | 5′-GCGAAGAGTCCT GGAGTGCCGCCAATGTATGCGAGGGTGA | SEQ ID |
| | GCTGCCGCCACTAGCCATCTTACTGCTG AGGACTCTTCGCT | NO: 16 |
| RY17 | 5′-GCGAAGAGTCCT TTT TTT | SEQ ID |
| | GGAGTGCCGCCAATGTATGCGAGGGTGA | NO: 17 |
| | GCTGCCGCCACTAGCCATCTTACTGCTG TTT TTT | |
| | AGGACTCTTCGCT | |
| RY18 | 5′-GCGAAGAGTCCT TTT TTT | SEQ ID |
| | GGAGTGCCGCCAATGTATGCGAGGGTGA TTT TTT T | NO: 18 |
| | GCTGCCGCCACTAGCCATCTTACTGCTG TTT TTT | |
| | AGGACTCTTCGCT | |
| RY19 | 5′-GATTCCGGCCTT | SEQ ID |
| | GTGGTTGGTGAGGGTCATCTCGCTGGAGACAAAGGCAGC | NO: 19 |
| | CACGCACTCCTTCCCTGAAGGCCGGAATCT | |
| RY20 | 5′-GATTCCGGCCTT TTT TTT | SEQ ID |
| | GTGGTTGGTGAGGGTCATCTCGCTGGAGACAAAGGCAGCCACGC | NO: 20 |
| | ACTCCTTCCCTG TTTTTT AAGGCCGGAATCT | |
| RY21 | 5′-GATTCCGGCCTT TTT TTT | SEQ ID |
| | GTGGTTGGTGAGGGTCATCTCGCTGGAGTTT TTT | NO: 21 |
| | TACAAAGGCAGCCACGCACTCCTTCCCTG TTT TTT | |
| | AAGGCCGGAATCT | |
| RY22 | 5′-GGATCACGTAGATTTTGCTTGCGTCTCCTGCCAGCCATAT | SEQ ID |
| | CCGGTTTTTCTACGTGATCCT | NO: 22 |
| RY23 | 5′-GG ATC ACG TAG ATT TTT TTT TTT TGC TTG CGT CTC CTG | SEQ ID |
| | CCA GCC ATA TCC GGT TTT TTT TTT TTT CTA CGT GAT CCT | NO: 23 |
| RY24 | 5′-GG ATC ACG TAG ATT TTT TTT TTT TTT TTT TTT TTT TGC TTG | SEQ ID |
| | CGT CTC CTG CCA GCC ATA TCC GGT TTT TTT TTT TTT TTT TTT | NO: 24 |
| | TTT TTT CTA CGT GAT CCT | |
| RY25 | 5′-GG ATC ACG TAG ATT TTT TTT TTT TTT TTT TTT TTT TTT TTT | SEQ ID |
| | TTT TTT TTT TTG CTT GCG TCT CCT GCC AGC CAT ATC CGG TTT | NO: 25 |
| | TTT TTT TTC TAC GTG ATC CT | |
| RY26 | 5′-GGA TCA CGT AGA TTT TAG ATC TGC TTG CGT CTC CTG CCA | SEQ ID |
| | GCC ATA TCC GGT TTT TCT ACG TGA TCC T | NO: 26 |
| RY27 | 5′-GGA TCA CGT AGA TTTTTTTTTTTT AGA TCT GCT TGC GTC | SEQ ID |
| | TCC TGC CAG CCA TAT CCG GTTTTTTTTTTTTC TAC GTG ATC CT | NO: 27 |
| RY28 | 5′-TGTTTCCGTCGGTGCGTGAGGAAGGGACTTCCGGCCTTAGA | SEQ ID |
| | | NO: 28 |
| RY29 | 5′-CGACGGCGGTGATCGGTAGAATGACGACTCCTGAGAAGCGA | SEQ ID |
| | | NO: 29 |
| RY30 | 5′-CTAAGGCCGGAACACCAACCACTCCCAGTAGAGCGACCTC | SEQ ID |
| | | NO: 30 |
| RY31 | 5′-CGCTTCTCAGGACCTCACGGCGGTTACATACGCTCCCACT | SEQ ID |
| | | NO: 31 |
| RY32 | 5′-CGCGCGCAAAAAAAACGAACGCAGAGGACG | SEQ ID |
| | GTCGGTATAGGCCAGATGCACTAGGAAAAAAAAGCGCGCGA | NO: 32 |
| RY33 | 5′-CGCGCGCAAAAAAAAAAAAAACGAACGCAG | SEQ ID |
| | AGGACGGTCGGTATAGGCCAGATGCACTAGGAAAAAAAAAAAAA | NO: 33 |
| | AGCGCGCGA | |
| RY34 | 5′-CCTAGTGCATCTAAAACGAACGCAGAGGAC | SEQ ID |
| | GGTCGGTATAGGCCAAAAAGATGCACTAAGGA | NO: 34 |
| RY35 | 5′-CGCTTCTCAGGACCTCACGGCGGTTACATACG | SEQ ID |
| | CTCCCACTCGACGGCGGTGATCGGTAGAATGACGACTCCTGAGAA | NO: 35 |
| | GCGA | |
| RY36 | 5′-CGCTTCTCAGGAAAAAAACCTCACGGCGGT | SEQ ID |
| | TACATACGCTCCCACTCGACGGCGGTGATCGGTAGAATGAC | NO: 36 |
| | GACAAAAAATCCTGAGAAGCGA | |
| RY37 | 5′- | SEQ ID |
| | CGCTTCTCAGGAAAAAAACCTCACGGCGGTTACATACGCTCCCAC | NO: 37 |
| | TAAAAAAACGACGGCGGTGATCGGTAGAATGACGACAAAAAATC | |
| | CTGAGAAGCGA | |
| RY38 | 5′- | SEQ ID |
| | CTAAGGCCGGAACACCAACCACTCCCAGTAGAGCGACCTCTGTTT | NO: 38 |
| | CC GTCGGTGCGTGAGGAAGGGACTTCCGGCCTTAGA | |
| RY39 | 5′- | SEQ ID |
| | CTAAGGCCGGAAAAAAAACACCAACCACTCCCAGTAGAGCGACC | NO: 39 |
| | TC | |
| | TGTTTCCGTCGGTGCGTGAGGAAGGGACAAAAAATTCCGGCCTTA | |
| | GA | |
| RY40 | 5′- | SEQ ID |
| | CTAAGGCCGGAAAAAAAACACCAACCACTCCCAGTAGAGCGACC | |
| | TCAA | |
| | AAAAATGTTTCCGTCGGTGCGTGAGGAAGGGACAAAAAATTCCG | NO: 40 |
| | GCCTTAGA | |
| RY41 | 5′- | SEQ ID |
| | CCTAGTGCATCTAAAACGAACGCAGAGGACGGTCGGTATAGGCC | NO: 41 |
| | AAA AAGATGCACTAGGA | |
| RY42 | 5′- | SEQ ID |
| | CCTAGTGCATCTAAAAAAAAAAAACGAACGCAGAGGACGGTCGG | NO: 42 |
| | TAT AGGCCAAAAAAAAAAAAAGATGCACTAGGA | |
| RY43 | 5′-CCTAGTGCATCTAAAAAAAAAAAAAAAAAAA | SEQ ID |
| | AAAAACGAACGCAGAGGACGGTCGGTATAGGCCAAAAAAAAAA | NO: 43 |
| | A AAAAAAAAAAAAAAGATGCACTAGGA | |
| RY44 | 5′-CCTAGTGCATCTAAAAAAAAAAAAAAAAAAAAA | SEQ ID |
| | AAAAAAAAAAAAAAAAAAACGAACGCAGAGGACGGTCGGTATA | NO: 44 |
| | GGCCA AAAAAAAAAAGATGCACTAGGA | |
| RY45 | 5′- | SEQ ID |
| | CCTAGTGCATCTAAAATCTAGACGAACGCAGAGGACGGTCGGTA | NO: 45 |
| | TAGGCCAAAAAGATGCACTAGGA | |
| RY46 | 5′- | SEQ ID |
| | CCTAGTGCATCTAAAAAAAAAAAATCTAGACGAACGCAGAGGAC | NO: 46 |
| | G GTCGGTATAGGCCAAAAAAAAAAAAGATGCACTAGGA | |
| RY47 | 5′-AGATTCCGGCCTTCAGGGAAGGAGTGCGTGGCTGCCTTTGT | SEQ ID |
| | | NO: 47 |
| RY48 | 5-AGCGAAGAGTCCTCAGCAGTAAGATGGCTAGTGGCGGCAGC | SEQ ID |
| | | NO: 48 |
| RY49 | 5′-TCACCCTCGCATACATTGGCGGCACTCCAGGACTCTTCGC | SEQ ID |
| | | NO: 49 |
| RY50 | 5′-CTCCAGCGAGATGACCCTCACCAACCACAAGGCCGGAATC | SEQ ID |
| | | NO: 50 |
| RY51 | 5′- | SEQ ID |
| | AGCGCGCGAAAAAAAAGGATCACGTAGACCGGATATGGCTGGCA | NO: 51 |
| | GGAGACGCAAGCAAAAAAAACGCGCGC | |
| RY52 | 5′-AGGAATCACGTAGAAAAACCGGATATGGCTGGCAGGAG | SEQ ID |
| | ACGCAAGCAAAATCTACGTGATCC | NO: 52 |
| RY53 | 5′-AGCGCGCGAAAAAAAAAAAAAAGGATCACGTAGACCG | SEQ ID |
| | GATATGGCTGGCAGGAGACGCAAGCAAAAAAAAAAAAAACGCG | NO: 53 |
| | CGC | |
| RY54 | 5′-AGCGAAGAGTCCTCAGCAGTAAGATGGCTAGTGGCGGC | SEQ ID |
| | AGCTCACCCTCGCATACATTGGCGGCACTCCAGGACTCTTCGC | NO: 54 |
| RY55 | 5′-AGCGAAGAGTCCTAAAAAACAGCAGTAAGATGGCTAG | SEQ ID |
| | TGGCGGCAGCTCACCCTCGCATACATTGGCGGCACTCCAAAAAAA | NO: 55 |
| | GGACTCTTCGC | |
| RY56 | 5′- | SEQ ID |
| | AGCGAAGAGTCCTAAAAAACAGCAGTAAGATGGCTAGTGGCGGC | NO: 56 |
| | AGCAAAAAAATCACCCTCGCATACATTGGCGGCACTCCAAAAAA | |
| | AGGACTCTTCGC | |
| RY57 | 5′-AGATTCCGGCCTTCAGGGAAGGAGTGCGTGGCTGCCTTTGTCTC | SEQ ID |
| | CAGCGAGATGACCCTCACCAACCACAAGGCCGGAATC | NO: 57 |
| RY58 | 5′- | SEQ ID |
| | AGATTCCGGCCTTAAAAAACAGGGAAGGAGTGCGTGGCTGCCTTT | NO: 58 |
| | GTCTCCAGCGAGATGACCCTCACCAACCACAAAAAAAAGGCCGG | |
| | AATC | |
| RY59 | 5′- | SEQ ID |
| | AGATTCCGGCCTTAAAAAACAGGGAAGGAGTGCGTGGCTGCCTT | NO: 59 |
| | TGTAAAAAAACTCCAGCGAGATGACCCTCACCAACCACAAAAAA | |
| | AAGGCCGGAATC | |
| RY60 | 5′- | SEQ ID |
| | AGGATCACGTAGAAAAACCGGATATGGCTGGCAGGAGACGCAAG | NO: 60 |
| | C AAAATCTACGTGATCC | |
| RY61 | 5′- | SEQ ID |
| | AGGATCACGTAGAAAAAAAAAAAAACCGGATATGGCTGGCAGGA | NO: 61 |
| | GACGCAAGCAAAAAAAAAAAATCTACGTGATCC | |
| RY62 | 5′- | SEQ ID |
| | AGGATCACGTAGAAAAAAAAAAAAAAAAAAAAAAAAACCGGAT | |
| | AT | |
| | GGCTGGCAGGAGACGCAAGCAAAAAAAAAAAAAAAAAAAAAAA | NO: 62 |
| | ATCTACGTGATCC | |
| RY63 | 5′- | SEQ ID |
| | AGGATCACGTAGAAAAAAAAAAAACCGGATATGGCTGGCAGGAG | NO: 63 |
| | AC GCAAGCAGATCTAAAAAAAAAAAATCTACGTGATCC | |
| RY64 | 5′- | SEQ ID |
| | AGGATCACGTAGAAAAACCGGATATGGCTGGCAGGAGACGCAAG | NO: 64 |
| | CA GATCTAAAATCTACGTGATCC | |
| RY65 | 5′- | SEQ ID |
| | AGGATCACGTAGAAAAAAAAAAACCGGATATGGCTGGCAGGAGA | |
| | CGC | |
| | AAGCAAAAAAAAAAAAAAAAAAAAAAAAAAAAAAAAAAAAAA | NO: 65 |
| | AATCTACGTGATCC | |
| RY66 | 5′-AGTGGGAGCGTATGTAACCGCCGTGAGGTCCTGAGAAGCG | SEQ ID |
| | | NO: 66 |
| RY67 | 5′-GAGGTCGCTCTACTGGGAGTGGTTGGTGTTCCGGCCTTAG | SEQ ID |
| | | NO: 67 |
| RY68 | 5′-TCGCTTCTCAGGAGTCGTCATTCTACCGATCACCGCCGTCG | SEQ ID |
| | | NO: 68 |
| RY69 | 5′-TCTAAGGCCGGAAGTCCCTTCCTCACGCACCGACGGAAACA | SEQ ID |
| | | NO: 69 |
| RY70 | 5′- | SEQ ID |
| | TCCTTAGTGCATCTTTTTGGCCTATACCGACCGTCCTCTGCGTTCG | NO: 70 |
| | T TTTAGATGCACTAGG | |
| RY71 | 5′- | SEQ ID |
| | TCGCGCGCTTTTTTTTTTTTTTCCTAGTGCATCTGGCCTATACCGAC | NO: 71 |
| | C GTCCTCTGCGTTCGTTTTTTTTTTTTTTGCGCGCG | |
| RY72 | 5′- | SEQ ID |
| | TCGCGCGCTTTTTTTTCCTAGTGCATCTGGCCTATACCGACCGTCC | NO: 72 |
| | T CTGCGTTCGTTTTTTTTGCGCGCG | |
| RY73 | 5′- | SEQ ID |
| | TCGCTTCTCAGGAGTCGTCATTCTACCGATCACCGCCGTCGAGTG | NO: 73 |
| | G GAGCGTATGTAACCGCCGTGAGGTCCTGAGAAGCG | |
| RY74 | 5′- | SEQ ID |
| | TCGCTTCTCAGGATTTTTTGTCGTCATTCTACCGATCACCGCCGTC | NO: 74 |
| | G | |
| | TTTTTTTAGTGGGAGCGTATGTAACCGCCGTGAGGTTTTTTTCCTG | |
| | AGAAGCG | |
| RY75 | 5′- | SEQ ID |
| | TCGCTTCTCAGGATTTTTTGTCGTCATTCTACCGATCACCGCCGTC | NO: 75 |
| | GAG | |
| | TGGGAGCGTATGTAACCGCCGTGAGGTTTTTTTCCTGAGAAGCG | |
| RY76 | 5′- | SEQ ID |
| | TCTAAGGCCGGAATTTTTTGTCCCTTCCTCACGCACCGACGGAAA | |
| | CAT | |
| | TTTTTTGAGGTCGCTCTACTGGGAGTGGTTGGTGTTTTTTTTCCGG | NO: 76 |
| | CCTTAG | |
| RY77 | 5′- | SEQ ID |
| | TCTAAGGCCGGAATTTTTTGTCCCTTCCTCACGCACCGACGGAAA | NO: 77 |
| | CAG | |
| | AGGTCGCTCTACTGGGAGTGGTTGGTGTTTTTTTTCCGGCCTTAG | |
| RY78 | 5′- | SEQ ID |
| | TCTAAGGCCGGAAGTCCCTTCCTCACGCACCGACGGAAACAGAG | NO: 78 |
| | GT CGCTCTACTGGGAGTGGTTGGTGTTCCGGCCTTAG | |
| RY79 | 5′- | SEQ ID |
| | TCCTAGTGCATCTTTTTTTTTTTTTTTTTTTTTTTTTGGCCTATACCG | NO: 79 |
| | ACC | |
| | GTCCTCTGCGTTCGTTTTTTTTTTTTTTTTTTTTTTTTAGATGCACTA | |
| | GG | |
| RY80 | 5′- | SEQ ID |
| | TCCTAGTGCATCTTTTTTTTTTTTTGGCCTATACCGACCGTCCTCTG | NO: 80 |
| | CG TTCGTTTTTTTTTTTTAGATGCACTAGG | |
| RY81 | 5′- | SEQ ID |
| | TCCTAGTGCATCTTTTTGGCCTATACCGACCGTCCTCTGCGTTCGT | NO: 81 |
| | TTTAGATGCACTAGG | |
| RY82 | 5′- | SEQ ID |
| | TCCTAGTGCATCTTTTTTTTTTTTGGCCTATACCGACCGTCCTCTGC | NO: 82 |
| | GTTCGTCTAGATTTTTTTTTTTTAGATGCACTAGG | |
| RY83 | 5′- | SEQ ID |
| | TCCTAGTGCATCTTTTTGGCCTATACCGACCGTCCTCTGCGTTCGT | NO: 83 |
| | C TAGATTTTAGATGCACTAGG | |
| RY84 | 5′- | SEQ ID |
| | TCCTAGTGCATCTTTTTTTTTTTGGCCTATACCGACCGTCCTCTGC | NO: 84 |
| | G | |
| | TTCGTTTTTTTTTTTTTTTTTTTTTTTTTTTTTTTTTTTTTTTTAGATG | |
| | CACTAGG | |
| RY85 | 5′-ACAAAGGCAGCCACGCACTCCTTCC | SEQ ID |
| | | NO: 85 |
| RY86 | 5′-CTCCAGCGAGATGACC | SEQ ID |
| | | NO: 86 |
| RY87 | 5′-CTCCAGCGAGATGACCCTCACCAAC | SEQ ID |
| | | NO: 87 |
| RY88 | 5′-ACAAAGGCAGCCACGC | SEQ ID |
| | | NO: 88 |
| RY89 | 5′-CTCCAGCGAGATGACCCTCACC | SEQ ID |
| | | NO: 89 |
| RY90 | 5′-ACAAAGGCAGCCACGCACT | SEQ ID |
| | | NO: 90 |
| RY91 | 5′-CTCCAGCGAGATGACCCTC | SEQ ID |
| | | NO: 91 |
| RY92 | 5′-ACAAAGGCAGCCACGCACTCCT | SEQ ID |
| | | NO: 92 |
| RY93 | 5′-CCTTCCTCACGCACCGACGGAAACA | SEQ ID |
| | | NO: 93 |
| RY94 | 5′-CAACCACTCCCAGTAGAGCGACCTC | SEQ ID |
| | | NO: 94 |
| RY95 | 5′-TCCTCACGCACCGACGGAAACA | SEQ ID |
| | | NO: 95 |
| RY96 | 5′-CCACTCCCAGTAGAGCGACCTC | SEQ ID |
| | | NO: 96 |
| RY97 | 5′-TCACGCACCGACGGAAACA | SEQ ID |
| | | NO: 97 |
| RY98 | 5′-CTCCCAGTAGAGCGACCTC | SEQ ID |
| | | NO: 98 |
| RY99 | 5′-CGCACCGACGGAAACA | SEQ ID |
| | | NO: 99 |
| RY100 | 5′-CCAGTAGAGCGACCTC | SEQ ID |
| | | NO: 100 |
| RY101 | 5′-GGAAGGAGTGCGTGGCTGCCTTTGT | SEQ ID |
| | | NO: 101 |
| RY102 | 5′-GTTGGTGAGGGTCATCTCGCTGGAG | SEQ ID |
| | | NO: 102 |
| RY103 | 5′-AGGAGTGCGTGGCTGCCTTTGT | SEQ ID |
| | | NO: 103 |
| RY104 | 5′-GGTGAGGGTCATCTCGCTGGAG | SEQ ID |
| | | NO: 104 |
| RY105 | 5′-AGTGCGTGGCTGCCTTTGT | SEQ ID |
| | | NO: 105 |
| RY106 | 5′-GAGGGTCATCTCGCTGGAG | SEQ ID |
| | | NO: 106 |
| RY107 | 5′-GCGTGGCTGCCTTTGT | SEQ ID |
| | | NO: 107 |
| RY108 | 5′-GGTCATCTCGCTGGAG | SEQ ID |
| | | NO: 108 |
| RY109 | 5′-TGTTTCCGTCGGTGCGTGAGGAAGG | SEQ ID |
| | | NO: 109 |
| RY110 | 5′-GAGGTCGCTCTACTGGGAGTGGTTG | SEQ ID |
| | | NO: 110 |
| RY111 | 5′-TGTTTCCGTCGGTGCGTGAGGA | SEQ ID |
| | | NO: 111 |
| RY112 | 5′-GAGGTCGCTCTACTGGGAGTGG | SEQ ID |
| | | NO: 112 |
| RY113 | 5′-TGTTTCCGTCGGTGCGTGA | SEQ ID |
| | | NO: 113 |
| RY114 | 5′-GAGGTCGCTCTACTGGGAG | SEQ ID |
| | | NO: 114 |
| RY115 | 5′-TGTTTCCGTCGGTGCG | SEQ ID |
| | | NO: 115 |
| RY116 | 5′-GAGGTCGCTCTACTGG | SEQ ID |
| | | NO: 116 |
| RY117 | 5′-AAGGCAGCCACGCACTCCTTCCCTG | SEQ ID |
| | | NO: 117 |
| RY118 | 5′-CAGCGAGATGACCCTCACCAACCAC | SEQ ID |
| | | NO: 118 |
| RY119 | 5′-GCAGCCACGCACTCCTTCCCTG | SEQ ID |
| | | NO: 119 |
| RY120 | 5′-CGAGATGACCCTCACCAACCAC | SEQ ID |
| | | NO: 120 |
| RY121 | 5′-GCCACGCACTCCTTCCCTG | SEQ ID |
| | | NO: 121 |
| RY122 | 5′-GATGACCCTCACCAACCAC | SEQ ID |
| | | NO: 122 |
| RY123 | 5′-ACGCACTCCTTCCCTG | SEQ ID |
| | | NO: 123 |
| RY124 | 5′-GACCCTCACCAACCAC | SEQ ID |
| | | NO: 124 |
| RY125 | 5′-GTCCCTTCCTCACGCACCGACGGAA | SEQ ID |
| | | NO: 125 |
| RY126 | 5′-CACCAACCACTCCCAGTAGAGCGAC | SEQ ID |
| | | NO: 126 |
| RY127 | 5′-GTCCCTTCCTCACGCACCGACG | SEQ ID |
| | | NO: 127 |
| RY128 | 5′-CACCAACCACTCCCAGTAGAGC | SEQ ID |
| | | NO: 128 |
| RY129 | 5′-GTCCCTTCCTCACGCACCG | SEQ ID |
| | | NO: 129 |
| RY130 | 5′-CACCAACCACTCCCAGTAG | SEQ ID |
| | | NO: 130 |
| RY131 | 5′-GTCCCTTCCTCACGCA | SEQ ID |
| | | NO: 131 |
| RY132 | 5′-CACCAACCACTCCCAG | SEQ ID |
| | | NO: 132 |
| RY133 | 5′-CAGGGAAGGAGTGCGTGGCTGCCTT | SEQ ID |
| | | NO: 133 |
| RY134 | 5′-GTGGTTGGTGAGGGTCATCTCGCTG | SEQ ID |
| | | NO: 134 |
| RY135 | 5′-CAGGGAAGGAGTGCGTGGCTGC | SEQ ID |
| | | NO: 135 |
| RY136 | 5′-GTGGTTGGTGAGGGTCATCTCG | SEQ ID |
| | | NO: 136 |
| RY137 | 5′-CAGGGAAGGAGTGCGTGGC | SEQ ID |
| | | NO: 137 |
| RY138 | 5′-GTGGTTGGTGAGGGTCATC | SEQ ID |
| | | NO: 138 |
| RY139 | 5′-CAGGGAAGGAGTGCGT | SEQ ID |
| | | NO: 139 |
| RY140 | 5′-GTGGTTGGTGAGGGTC | SEQ ID |
| | | NO: 140 |
| RY141 | 5′-TTCCGTCGGTGCGTGAGGAAGGGAC | SEQ ID |
| | | NO: 141 |
| RY142 | 5′-GTCGCTCTACTGGGAGTGGTTGGTG | SEQ ID |
| | | NO: 142 |
| RY143 | 5′-CGTCGGTGCGTGAGGAAGGGAC | SEQ ID |
| | | NO: 143 |
| RY144 | 5′-GCTCTACTGGGAGTGGTTGGTG | SEQ ID |
| | | NO: 144 |
| RY145 | 5′-CGGTGCGTGAGGAAGGGAC | SEQ ID |
| | | NO: 145 |
| RY146 | 5′-CTACTGGGAGTGGTTGGTG | SEQ ID |
| | | NO: 146 |
| RY147 | 5′-TGCGTGAGGAAGGGAC | SEQ ID |
| | | NO: 147 |
| RY148 | 5′-CTGGGAGTGGTTGGTG | SEQ ID |
| | | NO: 148 |
| RY149 | 5′-ACG ACC TTC TTG TAG TCC TTA CGG C | SEQ ID |
| | | NO: 170 |
| RY150 | 5′-ACA GTT TAG GTC CAC TCT CCA CCA C | SEQ ID |
| | | NO: 171 |
| RY151 | 5′-TGA TAG CTG AAA CTA GCC TCA CCG C | SEQ ID |
| | | NO: 172 |
| RY152 | 5′-ACC CAT ATC GAG GAG TCA AGT TGG C | SEQ ID |
| | | NO: 173 |
| RY153 | 5′-ATG GGC TGC CTA TGC CGT AAT ATC C | SEQ ID |
| | | NO: 174 |
| RY154 | 5′-AGT AAT GAA CAG CGC GTG GTC ACA C | SEQ ID |
| | | NO: 175 |

In an aspect is provided a composition including a solid support and one, two, three, or more different pluralities of immobilized oligonucleotides, wherein the oligonucleotides in each plurality each include a sequence described herein (e.g., a sequence in Table 1). In embodiments, the sequence is selected from SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:170, SEQ ID NO:171, SEQ ID NO:172, SEQ ID NO:173, SEQ ID NO:174, or SEQ ID NO:175. In embodiments, the oligonucleotides in each plurality each include a sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, SEQ ID NO:121, SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:170, SEQ ID NO:171, SEQ ID NO:172, SEQ ID NO:173, SEQ ID NO:174, or SEQ ID NO:175, provided each plurality of oligonucleotides includes a different sequence.

In embodiments, the oligonucleotide includes a sequencing primer binding sequence (e.g., 5′-AGATCGGAAGAGCACACGTCTGAACTCCAGTCA (SEQ ID NO:149), 5′-AGATCGGAAGAGCGTCGTGTAGGGAAAGAGTGT (SEQ ID NO:150), 5′-GCCTTGGCACCCGAGAATTCCA (SEQ ID NO:151), 5′-ACACTCTTTCCCTACACGACGCTCTTCCGATCT (SEQ ID NO:152), 5′-CACTCTTTCCCTACACGACGCTCTTCCGATCT (SEQ ID NO:153), 5′-CGGTCTCGGCATTCCTGCTGAACCGCTCTTCCGATCT (SEQ ID NO: 154), 5′-ACTCTTTCCCTACACGACGCTCTTCCGATCT (SEQ ID NO:155), 5′-GTGACTGGAGTTCAGACGTGTGCTCTTCCGATCT (SEQ ID NO:156), 5′-CAAGCAGAAGACGGCATACGA (SEQ ID NO:157), 5′-CGACTCACTATAGGGAGAGCGGC (SEQ ID NO:158), 5′-AAGAACATCGATTTTCCATGGCAG (SEQ ID NO:159), 5′-AACGCCAAACCTACGGCTTTACTTCCTGTGGCT (SEQ ID NO:160), 5′-TCTTGAGTCATTCGCAGGGCATGTGCCAGACCT (SEQ ID NO:161), 5′-TCGGCGTTGTCTGCTATCGTTCTTGGCACTCCT (SEQ ID NO:162), 5′-GGAGCAATAACCATAAGGCCGTTGACAAGCCCT (SEQ ID NO:163), 5′-GGCGTATTGCCTTGGTTCTGGCAGCCTCATTGT (SEQ ID NO:164), 5′-CAGCAGAGGGAACGATTTCAACTTCCTGTGGCT (SEQ ID NO:165), 5′-CTACTGCAAGGGTGTCTAGAATGTGCCAGACCT (SEQ ID NO:166), 5′-GACCGACTCGTGAAACGTAATCTTGGCACTCCT (SEQ ID NO:167), 5′-ACACATTCTTTGCGCCCAGAGTTGACAAGCCCT (SEQ ID NO:168), 5′-ATTTCATTCGACACCCGGTCGCAGCCTCATTGT (SEQ ID NO:169), or a complement thereof). In embodiments, the oligonucleotide further includes an index sequence (e.g., a barcode or UMI). In embodiments, the index sequence includes 1, 2, 3, 4, 5, 6, 7, 8, 9, 10, 11, or 12 nucleotides. In embodiments, the index sequence is 5 nucleotides. In embodiments, the index sequence is 6 nucleotides. In embodiments, the index sequence is 8 nucleotides. In embodiments, the index sequence is 12 nucleotides. In general, the index is of sufficient length and includes sequences that are sufficiently different to allow the identification of associated features or nucleic acid sequences based on barcodes with which they are associated.

### III. Methods

In an aspect is provided a method of amplifying a polynucleotide on a solid support including a plurality of immobilized primers, the method including hybridizing a second platform primer binding sequence of a first immobilized polynucleotide to a second immobilized primer; wherein the first immobilized polynucleotide includes a first platform primer sequence immobilized to a solid support, a template sequence, and the second platform primer binding sequence; hybridizing a third platform primer binding sequence of a second immobilized polynucleotide to a third immobilized primer including a cleavable site; wherein the second immobilized polynucleotide includes the first platform primer sequence, a template sequence, and the third platform primer binding sequence; extending the second immobilized primer with a polymerase to form a first amplification product and extending the third immobilized primer with a polymerase to form a second amplification product including the cleavable site; cleaving the cleavable site and removing the second amplification product; and amplifying the first amplification product and the first immobilized polynucleotide.

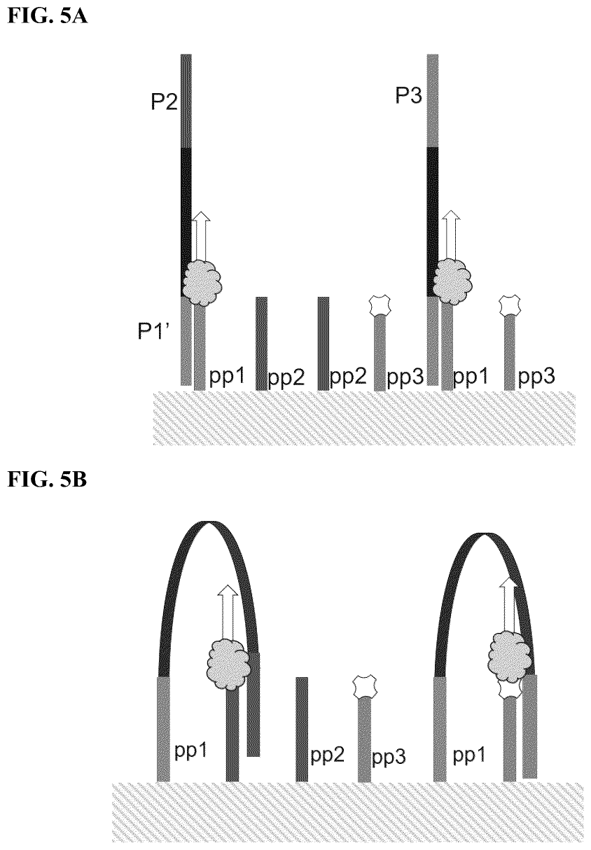

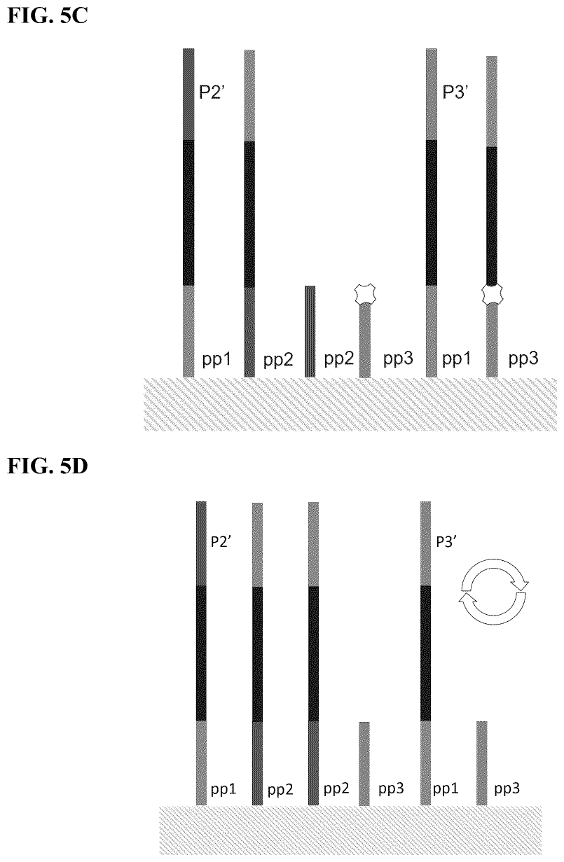

In embodiments, the first immobilized polynucleotide includes a first platform primer sequence immobilized to a solid support, a template sequence or a complement thereof, and a second platform primer binding sequence that hybridizes to a second immobilized primer (e.g., pp2 as shown in FIG. 5B). In embodiments, second immobilized primer is extended with a polymerase to form a first amplification product (e.g. a template sequence or complement thereof attached to the immobilized platform pp2 on one end, and adapter P1′ or complement thereof attached to the other end, as shown in FIG. 5C). In embodiments, the second amplification product (e.g. a template sequence including immobilized pp3 and adapter P1′ or complement thereof, as shown in FIG. 5C) includes a cleavable site. The cleavable site is a site which allows controlled cleavage of the polynucleotide strand by chemical, enzymatic, or photochemical means. In embodiments, the cleavable site includes a diol linker, disulfide linker, photocleavable linker, abasic site, deoxyuracil triphosphate (dUTP), deoxy-8-Oxo-guanine triphosphate (d-8-oxoG), methylated nucleotide, ribonucleotide, or a sequence containing a modified or unmodified nucleotide that is specifically recognized by a cleaving agent. In embodiments, the cleavable site includes one or more deoxyuracil nucleobases (dUs). In embodiments, the cleavable site includes one or more ribonucleotides. In embodiments, the cleavable site includes 2 to 5 ribonucleotides. In embodiments, the cleavable site includes one ribonucleotide. In embodiments, the cleavable site includes more than one ribonucleotide. In embodiments, the cleavable site includes deoxyuracil triphosphate (dUTP) or deoxy-8-oxo-guanine triphosphate (d-8-oxoG).

Any suitable enzymatic, chemical, or photochemical cleavage reaction may be used to cleave the cleavable site. Suitable cleavage means include, for example, restriction enzyme digestion, in which case the cleavage site is an appropriate restriction site for the enzyme which directs cleavage of a portion of the primer; RNase digestion or chemical cleavage of a bond between a deoxyribonucleotide and a ribonucleotide, in which case the cleavage site may include one or more ribonucleotides; chemical reduction of a disulfide linkage with a reducing agent (e.g., THPP or TCEP), in which case the cleavage site should include an appropriate disulfide linkage; chemical cleavage of a diol linkage with periodate, in which case the cleavage site should include a diol linkage; generation of an abasic site and subsequent hydrolysis, etc. In embodiments, the cleavage site is included in the oligonucleotide (e.g., within the oligonucleotide sequence of the third platform primer which becomes part of the second amplification product). In embodiments, the linker or the oligonucleotide includes a diol linkage which permits cleavage by treatment with periodate (e.g., sodium periodate). It will be appreciated that more than one diol can be included at the cleavage site. One or more diol units may be incorporated into a polynucleotide using standard methods for automated chemical DNA synthesis. Oligonucleotide nucleotide primers including one or more diol linkers can be conveniently prepared by chemical synthesis. The diol linker is cleaved by treatment with any substance which promotes cleavage of the diol (e.g., a diol-cleaving agent). In embodiments, the diol-cleaving agent is periodate, e.g., aqueous sodium periodate (NaIO₄). Following treatment with the diol-cleaving agent (e.g., periodate) to cleave the diol, the cleaved product may be treated with a “capping agent” in order to neutralize reactive species generated in the cleavage reaction. Suitable capping agents for this purpose include amines, e.g., ethanolamine or propanolamine. In embodiments, cleavage may be accomplished by using a modified nucleotide as the cleavable site (e.g., uracil, 8oxoG, 5-mC, 5-hmC) that is removed or nicked via a corresponding DNA glycosylase, endonuclease, or combination thereof.

In embodiments, cleaving the cleavable site includes contacting the cleavable site with a cleaving agent. In embodiments, the cleaving agent is selected from sodium periodate, RNase, formamidopyrimidine DNA glycosylase (Fpg), endonuclease, uracil DNA glycosylase (UDG), TCEP, THPP, sodium dithionite (Na₂S₂O₄), hydrazine (N₂H₄), Pd(0), or ultraviolet radiation.

In embodiments, cleavage of the cleavable site, which includes a modified nucleotide, for example, one or more uracils, may be accomplished using a cleavage mixture including about 150 mM to about 300 mM glycine-KOH, about 5 mM to about 15 mM MgCl2, about 0.05% to about 0.15% Triton X-100, and about 0.05 U/μL to about 0.2 U/μL uracil DNA glycosylase (UDG). In embodiments, the cleavage mixture can have a pH greater than pH 8.0, greater than pH 8.5, greater than pH 9.0, greater than pH 9.5, or greater than pH 10.0. In other embodiments, the cleavage mixture can have a pH ranging, for example, from about pH 8.0 to about pH 10.0, from about pH 8.5 to about pH 10.0, or from about pH 9.0 to about pH 10.0. For example, the cleavage mixture is applied to an immobilized oligonucleotide (i.e. a third platform primer) including one or more uracils, incubated at about 37° C. to about 42° C. for 10 min, and then incubated at about 65° C. to about 72° C. for 30 min. Following cleavage, the surface is washed with wash buffer, followed by subsequent washes with about 0.05M NaOH to about 0.15M NaOH, and optionally another wash with wash buffer.

In embodiments, following cleavage of the cleavage site, the second amplification product is removed from the solid support (as shown in FIG. 5D). In embodiments, removal of the second amplification product does not include removal of all or a portion of the immobilized platform primer sequence (e.g., pp3). In embodiments, the second amplification products may be removed from a surface or substrate using a suitable method, for example by restriction enzyme cleavage. Any restriction enzyme or any enzyme restriction site known to a skilled artisan can be used in a method or composition provided herein. For example, the restriction endonuclease can be a Type I enzyme (EC 3.1.21.3), a Type II enzyme (EC 3.1.21.4), a Type III enzyme (EC 3.1.21.5), or a Type IV enzyme (EC 3.1.21.5). Restriction endonucleases can include, for example, without limitation, Alu I, Ava I, Bam HI, Bgl II, Eco P15 I, Eco RI, Eco RII, Eco RV, Hae III, Hga I, Hha I, Hind III, Hinf I, Hpa I, Kpn I, Mbo I, Not I, Pst I, Pvu II, Sac I, Sal I, SapI, Sau 3A, Sca I, Sma I, Spe I, Sph I, Sst I, Stu I, Taq I, Xba I or Xma I. Cleaving one strand of a duplex may be referred to as linearization. Suitable methods for linearization are known and described in more detail in U.S. Patent Publication No. 2009/0118128, which is incorporated herein by reference in its entirety. For example, the second amplification product may be cleaved by exposing the second amplification product to a mixture containing a glycosylase and one or more suitable endonucleases. In embodiments, cleaving includes chemically cleaving one strand of the second amplification product at a cleavable site. In embodiments, the cleavable site includes a diol linker, disulfide linker, photocleavable linker, abasic site, deoxyuracil triphosphate (dUTP), deoxy-8-Oxo-guanine triphosphate (d-8-oxoG), methylated nucleotide, ribonucleotide, or a sequence containing a modified or unmodified nucleotide that is specifically recognized by a cleaving agent.

In embodiments, the method includes removing immobilized primers that do not contain a first or second strand of the nucleic acid template (i.e., unused primers) on a solid support. Methods of removing immobilized primers can include digestion using an enzyme with exonuclease activity. Removing unused primers may serve to increase the free volume and allow for greater accessibility. Removal of unused primers may also prevent opportunities for the newly released first strand to rehybridize to an available surface primer, producing a priming site off the available surface primer, thereby facilitating the “reblocking” of the released first strand.

In embodiments, following the removal of the second amplification product from the substrate, the other remaining set of substrate-attached amplicons is subjected to further amplification (e.g., as depicted in FIG. 5D).

In embodiments, the amplifying is at discrete locations in an ordered array of amplification sites on the surface. In some embodiments, the surface does not include an ordered array of amplification sites. For example, the surface may be uniformly coated with platform primers, rather than coating some areas (amplification sites) and not others (interstitial regions).

In embodiments, amplifying includes incubation in a denaturant. In embodiments, the denaturant is acetic acid, ethylene glycol, hydrochloric acid, nitric acid, formamide, guanidine, sodium salicylate, sodium hydroxide, dimethyl sulfoxide (DMSO), propylene glycol, urea, or a mixture thereof. In embodiments, the denaturant is an additive that lowers a DNA denaturation temperature. In embodiments, the denaturant is betaine, dimethyl sulfoxide (DMSO), ethylene glycol, formamide, glycerol, guanidine thiocyanate, 4-methylmorpholine 4-oxide (NMO), or a mixture thereof. In embodiments, the denaturant is betaine, dimethyl sulfoxide (DMSO), ethylene glycol, formamide, glycerol, guanidine thiocyanate, or 4-methylmorpholine 4-oxide (NMO).

In embodiments, amplifying includes a plurality of cycles of strand denaturation, primer hybridization, and primer extension. Although each cycle will include each of these three events (denaturation, hybridization, and extension), events within a cycle may or may not be discrete. For example, each step may have different reagents and/or reaction conditions (e.g., temperatures). Alternatively, some steps may proceed without a change in reaction conditions. For example, extension may proceed under the same conditions (e.g., same temperature) as hybridization. After extension, the conditions are changed to start a new cycle with a new denaturation step, thereby amplifying the amplicons. Primer extension products from an earlier cycle may serve as templates for a later amplification cycle. In embodiments, the plurality of cycles is about 5 to about 50 cycles. In embodiments, the plurality of cycles is about to about 45 cycles. In embodiments, the plurality of cycles is about 10 to about 20 cycles. In embodiments, the plurality of cycles is about 20 to about 30 cycles. In embodiments, the plurality of cycles is 10 to 45 cycles. In embodiments, the plurality of cycles is 10 to 20 cycles. In embodiments, the plurality of cycles is 20 to 30 cycles. In embodiments, the plurality of cycles is about 10 to about 45 cycles. In embodiments, the plurality of cycles is about 20 to about cycles.

In embodiments, amplifying includes bridge polymerase chain reaction (bPCR) amplification, solid-phase rolling circle amplification (RCA), solid-phase exponential rolling circle amplification (eRCA), solid-phase recombinase polymerase amplification (RPA), solid-phase helicase dependent amplification (HDA), template walking amplification, emulsion PCR, or combinations thereof. In embodiments, amplifying includes a bridge polymerase chain reaction (bPCR) amplification. In embodiments, amplifying includes a thermal bridge polymerase chain reaction (t-bPCR) amplification. In embodiments, amplifying includes a chemical bridge polymerase chain reaction (c-bPCR) amplification. Chemical bridge polymerase chain reactions include fluidically cycling a denaturant (e.g., formamide) and maintaining the temperature within a narrow temperature range (e.g., +/−5° C.). In contrast, thermal bridge polymerase chain reactions include thermally cycling between high temperatures (e.g., 85° C.-95° C.) and low temperatures (e.g., 60° C.-70° C.). Thermal bridge polymerase chain reactions may also include a denaturant, typically at a much lower concentration than traditional chemical bridge polymerase chain reactions. Solid phase recombinase polymerase amplification (RPA) utilizes recombinase proteins that interact with primers present in a sample mixture to create a recombinase primer complex that reads target DNA and binds accordingly. The recombinase primer complex separates the hydrogen bonds between the two strands of nucleotides of the DNA and replaces them with the complementary regions of the recombinase primer complex, allowing amplification without using fluctuating temperatures to displace adjacent strands. Additionally, helicase dependent amplification (HDA) does not require thermocycling as a DNA helicase generates single-stranded templates for primer hybridization and subsequent primer extension is done by a DNA polymerase. Template walking amplification is also an isothermal amplification process based on a template walking mechanism and utilizes low-melting temperature solid-surface homopolymer primers and solution phase primer. In template walking amplification, hybridization of a primer to a template strand is followed by primer extension to form a first extended strand, partial or incomplete denaturation of the extended strand from the template strand. Primer extension in subsequence amplification cycles then involve displacement of first extended strand from the template strand.

In embodiments, amplifying includes 1 to 100 cycles of solid-phase rolling circle amplification (RCA), solid-phase exponential rolling circle amplification (eRCA), solid-phase recombinase polymerase amplification (RPA), solid-phase helicase dependent amplification (HDA), or template walking amplification. In embodiments, amplifying includes 1 to 100 thermal bridge polymerase chain reaction (t-bPCR) amplification, chemical bridge polymerase chain reaction (c-bPCR) amplification or chemical-thermal bridge polymerase chain reaction (cT-bPCR) amplification).

In embodiments, a bridge PCR amplification method produces a first set of amplicons that are complementary to an original template, and a second set of amplicons that have nucleic acid sequences substantially identical to the original template, where both the first and second sets of amplicons are attached to a substrate (e.g., a substrate of a flow cell). In embodiments, amplifying includes 1 to 100 bridge-PCR amplification cycles. In embodiments, amplifying includes a first subset (e.g., 1 to 25) bridge-PCR amplification cycles, cleaving the cleavable site and removing the second amplification product, followed by a second subset of amplification cycles (e.g., an additional 1 to 25) bridge-PCR amplification cycles. In embodiments, the first subset includes 5-20 cycles of bridge-PCR and the second subset includes to 80 cycles of bridge-PCR amplification.

In embodiments, amplifying results in higher ratio of first immobilized polynucleotide and first amplification product relative to the second immobilized polynucleotide and second amplification product. In embodiments, the first immobilized polynucleotide and first amplification product are confined to an area of a discrete region (referred to as a cluster).

In embodiments, the cluster is monoclonal (i.e., one template polynucleotide (e.g., a first template polynucleotide) binds and is amplified within the feature). In embodiments, the cluster is polyclonal (i.e., more than one template polynucleotide type (e.g., a first template polynucleotide and a second template polynucleotide) binds and is amplified within the feature). In embodiments, the array contains a ratio of monoclonal (e.g., one template polynucleotide (e.g., a first template polynucleotide)), diclonal (e.g., two template polynucleotides (e.g., a first and a second template polynucleotide)), triclonal (e.g., three template polynucleotides (e.g., a first, second, and a third template polynucleotide)), quadraclonal (e.g., four template polynucleotides (e.g., a first, second, third, and fourth template polynucleotide)), etc. clusters. In embodiments, multiple different template polynucleotides seed one spot (i.e., a feature) of a patterned array, and is referred to herein as a polyclonal feature. In embodiments, a fraction of the surface area within the feature is occupied by copies of one template type, and another fraction of the patterned spot can be occupied by copies of another template type (e.g., a first template polynucleotide and a second template polynucleotide, wherein each template polynucleotide is different). The fractions of the template polynucleotides within the feature are inherently stochastic and governed by Poisson statistics, however the ratios may be influenced by underseeding or overseeding (i.e., providing less or more template polynucleotides relative to the number of available sites on the array) as well as cleavage of the cleavage sites on the third platform primers. In some embodiments, the ratio of monoclonal amplification clusters to polyclonal amplification clusters is at least about 1:1. In some embodiments, the ratio of monoclonal amplification clusters to polyclonal amplification clusters is at least about 2:1. In embodiments, the ratio of monoclonal amplification clusters to polyclonal amplification clusters is at least about 3:1. In embodiments, the ratio of monoclonal amplification clusters to polyclonal amplification clusters is at least about 1.0:1.0 to 3.0:1.0 or any number within this range. In embodiments, the ratio of monoclonal amplification clusters to polyclonal amplification clusters is at least about 1.5:1. In embodiments, the ratio of monoclonal amplification clusters to polyclonal amplification clusters is at least about 2.5:1. Clonality generally refers to a population of nucleic acids that is homogeneous with respect to a particular nucleotide sequence. For example, the homogenous sequence can be at least 10 nucleotides long, or longer, for example, at least 50, 100, or 250 nucleotides in length. A clonal population can be derived from a single target nucleic acid or template nucleic acid. In embodiments, substantially all of the nucleic acids in a monoclonal population have the same nucleotide sequence. It will be understood that a small number of mutations (e.g., due to amplification artifacts) can occur in a monoclonal population without departing from monoclonality.

In embodiments, the template polynucleotide (e.g., genomic template DNA) is first treated to form single-stranded linear fragments (e.g., ranging in length from about 50 to about 600 nucleotides). Treatment typically entails fragmentation, such as by chemical fragmentation, enzymatic fragmentation, or mechanical fragmentation, followed by denaturation to produce single-stranded DNA fragments. In embodiments, the template polynucleotide includes an adapter. The adaptor may have other functional elements including tagging sequences (i.e., a barcode), attachment sequences, palindromic sequences, restriction sites, sequencing primer binding sites, functionalization sequences, and the like. Barcodes can be of any of a variety of lengths. In embodiments, the primer includes a barcode that is 10-50, 20-30, or 4-12 nucleotides in length. In embodiments, the adapter includes a primer binding sequence that is complementary to at least a portion of a primer (e.g., a sequencing primer). Primer binding sites can be of any suitable length. In embodiments, a primer binding site is about or at least about 10, 15, 20, 25, 30, or more nucleotides in length. In embodiments, a primer binding site is 10-50, 15-30, or 20-25 nucleotides in length. The primer binding site may be selected such that the primer (e.g., sequencing primer) has the following properties, for example having a length of about 20-30 nucleotides; approximately 50% GC content, and a Tm of about 55° C. to about 65° C.

In embodiments, the array includes 30% monoclonal clusters relative to total amplification sites. In embodiments, the array includes 50% monoclonal clusters relative to total amplification sites. In embodiments, the array includes 30% to 50% monoclonal clusters relative to total amplification sites or any number within the range (e.g. 31%, 32%, etc.). In embodiments, the array includes 30%, 35%, 40%, 45% or 50% monoclonal clusters relative to total amplification sites. In some embodiments, fewer than 50% of all of the clusters are monoclonal amplification clusters. In some embodiments, fewer than 45% of all of the clusters are monoclonal amplification clusters. In some embodiments, fewer than 40% of all of the clusters are monoclonal amplification clusters. In some embodiments, fewer than 35% of all of the clusters are monoclonal amplification clusters. In some embodiments, fewer than 30% of all of the clusters are monoclonal amplification clusters. In some embodiments, fewer than 25% of all of the clusters are monoclonal amplification clusters.

In another aspect is provided a method of forming a first immobilized polynucleotide and a second immobilized polynucleotide on a solid support, the method including: contacting a solid support with a first polynucleotide and a second polynucleotide, wherein the solid support includes a population of first platform primers, a population of second platform primers, and a population of third platform primers, wherein each third platform primer includes a cleavable site and wherein each of the first platform primers, the second platform primers and the third platform primers are immobilized to the solid support; hybridizing a first platform primer binding sequence of the first polynucleotide to one of the first platform primers, wherein the first polynucleotide includes the first platform primer binding sequence, a template sequence, and a second platform primer sequence; hybridizing a first platform primer binding sequence of the second polynucleotide to one of the first platform primers, wherein the second polynucleotide includes the first platform primer binding sequence, a template sequence, and a third platform primer sequence; extending the first platform primer with a polymerase to form the first immobilized polynucleotide including the first platform primer sequence, a complement of the template sequence, and a second platform primer binding sequence; and extending the second platform primer with a polymerase to form the second immobilized polynucleotide including the first platform primer sequence, a complement of the template sequence, and a third platform primer binding sequence.

In some embodiments, the method includes hybridizing an adapter attached to a template sequence (e.g. a nucleic acid template), wherein the adapter includes a sequence complementary to a platform primer (i.e. capture nucleic acid) immobilized to a solid support. In certain embodiments, attaching a nucleic acid template to a substrate includes annealing a platform primer (i.e. capture nucleic acid) to a template. In some embodiments, a platform primer anneals to a complementary sequence that is present on an adapter portion of a template (e.g., a Y-adapter or hairpin adapter). In certain embodiments, a platform primer anneals to a primer binding site located on a Y-adapter portion of a template described herein. A platform primer may anneal to a portion of a Y-adapter on or near the 3′-end or 3′-side of a template. In some embodiments, a platform primer anneals to a 3′-arm of a Y-adapter on a template.

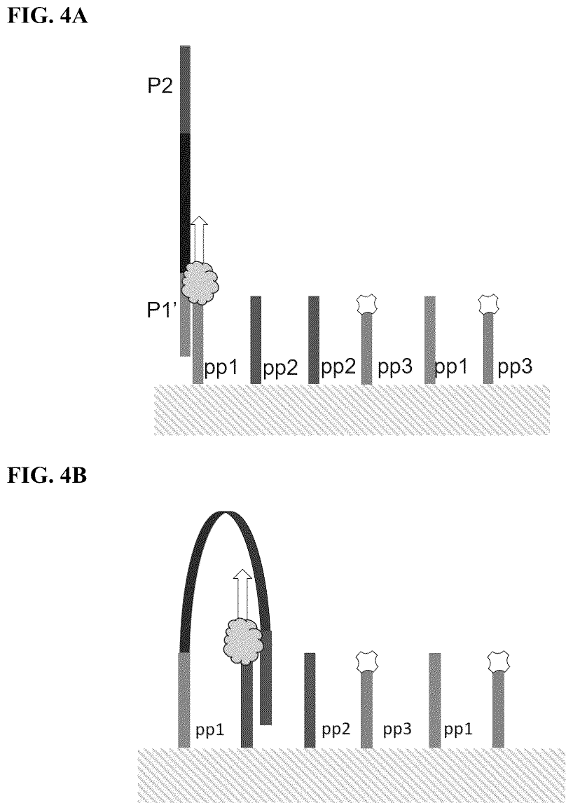

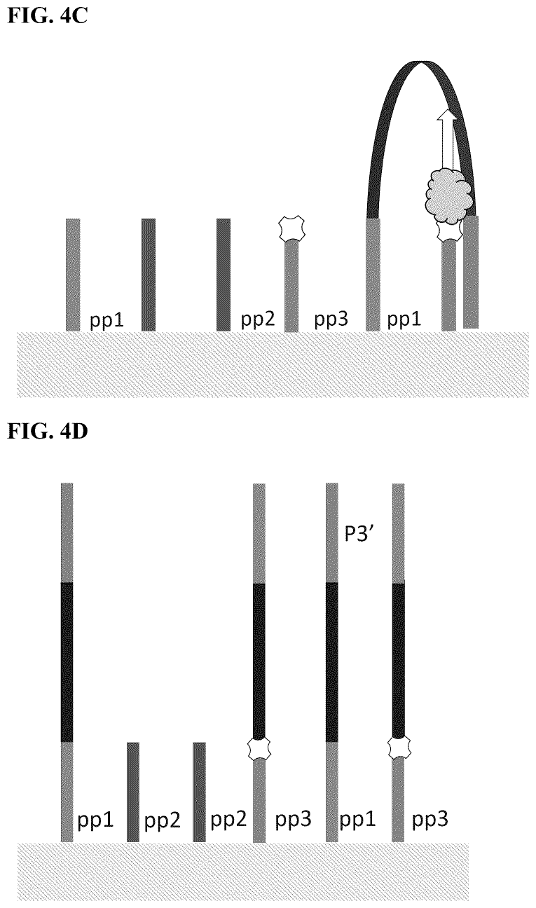

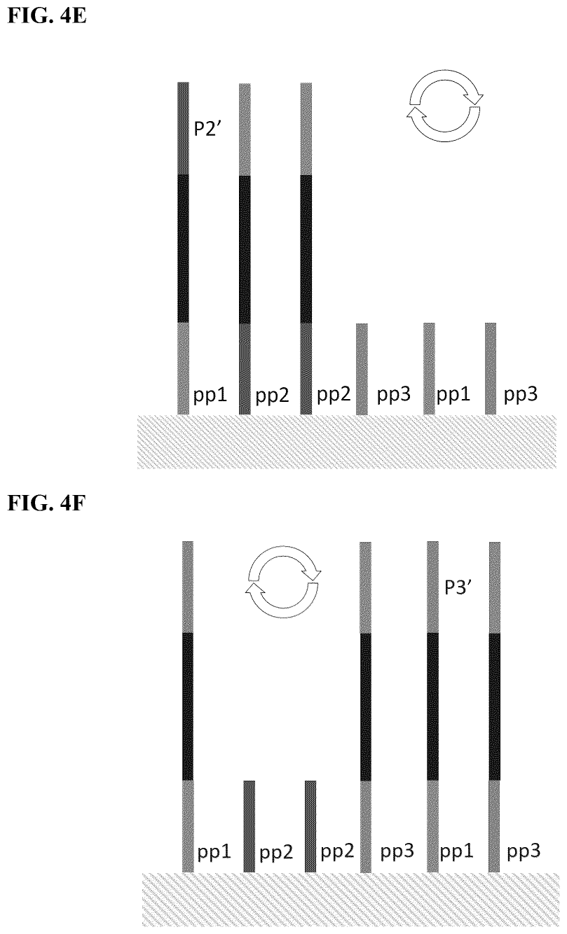

In embodiments, the first immobilized polynucleotide is formed when the first platform primer binding sequence of a first polynucleotide that includes the first platform primer binding sequence, a template sequence or complement thereof, and a second platform primer sequence hybridizes to a first immobilized platform primer and is extended with a polymerase (e.g., as shown in FIG. 4A) to generate a first immobilized polynucleotide. In embodiments, the first immobilized polynucleotide includes the first platform primer sequence or complement thereof (e.g., pp1) immobilized to the solid support, the template sequence or complement thereof and a second platform primer binding sequence complementary to an immobilized second platform primer (e.g. as shown in FIG. 4B). In embodiments, the second immobilized polynucleotide is formed when the first platform primer binding sequence of a second polynucleotide that includes the first platform primer binding sequence, a template sequence or complement thereof, and a third platform primer sequence hybridizes to a first immobilized platform primer and is extended with a polymerase to generate a second immobilized polynucleotide. In embodiments, the second immobilized polynucleotide (as depicted in FIG. 4C) includes the first platform primer sequence or complement thereof (e.g. pp1) immobilized to the solid support, the template sequence or complement thereof and a third platform primer binding sequence (e.g. contained within P3′) complementary to an immobilized third platform primer that includes a cleavable site (e.g. as shown in FIG. 4C).

In embodiments, the second platform primer binding sequence of the first immobilized polynucleotide hybridizes to an immobilized second platform primer and the second platform primer and is extended with a polymerase (as depicted in FIG. 4B) to form a first amplification product (shown in FIG. 4E). The first amplification product includes the immobilized second platform primer, template sequence or complement thereof and a first platform primer binding sequence or complement thereof. In embodiments, the third platform primer binding sequence of the second immobilized polynucleotide hybridizes to a third platform primer immobilized to the solid support and is extended with a polymerase (e.g. as depicted in FIG. 4C) to form a second amplification product (e.g. as shown in FIG. 4D) that has the immobilized third platform primer including the cleavable site, the template sequence or complement thereof, and a first platform primer binding sequence (or complement thereof).

In embodiments, following formation of the first amplification product and second amplification product (as shown in FIG. 5C), the cleavable site is cleaved. In embodiments, cleaving of the cleavable site causes the second amplification product to cleave so that the third platform primer remains immobilized to the solid support while the rest of the second amplification strand is no longer immobilized to the solid support (see FIG. 5D). In embodiments, the second amplification product not immobilized to the solid support is removed from the solid support. Following cleavage of the cleavable site, further amplification is performed to form a plurality of immobilized extension products (as depicted in FIG. 5D). The cleavable site is a site which allows controlled cleavage of the polynucleotide strand by chemical, enzymatic, or photochemical means. In embodiments, the cleavable site includes a diol linker, disulfide linker, photocleavable linker, abasic site, deoxyuracil triphosphate (dUTP), deoxy-8-Oxo-guanine triphosphate (d-8-oxoG), methylated nucleotide, ribonucleotide, or a sequence containing a modified or unmodified nucleotide that is specifically recognized by a cleaving agent. In embodiments, the cleavable site includes one or more deoxyuracil nucleobases (dUs). In embodiments, the cleavable site includes one or more ribonucleotides. In embodiments, the cleavable site includes 2 to 5 ribonucleotides. In embodiments, the cleavable site includes one ribonucleotide. In embodiments, the cleavable site includes more than one ribonucleotide. In embodiments, the cleavable site includes deoxyuracil triphosphate (dUTP) or deoxy-8-oxo-guanine triphosphate (d-8-oxoG). The cleavable site can be cleaved using methods described herein.

After removal of one of the sets of amplicons from the substrate, the other remaining set of substrate-attached amplicons is subjected to further amplification (as shown in FIG. 5D). In embodiments, the first amplification product is further amplified to form a plurality of immobilized extension products. In embodiments, amplifying includes hybridizing the first immobilized polynucleotide to a second immobilized platform primer and extending the second platform primer to form a plurality of first amplification products (as shown in FIG. 4E). In embodiments, the second amplification product is further amplified to form a plurality of immobilized extension products. In embodiments, amplifying includes hybridizing the second immobilized polynucleotide to a third immobilized platform primer and extending the third platform primer to form a plurality of second amplification products (as depicted in FIG. 4F). In embodiments, amplifying includes a bridge amplification method (e.g., t-bPCR or c-bPCR). In embodiments, amplifying includes 1 to 100 bridge-PCR amplification cycles. In embodiments, amplifying includes a rolling circle amplification method (e.g., RCA or eRCA). In embodiments, amplifying includes 1 to 100 rolling circle amplification cycles.

In embodiments, the amplicons of a template polynucleotide originating from the population of third platform primers all include at least one cleavable site prior to contact with a cleaving agent (e.g. depicted in FIG. 4D). In embodiments, the cleavable site includes a diol linker, disulfide linker, photocleavable linker, abasic site, deoxyuracil triphosphate (dUTP), deoxy-8-oxo-guanine triphosphate (d-8-oxoG), methylated nucleotide, ribonucleotide, or a sequence containing a modified or unmodified nucleotide that is specifically recognized by a cleaving agent.

In embodiments, amplifying includes bridge polymerase chain reaction (bPCR) amplification, solid-phase rolling circle amplification (RCA), solid-phase exponential rolling circle amplification (eRCA), solid-phase recombinase polymerase amplification (RPA), solid-phase helicase dependent amplification (HDA), template walking amplification, or emulsion PCR on particles, or combinations thereof. In embodiments, amplifying includes a bridge polymerase chain reaction (bPCR) amplification. In embodiments, amplifying includes a thermal bridge polymerase chain reaction (t-bPCR) amplification. In embodiments, amplifying includes a chemical bridge polymerase chain reaction (c-bPCR) amplification. Chemical bridge polymerase chain reactions include fluidically cycling a denaturant (e.g., formamide) and maintaining the temperature within a narrow temperature range (e.g., +/−5° C.). In contrast, thermal bridge polymerase chain reactions include thermally cycling between high temperatures (e.g., 85° C.-95° C.) and low temperatures (e.g., 60° C.-70° C.). Thermal bridge polymerase chain reactions may also include a denaturant, typically at a much lower concentration than traditional chemical bridge polymerase chain reactions.

In embodiments, amplifying includes 1 to 100 bridge-PCR amplification cycles. In embodiments, amplifying includes 1 to 100 cycles of solid-phase rolling circle amplification (RCA), solid-phase exponential rolling circle amplification (eRCA), solid-phase recombinase polymerase amplification (RPA), solid-phase helicase dependent amplification (HDA), or template walking amplification. The method of claim 6, wherein amplifying includes 1 to 100 thermal bridge polymerase chain reaction (t-bPCR) amplification, chemical bridge polymerase chain reaction (c-bPCR) amplification or chemical-thermal bridge polymerase chain reaction (cT-bPCR) amplification).

In embodiments, the amplifying is at discrete locations in an ordered array of amplification sites on the surface. In some embodiments, the surface does not include an ordered array of amplification sites. For example, the surface may be uniformly coated with amplification primers, rather than coating some areas (amplification sites) and not others (interstitial regions).

In embodiments, the method further includes: (i) hybridizing and extending a first sequencing primer in a first sequencing cycle and detecting one or more labels in a first detection region to generate a sequencing read for the first template polynucleotide, wherein the first sequencing primer is complementary to the first sequencing primer binding sequence, and (ii) hybridizing and extending a second sequencing primer in a second sequencing cycle and detecting one or more labels in a second detection region to generate a sequencing read for the second template polynucleotide, wherein the second sequencing primer is complementary to the second sequencing primer binding sequence. In embodiments, the first and second detection regions are overlapping.

In embodiments, the method further includes (i) hybridizing and extending a first sequencing primer in a first sequencing cycle and detecting one or more labels in a first detection region to generate a sequencing read for the first template polynucleotide, wherein the first sequencing primer is complementary to the first sequencing primer binding sequence, and (ii) hybridizing and extending a second sequencing primer in a second sequencing cycle and detecting one or more labels in a second detection region to generate a sequencing read for the second template polynucleotide, wherein the second sequencing primer is complementary to the second sequencing primer binding sequence, and wherein the first and second detection regions are overlapping.

In some embodiments, methods provided herein include sequencing a template nucleic acid or amplicon described herein. The methods of template preparation and nucleic acid sequencing described herein can be incorporated into a suitable sequencing technique, non-limiting examples of which include SMRT (single-molecule real-time sequencing), ion semiconductor, pyrosequencing, sequencing by synthesis, combinatorial probe anchor synthesis, and SOLiD sequencing (sequencing by ligation). Non-limiting sequencing platforms include those provided by Singular Genomics™ (e.g., the G4™ sequencing platform), Illumina® (e.g., the MiniSeq™, MiSeq™, NextSeq™, and/or NovaSeq™ sequencing systems); Ion Torrent™ (e.g., the Ion PGM™, Ion S5™, and/or Ion Proton™ sequencing systems); Pacific Biosciences (e.g., the PACBIO RS II and/or Sequel II System sequencing system); ThermoFisher (e.g., a SOLiD® sequencing system); or BGI Genomics (e.g., DNBSeq™ sequencing systems). See, for example U.S. Pat. Nos. 7,211,390; 7,244,559; 7,264,929; 6,255,475; 6,013,445; 8,882,980; 6,664,079; and 9,416,409. In some embodiments, a sequencing method described herein does not include the use of SMRT sequencing or single-molecule sequencing.

In embodiments, the method includes sequencing the first and the second strand of a double-stranded template and/or amplification product by extending a sequencing primer hybridized thereto. A variety of sequencing methodologies can be used such as sequencing-by-synthesis (SBS), sequencing-by-binding, pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and. 6,274,320, each of which is incorporated herein by reference in its entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, that are present at features of an array are subjected to repeated cycles of oligonucleotide delivery and detection. SBL methods, include those described in Shendure et al. Science 309:1728-1732 (2005); U.S. Pat. Nos. 5,599,675; and 5,750,341, each of which is incorporated herein by reference in its entirety; and the SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which is incorporated herein by reference in its entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. A plurality of different nucleic acid fragments that have been attached at different locations of an array can be subjected to an SBS technique under conditions where events occurring for different templates can be distinguished due to their location in the array. In embodiments, the sequencing step includes annealing and extending a sequencing primer to incorporate a detectable label that indicates the identity of a nucleotide in the target polynucleotide, detecting the detectable label, and repeating the extending and detecting steps. In embodiments, the methods include sequencing one or more bases of a target nucleic acid by extending a sequencing primer hybridized to a target nucleic acid (e.g., an amplification product produced by the amplification methods described herein). In embodiments, the sequencing step may be accomplished by a sequencing-by-synthesis (SBS) process. In embodiments, sequencing includes a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. No. 10,738,072 and Chen et al, Proteomics & Bioinformatics, V. 11, Issue 1, 2013, Pages 34-40, each of which are incorporated herein by reference. Once such a modified nucleotide has been incorporated into the growing polynucleotide chain complementary to the region of the template being sequenced, there is no free 3′—OH group available to direct further sequence extension and therefore the polymerase cannot add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template. Non-limiting examples of suitable labels are described in U.S. Pat. Nos. 8,178,360, 5,188,934 (4,7-dichlorofluorscein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthene dyes): U.S. Pat. No. 5,688,648 (energy transfer dyes); and the like.

Sequencing includes, for example, detecting a sequence of signals. Examples of sequencing include, but are not limited to, sequencing by synthesis (SBS) processes in which reversibly terminated nucleotides carrying fluorescent dyes are incorporated into a growing strand, complementary to the target strand being sequenced. In embodiments, the nucleotides are labeled with up to four unique fluorescent dyes. In embodiments, the nucleotides are labeled with at least two unique fluorescent dyes. In embodiments, the readout is accomplished by epifluorescence imaging. A variety of sequencing chemistries are available, non-limiting examples of which are described herein.

In embodiments, sequencing is performed according to a “sequencing-by-binding” method (see, e.g., U.S. Pat. Pubs. US2017/0022553 and US2019/0048404, each of which is incorporated herein by reference in its entirety), which refers to a sequencing technique wherein specific binding of a polymerase and cognate nucleotide to a primed template nucleic acid molecule (e.g., blocked primed template nucleic acid molecule) is used for identifying the next correct nucleotide to be incorporated into the primer strand of the primed template nucleic acid molecule. The specific binding interaction need not result in chemical incorporation of the nucleotide into the primer. In some embodiments, the specific binding interaction can precede chemical incorporation of the nucleotide into the primer strand or can precede chemical incorporation of an analogous, next correct nucleotide into the primer. Thus, detection of the next correct nucleotide can take place without incorporation of the next correct nucleotide. As used herein, the “next correct nucleotide” (sometimes referred to as the “cognate” nucleotide) is the nucleotide having a base complementary to the base of the next template nucleotide. The next correct nucleotide will hybridize at the 3′-end of a primer to complement the next template nucleotide. The next correct nucleotide can be, but need not necessarily be, capable of being incorporated at the 3′ end of the primer. For example, the next correct nucleotide can be a member of a ternary complex that will complete an incorporation reaction or, alternatively, the next correct nucleotide can be a member of a stabilized ternary complex that does not catalyze an incorporation reaction. A nucleotide having a base that is not complementary to the next template base is referred to as an “incorrect” (or “non-cognate”) nucleotide.

In embodiments, sequencing includes a plurality of sequencing cycles. In embodiments, sequencing includes 10 to 100 sequencing cycles. In embodiments, sequencing includes 50 to 100 sequencing cycles. In embodiments, sequencing includes 50 to 300 sequencing cycles. In embodiments, sequencing includes 50 to 150 sequencing cycles. In embodiments, sequencing includes at least 10, 20, 30 40, or 50 sequencing cycles. In embodiments, sequencing includes at least 10 sequencing cycles. In embodiments, sequencing includes 10 to 20 sequencing cycles. In embodiments, sequencing includes 10, 11, 12, 13, 14, or sequencing cycles. In embodiments, sequencing includes (a) extending a sequencing primer by incorporating a labeled nucleotide, or labeled nucleotide analogue and (b) detecting the label to generate a signal for each incorporated nucleotide or nucleotide analogue. In embodiments, detecting includes two-dimensional (2D) or three-dimensional (3D) fluorescent microscopy. Suitable imaging technologies are known in the art, as exemplified by Larsson et al., Nat. Methods (2010) 7:395-397 and associated supplemental materials, the entire content of which is incorporated by reference herein in its entirety. In embodiments of the methods provided herein, the imaging is accomplished by confocal microscopy. Confocal fluorescence microscopy involves scanning a focused laser beam across the sample, and imaging the emission from the focal point through an appropriately-sized pinhole. This suppresses the unwanted fluorescence from sections at other depths in the sample. In embodiments, the imaging is accomplished by multi-photon microscopy (e.g., two-photon excited fluorescence or two-photon-pumped microscopy). Unlike conventional single-photon emission, multi-photon microscopy can utilize much longer excitation wavelength up to the red or near-infrared spectral region. This lower energy excitation requirement enables the implementation of semiconductor diode lasers as pump sources to significantly enhance the photostability of materials. Scanning a single focal point across the field of view is likely to be too slow for many sequencing applications. To speed up the image acquisition, an array of multiple focal points can be used. The emission from each of these focal points can be imaged onto a detector, and the time information from the scanning mirrors can be translated into image coordinates. Alternatively, the multiple focal points can be used just for the purpose of confining the fluorescence to a narrow axial section, and the emission can be imaged onto an imaging detector, such as a CCD, EMCCD, or s-CMOS detector. A scientific grade CMOS detector offers an optimal combination of sensitivity, readout speed, and low cost. One configuration used for confocal microscopy is spinning disk confocal microscopy. In 2-photon microscopy, the technique of using multiple focal points simultaneously to parallelize the readout has been called Multifocal Two-Photon Microscopy (MTPM). Several techniques for MTPM are available, with applications typically involving imaging in biological tissue. In embodiments of the methods provided herein, the imaging is accomplished by light sheet fluorescence microscopy (LSFM). In embodiments, detecting includes 3D structured illumination (3DSIM). In 3DSIM, patterned light is used for excitation, and fringes in the Moird pattern generated by interference of the illumination pattern and the sample, are used to reconstruct the source of light in three dimensions. In order to illuminate the entire field, multiple spatial patterns are used to excite the same physical area, which are then digitally processed to reconstruct the final image. See York, Andrew G., et al. “Instant super-resolution imaging in live cells and embryos via analog image processing.” Nature methods 10.11 (2013): 1122-1126 which is incorporated herein by reference. In embodiments, detecting includes selective planar illumination microscopy, light sheet microscopy, emission manipulation, pinhole confocal microscopy, aperture correlation confocal microscopy, volumetric reconstruction from slices, deconvolution microscopy, or aberration-corrected multifocus microscopy. In embodiments, detecting includes digital holographic microscopy (see for example Manoharan, V. N. Frontiers of Engineering: Reports on Leading-edge Engineering from the 2009 Symposium, 2010, 5-12, which is incorporated herein by reference). In embodiments, detecting includes confocal microscopy, light sheet microscopy, or multi-photon microscopy.

### EXAMPLES

### Example 1. Monoclonal Clustering

Next generation sequencing (NGS) methods often rely on the amplification of genomic fragments hybridized to polynucleotide primers on a solid surface, referred to as amplification sites. Ideally these amplification sites have one initial template fragment at a given feature (e.g., site on a flowcell, such as within a well, on a particle, or both on a particle in a well) that is then amplified to occupy the entire feature. However, instances of polyclonal sites, (i.e., where more than one distinct polynucleotide is present and amplified) negatively impact sequencing results by increasing sequencing duplications or simultaneous interfering signaling.

Hybridizing a target polynucleotide to a polynucleotide primer is an inherently stochastic event. For stochastic events occurring over a period of time (e.g., a seeding-amplification cycle) it may be convenient to use the Poisson approximation to better understand the probability of an event occurring during that time. For example, if one knows the average rate of a hybridizing event, represented as λ^{seed}, (i.e., how often a target polynucleotide hybridizes to a polynucleotide primer) occurring during a seeding-amplification cycle, it is possible to calculate the probability that an amplification site will contain an amplicon (e.g., a monoclonal amplicon) following a seeding-amplification cycle. Two variables affecting λ^{seed} include the concentration target polynucleotide and the amount of time the target polynucleotide is exposed to the polynucleotide primer, t_{seed}, during a seeding-amplification cycle. Generally, increasing the concentration of the target polynucleotide or increasing t_{seed} increases λ^{seed}.

Conventional methods typically overseed an array of available sites, that is, the methods typically used ensure the concentration of the target polynucleotides are in abundance relative to the available amplification sites to maximize the opportunity for a target polynucleotide to hybridize to the primer in the amplification site. Unfortunately, this results in polyclonal amplicons (i.e., two or more populations of distinct fragment amplicons) forming in the amplification site. Polyclonal amplicons result in poor quality sequencing due to the fact that multiple templates are present, in contrast to monoclonal clusters, which have only one template per spot (i.e., one template per feature). Increasing the proportion of monoclonal clusters on a solid support, such as a flow cell, for example, will increase the total read output of a sequencing run, increase the confidence of a correctly called base therefore increasing the quality score (i.e., accuracy), and reduce the cost per sequencing read.

Existing methods to overcome polyclonality have been described, and include kinetic exclusion amplification (see, e.g., U.S. Pat. Pubs. US2017/0335380 and US2018/0037950, each of which are incorporated herein by reference), which involves the use of an amplification reaction wherein the seeding process proceeds at a slower rate than the clustering process. Seeded spots are fully clustered before they might be reseeded by a different template. Kinetic exclusion amplification requires that the number of target nucleic acids in the seeding solution be greater than the number of spots that may be seeded. An alternative method, referred to herein as staircase amplification (see, e.g., U.S. Pat. Pub. US2018/0044732, which is incorporated herein by reference, relies on repeated rounds of template seeding and clustering of a subset of flow cell spots to increase the seeding density and reduce polyclonality.

Embodiments of the invention described herein make significant advances over existing clustering methods (e.g., staircase amplification and kinetic exclusion amplification) and produce a higher fraction of monoclonal clusters. The methods described herein are referred to as “delayed onset amplification”, and include seeding a plurality of template polynucleotides onto a plurality of immobilized surface primers, wherein at least one of the surface primers includes a cleavable site (e.g., a uracil) at the 3′ end. Following two rounds of extension, the cleavable site is cleaved (e.g., cleavage of a uracil by uracil DNA glycosylase treatment and heat cycling under alkaline conditions), wherein the 3′ end of the cleaved primer is blocked for further extension. Additional rounds of amplification (e.g., chemical bridge PCR) are performed, wherein only the unblocked surface primers are extended. Subsequently, the 3′ end of the blocked surface primers are unblocked and used in subsequent amplification, but only in wells that did not give rise to clusters during the initial amplification step (i.e., wells seeded with two species of templates would only have one species amplified prior to primer unblocking, and subsequently would not support additional amplification). This process leads to increased proportions of monoclonal amplicons on a solid support (e.g., a flow cell), even in wells seeded with a plurality of different templates.

### Example 2. Delayed Onset Amplification

As described supra, amplification sites on a solid support ideally have one copy (i.e., are monoclonal) of a hybridized polynucleotide fragment, however instances of polyclonal sites, (i.e., where more than one distinct polynucleotide is present) are common and interfere with sequencing results. Increasing the proportion of monoclonal clusters on a flow cell, for example, will increase the total quality and read output of a sequencing run, and reduce the cost per read.

Existing methods to overcome polyclonality include kinetic exclusion amplification which involves the use of an amplification reaction wherein the seeding process proceeds at a slower rate than the clustering process. Seeded spots are fully clustered before they might be reseeded, reducing polyclonality. Kinetic exclusion amplification requires that the number of target nucleic acids in the seeding solution be greater than the number of spots that may be seeded. An alternative method, referred to herein as staircase amplification, relies on repeated rounds of template seeding and clustering of a subset of flow cell spots to increase the seeding density and reduce polyclonality, but is dependent on the library concentration seeded.

Prior to ligation, adenylation of fragmented and end-repaired nucleic acids (e.g., genomic DNA that has been fragmented and end-repaired) using a polymerase, which lacks 3′-5′ exonuclease activity, is often performed in order to minimize chimera formation and adapter-adapter (dimer) ligation products. In these methods, single 3′ A-overhang DNA fragments are ligated to single 5′ T-overhang adapters, whereas A-overhang fragments and T-overhang adapters have incompatible cohesive ends for self-ligation. During size selection, fragments of undesired size are eliminated from the library using gel or bead-based selection in order to optimize the library insert size for the desired sequencing read length. This often maximizes sequence data output by minimizing overlap of paired end sequencing that occurs from short DNA library inserts. Amplifying libraries prior to NGS analysis is typically a beneficial step to ensure there is a sufficient quantity of material to be sequenced.

Embodiments of the adapter oligonucleotide sequences contemplated herein include those shown in FIG. 1, referred to as P1, P2, and P3 adapters, respectively. The illustrations depict embodiments of the oligonucleotide sequences, wherein there are three different platform primer sequences, pp1, pp2, and pp3, in combination with three different sequencing primer binding sites: SP1, SP2, and SP3. Any P1 adapter, or the complement thereof, may be combined with any P2 or P3 adapter, or complement thereof, when preparing the template nucleic acid sequence. The 5′ end of any of the adapters shown in FIG. 1 may be covalently attached to a solid surface via a linker (not shown).

In some aspects of a method herein, an adapter-target-adapter nucleic acid template (FIGS. 2A-2B) is provided where two adapters are ligated to each respective end of a polynucleotide duplex. A polynucleotide duplex refers to a double-stranded portion of a polynucleotide, for example, a cDNA polynucleotide desired to be sequenced. Each adapter is a Y adapter (alternatively, this may be referred to as a mismatched adapter or a forked adapter) that is ligated to one end of a polynucleotide duplex. The adapter is formed by annealing two single-stranded oligonucleotides, such as P1 and P2′. FIG. 2A shows a DNA template with P1 and P2′ adapters ligated to the ends when hybridized together (top), and the subsequent amplification products (bottom). P1 and P2′ may be prepared by a suitable automated oligonucleotide synthesis technique. The oligonucleotides are partially complementary such that a 3′ end and/or a 3′ portion of P1 is complementary to the 5′ end and/or a 5′ portion of P2′. A 5′ end and/or a 5′ portion of P1 and a 3′ end and/or a 3′ portion of P2′ are not complementary to each other, in certain embodiments. When the two strands are annealed, the resulting Y adapter is double-stranded at one end (the double-stranded region) and single-stranded at the other end (the unmatched region), and resembles a ‘Y’ shape. FIG. 2B shows a DNA template with P1 and P3′ adapters ligated to the ends when hybridized together (top) and the subsequent amplification products (bottom). As illustrated, two Y-shaped adapters are ligated to the sample polynucleotide, however it is understood that alternative shaped adapters are contemplated herein (e.g., hairpin adapters, blunt end adapters, bubble adapters, and the like). In embodiments, each end of the sample polynucleotide is ligated to adapters having the same shape (e.g., both ends include a Y-adapter). In embodiments, each end of the sample polynucleotide is ligated to adapters having different shapes (e.g., the first adapter is a Y adapter and the second adapter is a hairpin adapter).

The single-stranded portions (the unmatched regions) of both P1 and P2′ have an elevated melting temperature (Tm) (e.g., about 75° C.) relative to their respective complements to enable efficient binding of surface primers and stable binding of sequencing primers. In contrast to the single-stranded portions, a double-stranded region, in certain embodiments, has a moderate Tm (e.g., 40-45° C.) so that it is stable during ligation. In embodiments, a double-stranded region has an elevated Tm (e.g., 60-70° C.). In embodiments, the GC content of the double-stranded region is >50% (e.g., approximately 60-75% GC content). The unmatched region of P1 and P2′, in certain embodiments, are about 25-35 nucleotides (e.g., nucleotides), whereas the double-stranded region is shorter, ranging about 10-20 nucleotides (e.g., 13 nucleotides) in total. In embodiments, the unmatched region of P1 and P2′ are about 35-60 nucleotides (e.g. 60 nucleotides).

A ligation reaction between the Y adapters and the cDNA fragments is then performed using a suitable ligase enzyme (e.g., T4 DNA ligase), which joins two Y adapters to each DNA fragment, one at either end, to form adapter-target-adapter constructs. A mixture of adapter sequences are utilized (as depicted in FIG. 1) during the target-adapter ligation step, such that a defined number of unique adapters are present. The products of this reaction can be purified from leftover unligated adapters by a number of means (e.g., NucleoMag NGS Clean-up and Size Select kit, Solid Phase Reversible Immobilization (SPRI) bead methods such as AMPureXP beads, PCRclean-dx kit, Axygen AxyPrep FragmentSelect-I Kit), including size-inclusion chromatography, preferably by electrophoresis through an agarose gel slab followed by excision of a portion of the agarose that contains the DNA greater in size that the size of the adapter.

Once formed, the library of adapter-target-adapter templates prepared according to the methods described above can be used for solid-phase nucleic acid amplification. Illustrated in FIG. 3 is a pattered solid support containing a plurality of features. Each feature includes a plurality of immobilized oligonucleotides, referred to as platform primer oligonucleotides. Within each feature, as depicted in FIG. 3, the plurality of immobilized oligonucleotides includes a first platform primer oligonucleotide (pp1) having complementarity to all or a portion of P1, a second platform primer oligonucleotide (pp2) having complementarity to all or a portion of P2, and a third platform primer oligonucleotide (pp3) having complementarity to all or a portion of P3. In embodiments, each feature includes a plurality of immobilized oligonucleotides. In embodiments, the plurality includes include a first population of platform primer oligonucleotides (pp1) having complementarity to all or a portion of P1, or the complement thereof; a second population of platform primer oligonucleotides (pp2) having complementarity to all or a portion of P2, or the complement thereof; and a third population of platform primer oligonucleotides (pp3) having complementarity to all or a portion of P3, or the complement thereof. The third platform primer oligonucleotides includes one or more cleavable sites, depicted as the plaque shape in FIG. 3.

The prepared library molecules are allowed to contact the solid support and 0, 1, 2, or more molecules may contact a single feature. For example, if one molecule seeds (i.e., hybridize to the surface-immobilized oligonucleotide) a single feature and is amplified it is referred to as a monoclonal colony. Monoclonal colony formation for a P1′-template-P2 molecule is illustrated in FIGS. 4A-4B and 4E, where an initial molecule anneals to a first surface-immobilized oligonucleotide and is extended to form an immobilized extension product. The initial molecule is removed and the immobilized extension product hybridizes to a second surface-immobilized oligonucleotide, and with a polymerase is extended to form a second immobilized extension product (FIG. 4B). Under suitable amplification conditions, the process is repeated to form a plurality of immobilized extension product, as illustrated in FIG. 4E. A similar process occurs for P1-template-P3′ molecules (FIG. 4C-4D) to generate a monoclonal colony in a feature, of which the final product is exemplified in FIG. 4F.

Reducing polyclonality in a feature may be accomplished, for example, as illustrated in FIG. 5A, which shows seeding and extension of two molecules, a P1′-template-P2 molecule (left) and a P1′-template-P3 molecule (right). In embodiments, the third platform primer oligonucleotides (i.e., pp3) includes one or more cleavable sites, depicted as the plaque shape. An additional round of extension, whereby the immobilized extension products anneal and to another surface-immobilized oligonucleotide (FIG. 5B), and with a polymerase is extended to form additional immobilized extension products (FIG. 5C). The cleavable site on the platform primer oligonucleotides does not preclude hybridization or extension. The surface-immobilized oligonucleotides and extension products including a cleavable site are cleaved and additional rounds of amplification (FIG. 5D) are performed to enable the P1-template-P2′ containing amplification products to dominate the feature. Cleaving the cleavable site prevents extension of the cleaved primers by a polymerase, but hybridization is still permitted.

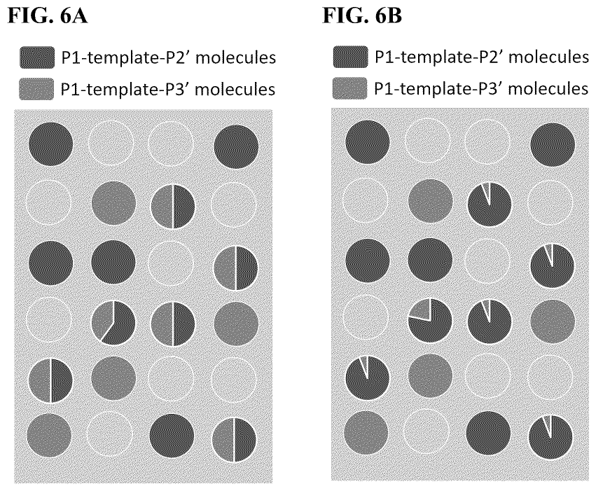

In some embodiments, every well on a multiwell plate contains equal proportions of three surface primers. For example, the following three surface primers are immobilized in each well of a multiwell plate in equal proportions: a P1 primer, a P2 primer, and a P3 primer, wherein the P3 primer has a uracil at a 3′ end. Template nucleic acids are then seeded (e.g., template nucleic acids containing adapter sequences complementary to the immobilized surface primers are hybridized to the surface) at approximately a 90% occupancy. Shown in FIG. 6A, for example, is a 4×6 patterned array (e.g., a multiwell plate) following an initial seeding event (i.e., wherein a plurality of library molecules contact the solid support). The outcome of seeding at an equal ratio of molecules to available sites, referred to as 1:1 seeding, estimates about 37% of the available sites will be empty (empty circles), about 37% of the available sites are contacted by a single molecule (solid color circles), about 18% hybridize two molecules (represented as a circle containing two different colors with equal portion), and about 8% contain three or more different molecules (represented as a circle containing two different colors with unequal portion). Following template seeding, a first extension of all seeded templates is performed, generating immobilized complements of each seeded template. A second extension is then performed to generate an immobilized template nucleic acid. As described supra, this is then followed by UDG treatment to excise the uracil from the P3 primer and a short heat-treatment step to cleave the abasic site, leaving behind P3 primers blocked for extension by a 3′-phosphate (i.e., any P3-containing amplicon would be prevented from amplification following UDG/heat-treatment).

Solid phase amplification is then performed, for example 40 cycles or less of c-bPCR, generating a plurality of P1- and P2-containing amplification products in wells containing amplicons with P1 and P2′ adapter sequences. At this stage, none of the amplicons containing P3 adapter sequences (e.g., templates with a P1 adapter and a P3 adapter on the ends) have been amplified, as the cleaved P3 primers are still blocked and unable to be extended. Free P1 surface primers would be consumed during the c-bPCR process in wells with P1-containing immobilized templates (e.g., templates with a P1 adapter and a P2′ adapter on the ends). Amplification of the P1-containing templates, in wells seeded with both template species (e.g., diclonal-seeded wells with P1-P2′ and P1-P3′ template molecules), will subsequently prevent amplification of P1-P3′ template molecules due to the lack of available P1 surface primer. The 3′-end of the cleaved P3 surface primers are then dephosphorylated with, for example, T4 Polynucleotide Kinase (PNK), and amplification of P3-containing molecules is performed, but only in wells containing P3 and P1 surface primers left for efficient amplification (i.e., wells that did not use up free P1 surface primer for amplifying templates containing P1 and P2 adapter sequences). FIG. 6B illustrates the reduction in polyclonality of the seeded array (e.g., an array seeded with P1-template-P2′ and P1-template-P3′ molecules, as shown in FIG. 6A) following the method described herein. Wells that were initially di-clonal (containing both template species) would shift to being predominantly monoclonal due to the amplification cycles performed wherein the P3 primer was blocked, leading to enrichment of the P1 and P2-containing molecules.

These approaches will aid in converting polyclonal clusters into a greater proportion of monoclonal clusters. Reducing the distribution and frequency of polyclonal amplicons while increasing the density and proportion of monoclonal spots will result in significant improvements in sequencing throughput, accuracy, and reduced cost. In addition to increasing the throughput of sequencing chips, the method may be used as part of a chip production step to convert a conventional flow cell into a flow cell containing spots having one of a predetermined number of target specific oligonucleotide sequences. This would enable applications such as SNP sequencing for genotyping, large gene expression panels, and facilitate the production of customized targeted sequencing panels. The method described herein could also be used as part of the creation of DNA hybridization-based microarrays.

What is claimed is:

1. A method of amplifying a polynucleotide on a solid support comprising a plurality of immobilized primers, said method comprising:
hybridizing a second platform primer binding sequence of a first immobilized polynucleotide to a second immobilized primer; wherein the first immobilized polynucleotide comprises a first platform primer sequence immobilized to a solid support, a template sequence, and said second platform primer binding sequence;
hybridizing a third platform primer binding sequence of a second immobilized polynucleotide to a third immobilized primer comprising a cleavable site; wherein the second immobilized polynucleotide comprises the first platform primer sequence, a template sequence, and said third platform primer binding sequence;
extending the second immobilized primer with a polymerase to form a first amplification product and extending the third immobilized primer with a polymerase to form a second amplification product comprising the cleavable site;
cleaving the cleavable site and removing the second amplification product; and
amplifying the first amplification product and the first immobilized polynucleotide.

2. A method of forming a first immobilized polynucleotide and a second immobilized polynucleotide on a solid support, said method comprising:
contacting a solid support with a first polynucleotide and a second polynucleotide, wherein the solid support comprises a population of first platform primers, a population of second platform primers, and a population of third platform primers, wherein each third platform primer comprises a cleavable site and wherein each of said first platform primers, said second platform primers and said third platform primers are immobilized to said solid support;
hybridizing a first platform primer binding sequence of the first polynucleotide to one of said first platform primers, wherein the first polynucleotide comprises said first platform primer binding sequence, a template sequence, and a second platform primer sequence;
hybridizing a first platform primer binding sequence of the second polynucleotide to one of said first platform primers, wherein the second polynucleotide comprises said first platform primer binding sequence, a template sequence, and a third platform primer sequence;
extending the first platform primer with a polymerase to form the first immobilized polynucleotide comprising the first platform primer sequence, a complement of the template sequence, and a second platform primer binding sequence;
extending the second platform primer with a polymerase to form the second immobilized polynucleotide comprising the first platform primer sequence, a complement of the template sequence, and a third platform primer binding sequence.

3. The method of claim 2, further comprising hybridizing the first immobilized polynucleotide to one of said second platform primers and extending the second platform primer to form a first amplification product, and hybridizing the second immobilized polynucleotide to one of said third platform primers and extending the third platform primer to form a second amplification product comprising the cleavable site.

4. The method of claim 3, further comprising cleaving the cleavable site and removing the second amplification product from said solid support.

5. The method of claim 3, further comprising amplifying the first amplification product and the second amplification product.

6. The method of claim 1, wherein amplifying comprises 1 to 100 bridge-PCR amplification cycles.

7. The method of claim 1, wherein amplifying results in higher ratio of first immobilized polynucleotide and first amplification product relative to the second immobilized polynucleotide and second amplification product.

8. The method of claim 1, wherein the cleavable site comprises a diol linker, disulfide linker, photocleavable linker, abasic site, deoxyuracil triphosphate (dUTP), deoxy-8-Oxo-guanine triphosphate (d-8-oxoG), methylated nucleotide, ribonucleotide, or a sequence containing a modified or unmodified nucleotide that is specifically recognized by a cleaving agent.

9. The method of claim 1, wherein cleaving the cleavable site comprises contacting said cleavable site with a cleaving agent.

10. The method of claim 9, wherein the cleaving agent is sodium periodate, RNase, formamidopyrimidine DNA glycosylase (Fpg), endonuclease, uracil DNA glycosylase (UDG), TCEP, THPP, sodium dithionite (Na₂S₂O₄), hydrazine (N₂H₄), Pd(0), or ultraviolet radiation.

11. The method of claim 1, wherein amplifying comprises 1 to 100 cycles of solid-phase rolling circle amplification (RCA), solid-phase exponential rolling circle amplification (eRCA), solid-phase recombinase polymerase amplification (RPA), solid-phase helicase dependent amplification (HDA), or template walking amplification.

12. The method of claim 6, wherein amplifying comprises 1 to 100 thermal bridge polymerase chain reaction (t-bPCR) amplification, chemical bridge polymerase chain reaction (c-bPCR) amplification or chemical-thermal bridge polymerase chain reaction (cT-bPCR) amplification).

13. A solid support comprising a plurality of amplification sites, wherein each amplification site comprises a population of first platform primers, a population of second platform primers, and a population of third platform primers, wherein
each of the third platform primers comprise a cleavable site,
each of said populations have a different platform primer binding sequence relative to each population, and
each of said different populations have a common platform primer binding sequence within each population.

14. The solid support of claim 13 wherein the solid support is selected from a flow cell, bead, chip, capillary, plate, membrane, wafer, comb, pin, nanoparticle, multi-well container, or unpatterned solid support.

15. The solid support of claim 13 wherein the solid support further comprises a polymer, photoresist or hydrogel layer.

16. The solid support of any of claim 13, wherein the solid support further comprises a plurality of immobilized oligonucleotides.

17. A kit comprising the solid support of claim 13.

18. The kit of claim 17, further comprising a first oligonucleotide comprising a first platform primer binding sequence, a second oligonucleotide comprising a second platform primer binding sequence, and a third oligonucleotide comprising a third platform primer binding sequence.

19. The kit of claim 17, further comprising a polymerase and a plurality of deoxynucleotides (dNTPs).

20. The kit of claim 18, wherein the first oligonucleotide further comprises a first sequencing primer binding sequence and optionally an index sequence, the second oligonucleotide further comprises a second sequencing primer binding sequence and optionally an index sequence, and third oligonucleotide further comprises a second sequencing primer binding sequence and an optionally an index sequence.
